(19) United States
(12) Patent Application Publication
(10) Pub. No.: US 2026/0250256 A1
(43) Pub. Date: Aug. 27, 2026

(54) COMPOUNDS FOR POSITIVE MODULATION OF THE AUTOPHAGY-LYSOSOMAL PATHWAY AND METHODS OF USE

(71) Applicant: The University of North Carolina at Pembroke, Pembroke, NC (US)

(72) Inventors: Ben A. Bahr, Southport, NC (US); Philippe Chemla, Durham, NC (US); Jason Alexander King, Wilmington, NC (US); Rajababu Kanuganti, Wilmington, NC (US); Rajendra Kumar Reddy Gadikota, Wilmington, NC (US)

(21) Appl. No.: 19/648,933

(22) Filed: Apr. 15, 2026

Related U.S. Application Data
(63) Continuation-in-part of application No. PCT/US2025/050375, filed on Oct. 9, 2025.
(60) Provisional application No. 63/705,396, filed on Oct. 9, 2024.

(51) Int. Cl. C07D 265/06; A61K 31/40; A61K 31/4015; A61K 31/4025; A61K 31/405; A61K 31/426; A61K 31/428; A61K 31/4406; A61K 31/453; A61K 31/535; A61K 31/5355; A61K 31/5377; A61P 25/28; C07D 207/08; C07D 207/46; C07D 209/08; C07D 209/20; C07D 213/38; C07D 277/28; C07D 405/06; C07D 413/06; C07D 417/12

(52) U.S. Cl. CPC C07D 265/06; A61K 31/40; A61K 31/4015; A61K 31/4025; A61K 31/405; A61K 31/426; A61K 31/428; A61K 31/4406; A61K 31/453; A61K 31/535; A61K 31/5355; A61K 31/5377; A61P 25/28; C07D 207/08; C07D 207/46; C07D 209/08; C07D 209/20; C07D 213/38; C07D 277/28; C07D 405/06; C07D 413/06; C07D 417/12

(57) ABSTRACT

Disclosed are compounds of Formulas (I), (Ia), (Ib), (II), (IIa), (III), (IIIa), and (IIIb), as well as pharmaceutical compositions thereof. The compounds can be used to improve proteostasis and enhance clearance of protein accumulation events by positively modulating the autophagy-lysosomal pathway, including augmenting the activity of cathepsin enzymes, and/or to treat neurological diseases, disorders and conditions, such as, but not limited to, Alzheimer's disease, Parkinson's disease, Huntington's disease, mild cognitive impairment, frontotemporal dementia, amyotrophic lateral sclerosis, Lewy body dementias, chronic traumatic encephalopathy, traumatic brain injury, and α-synucleinopathies.

### CROSS REFERENCE TO RELATED APPLICATIONS

This application is a continuation-in-part of PCT International Patent Application No. PCT/US2025/050375, filed Oct. 9, 2025, which claims the priority benefit of U.S. Provisional Patent Application Ser. No. 63/705,396, filed Oct. 9, 2024, the disclosure of each if which is incorporated herein by reference in its entirety.

### ABBREVIATIONS

%=percent
α-syn=α-synuclein
μM=micromolar
Aβ=amyloid beta
Aβ42=42 amino acid form of Aβ
AD=Alzheimer's disease
Ala=alanine
ALS=amyotrophic lateral sclerosis
Arg=arginine
APP=amyloid beta precursor protein
CA=Cornu Ammonis
CatB=cathepsin B
CQN=chloroquine
CTF=carboxyterminal fragment of APP
DG=dentate gyrus
DLB=dementia with Lewy bodies
EC₅₀=50% inhibitory concentration
FTD=frontotemporal dementia
HD=Huntington's disease
kDa=kilodalton
kg=kilogram
MCI=mild cognitive impairment
mg=milligram
mm=millimeter
MW=molecular weight
nt=not treated
PADK=Z-Phe-Ala-diazomethylketone
PD=Parkinson's disease
PDD=Parkinson's disease dementia
PET=positron emission tomography
Phe=phenylalanine
pTau=phosphorylated tau
sg=stratum granulosum
sp=stratum pyramidale.
TBI=traumatic brain injury
Z (or CBZ)=benzyloxycarbonyl

### BACKGROUND

The growing trend toward aging populations throughout the world has resulted in a dramatic rise in the occurrence of i) Alzheimer's disease (AD; represents 60-70% of all dementia cases), ii) other age-related dementias, and iii) the associated socio-economic stresses in families, cities, and countries (now affecting one in nine older adults in the U.S.). Alzheimer's disease (AD) is characterized by progressive loss of cognitive function. AD is one of a class of disorders referred to as protein accumulation disorders, which are believed to involve imbalances between protein production and protein clearance. In addition to AD, these disorders include frontotemporal dementia (FTD), Parkinson's disease (PD), Huntington's disease (HD), amyotrophic lateral sclerosis (ALS), and other dementias.

Intravenous immunotherapies that target Aβ42-related pathology have recently been approved by the FDA for treating AD. However, deposits of both the Aβ42 peptide and pathogenic tau protein aggregates occur in 100% of AD cases. In addition, more than 50% of AD cases exhibit α-synuclein and other protein accumulations. Thus, AD is a distinct multi-proteinopathy, likely involving several proteins and their corrupted deposition. In addition to AD, tau pathology is also found in several other common neurodegenerative diseases, including dementias referred to as “tauopathies” (such as FTD), while abnormal α-synuclein accumulation causes α-synucleinopathies, which include Lewy body dementia, an umbrella term for two related clinical diagnoses, “dementia with Lewy bodies (DLB)” and “Parkinson's disease dementia (PDD),” the latter having more severe cognitive challenges than those in PD.

With many advances occurring for the early detection of AD-related pathology that can occur 10-15 years before cognitive symptoms (e.g., PET scans for Aβ42 and tau), as well as for early PD detection (e.g., smell loss test, which precedes PD diagnosis by up to 10 years), there is an ongoing need for therapeutic agents (e.g., orally available therapeutic agents) to treat AD, FTD, PD, PDD, and DLB. There is also an ongoing need for therapeutic agents to treat the many individuals at risk of developing dementia among the growing number of elderly (such as those living with mild cognitive impairment (MCI)), e.g., to lower their dementia risk, slow dementia onset and progression, and promote healthy cognitive aging.

### SUMMARY

In some examples, the presently disclosed subject matter provides compounds of Formulas (I), (Ia), (Ib), (II), (IIa), (III), (IIIa), and (IIIb) as described hereinbelow, as well as pharmaceutical compositions thereof. In some examples, the compounds can comprise a substituted 1,3-oxazin-4-one moiety or an analog thereof. In some examples, the compounds can comprise a substituted benzopyrone moiety or an analog thereof. In some examples, the compound is selected from the group consisting of SR466, SR71, SR70, SR72, SR401, SR459, SR466, SR462, SR103, SR103B, SR104, SR105, SR107, SR111, SR720, SR720-1, SRB3, SR70, SR71, SR72, SR4721, SR4722, SR7254, SR7280, SR7255, SR5955, SR5981, SR-J1, SR438, SR498, SR490, SR495, SR496, SR206, SR4386, SR4385, SR4384, SR4511, SR7301, SR7302, SR7232, and SR710, optionally SR459, SR466, SR462, and SR438. In some examples, the compound is SR438, SR498, SR459 or SR466, optionally wherein the compound is SR438-S, SR438-R, SR498-S, SR498-R, SR459-S, SR459-R, SR466-S, or SR466-R. In some examples, the compound is SR4722, isomer 1 and/or SR4722, isomer 2. In some examples, the compounds can be used for to improve proteostasis and enhance clearance of protein accumulation events by positively modulating the autophagy-lysosomal pathway, including augmenting the activity of cathepsin enzymes, to treat neurological diseases, disorders and conditions, such as, but not limited to, Alzheimer's disease, Parkinson's disease, Huntington's disease, mild cognitive impairment, frontotemporal dementia, amyotrophic lateral sclerosis, Lewy body dementias, chronic traumatic encephalopathy, traumatic brain injury, and α-synucleinopathies.

Accordingly, it is an object of the presently disclosed subject matter to provide compounds and pharmaceutical compositions for use in positively modulating cathepsin B, enhancing the autophagy-lysosomal pathway for proteostasis, and/or treating neurological diseases, disorders, and conditions.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A and 1B: Rat hippocampal explants for screening compounds. The organotypic hippocampal transverse slice cultures were maintained on Biopore inserts for 3-6 weeks, exhibiting native organization of Nissl-stained neuronal subfields. FIG. 1A shows an image with a view-field width of 1.5 mm. Abbreviations are as follows: CA, Cornu Ammonis; DG, dentate gyrus; sg, stratum granulosum; sp, stratum pyramidale. FIG. 1B shows exemplary anti-CatB immunoblots for samples from matured explants treated with or without 10 μM PADK, SR-440, SR-459, or 10-30 M SR-438 for 48 hours. Slice cultures were harvested into samples of 6-8 slices each, homogenized, and equal protein aliquots assessed by immunoblot for cathepsin B (CatB) isoforms: the 30-kDa isoform (CatB-30) and the 25-kDa isoform (CatB-25).

FIG. 2: Dose-dependent increase in cathepsin B activity by SR-459. Organotypic hippocampal cultures were treated with different concentrations of SR-459 for 48 h. Prepared tissue samples from the harvested slice cultures were subjected to the CalBiochem cathepsin B assay kit that utilizes the fluorogenic substrate Z-Arg-Arg AMC. Mean fluorescence units±SEM were normalized to percent of vehicle-treated control sample activity, and the data were plotted and evaluated by GraphPad Prism to determine the EC₅₀ (range of 0.79 to 2.08 μM, R=0.913, p<0.05).

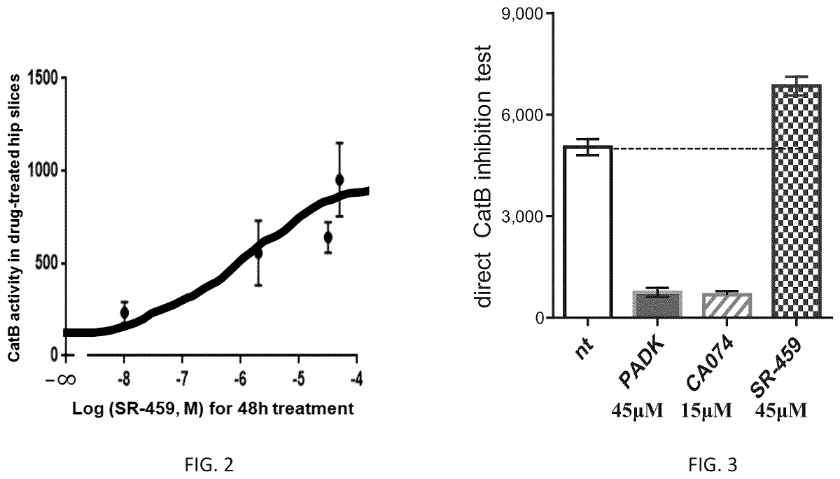

FIG. 3: Different classes of CatB-targeting compounds were tested for direct action on CatB activity in lysed cells. Equal amounts of lysed material were either not treated (nt) or incubated with PADK (a weak CatB inhibitor), potent CatB inhibitor CA074, or SR-459. The cathepsin B assay with fluorogenic substrate Z-Arg-Arg AMC determined the plotted mean fluorescence units±SEM which were evaluated by GraphPad Prism. Compared to the nt control, 45 M SR-459 increased CatB activity by 49.9% (p=0.01) which is in striking contrast to the 85.8% reduction in activity by 45 M PADK (p<0.0001) and the 87.3% reduced activity by 15 M CA074 (p<0.0001).

FIGS. 4A-4D: Protective effects assessed in hippocampal explants exhibiting synaptopathy induced by the acidotropic amine chloroquine (CQN) to experimentally model age-related lysosomal dysfunction. Hippocampal explants were maintained on culture inserts (FIG. 4A; size bar=3 mm) and staining with anti-synaptophysin (green) and DAPI (blue) show stable maintenance of native synaptic density in neuronal subfields after several weeks in culture (view-field width: 2.6 mm). Groups of stable, matured hippocampal explants (28-36/treatment) were treated daily with 60 M CQN for 3-6 days, while control slices were untreated for 6 days. The 0-6-day CQN samples consisted of 6-8 harvested slices each and were homogenized for protein determination and immunoblot assessment for the postsynaptic marker GluR1 and actin for a gel load control (FIG. 4B). Other groups of explants were treated with 60 M CQN for 5 or 6 days, followed by washout and infusion of vehicle, 10 μM PADK, or 10 μM SR-459 for two days. Immunoreactivity levels for GluR1 and other proteins (mean±SEM, n=3-6 per group) were assessed by unpaired t-tests: *p<0.05, **p<0.01 compared to vehicle-treated control; #p=0.03 compared to CQN+vehicle (70.2% recovery of GluR1). The postsynaptic protection by SR-459 included protected levels of the presynaptic marker synapsin II (syn II) and associated reduction in CQN-induced protein ubiquitination (FIG. 4C). Also produced by SR-459 was an associated increase in the LC3-II/LC3-I indicator of autophagy activation (FIG. 4D; ***p<0.001 vs. NT, ##p<0.01 compared to PADK).

FIGS. 5A-5D. Cognitive measures and synaptic markers were assessed in a model of mild cognitive impairment (MCI). Female Fischer rats of 12-13 months of age received 6-7 daily i.p. injections for 20 mg/kg/day of inactive ZFA compound, 15 mg/kg/day of SR-466, or 12 mg/kg/day of SR-459. Select groups of young (Y, 3 months old) and treated Fischer rats were tested with behavioral paradigms before rapid dissection. In those tested for passive avoidance, rats treated with SR-466 exhibited improved learning compared to ZFA-treated MCI rats indicated by enhanced avoidance of the shock area (FIG. 5A; mean±SEM). Exploratory habituation was assessed in an open field for monitoring movement, the young group exhibiting the largest percent reduction in exploring the same environment again on day two as compared to day one whereas the MCI group given control compound had the smallest reduction (FIG. 5B). Those MCI rats injected with SR compounds exhibited varying levels of improved exploratory habituation behavior. Note, equal mobility measures were found across the groups from initial minutes of each exploring period. Assessment of nest building/nestlet shredding (after providing a fresh nestlet pad on different days) was conducted since a deficit in this behavioral phenotype has been reported as a starting feature of neurological disorders. The MCI rats treated with SR-459 had evident improvement in nestlet scores compared to ZFA-treated rats (FIG. 5C). Equal aliquots of hippocampal homogenates prepared from the rat groups, including MCI rats treated with the natural product PanQ, were subjected to GluR1 immunostaining on separate immunoblots (FIG. 5D).

### DETAILED DESCRIPTION

The presently disclosed subject matter will now be described more fully. The presently disclosed subject matter can, however, be embodied in different forms and should not be construed as limited to the examples set forth herein below and in the accompanying Examples. Rather, these examples are provided so that this disclosure will be thorough and complete, and will fully convey the scope of the examples to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of time, concentration, dosage and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

As used herein, the terms “administration of” and/or “administering” a compound should be understood to refer to providing a compound of the presently disclosed subject matter to a subject in need of treatment.

As used herein, the terms “condition”, “disease condition”, “disease”, “disease state”, and “disorder” refer to physiological states in which diseased cells or cells of interest can be targeted with the compositions of the presently disclosed subject matter.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

The term “prevent” as used herein means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

A “subject” of analysis, diagnosis, or treatment is an animal. Such animals include mammals, in some examples, humans.

As used herein, the term “mammal” refers to any member of the class Mammalia, including, without limitation, humans, and nonhuman primates such as chimpanzees and other apes and monkey species; farm animals such as cattle, sheep, pigs, goats, and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some examples any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some examples the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, a “subject in need thereof” is a patient, animal, mammal, or human, who will benefit from the method of this presently disclosed subject matter.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. In some examples, the alkyl group is “lower alkyl.” “Lower alkyl” refers to an alkyl group having 1 to about 8 carbon atoms (i.e., a C₁₋₈ alkyl), e.g., 1, 2, 3, 4, 5, 6, 7, or 8 carbon atoms. In some examples, “lower alkyl” is C1-6 alkyl. In some examples, the alkyl is “higher alkyl.” “Higher alkyl” refers to an alkyl group having about 10 to about 20 carbon atoms, e.g., 10, 11, 12, 13, 14, 15, 16, 17, 18, 19, or 20 carbon atoms. In certain examples, “alkyl” refers, in particular, to C₁₋₈ or C₁₋₆ straight-chain alkyls and C₃₋₈ or C₃₋₆ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic moiety that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular examples, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, carbonyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to aryl groups wherein at least one atom of the backbone of the aromatic ring or rings is an atom other than carbon. Thus, heteroaryl groups have one or more non-carbon atoms selected from the group including, but not limited to, nitrogen, oxygen, and sulfur.

As used herein, the term “acyl” refers to an organic carboxylic acid group wherein the —OH of the carboxyl group has been replaced with another substituent (i.e., as represented by RC(═O)—, wherein R is an alkyl, substituted alkyl, aralkyl, substituted aralkyl, aryl or substituted aryl group as defined herein). As such, the term “acyl” specifically includes arylacyl groups, such as an acetylfuran and a phenacyl group. Specific examples of acyl groups include acetyl and benzoyl.

As used herein, the term “acetamide” can refer to a compound comprising the group —CHR—C(═O)—NR′R″, wherein R, R′ and R″ are independently selected from H, alkyl, substituted alkyl, aralkyl, substituted aralkyl, aryl, or substituted aryl, or wherein R′ and R″ together form a single divalent groups (e.g., an alkylene group), that, with the nitrogen atom of the acetamide form a nitrogen-containing heterocyclic ring structure.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The terms “heterocycle”, “heterocyclyl” “heterocycloalkyl” or “heterocyclic” refer to cycloalkyl groups (i.e., non-aromatic, cyclic groups as described hereinabove) wherein one or more of the backbone carbon atoms of a cyclic ring is replaced by a heteroatom (e.g., nitrogen, sulfur, or oxygen). Examples of heterocycles include, but are not limited to, tetrahydrofuran, tetrahydropyran, morpholine, dioxane, piperidine, piperazine, and pyrrolidine. Additional examples of heterocycles include, for example, the cyclic forms of sugars, such as ribose, glucose, galactose, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Alkoxyl” or “alkoxy” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

The terms “aryloxy” and “aryloxyl” refer to an aryl-O-group, wherein aryl is as previously described. The term “aryloxy as used herein can refer to, for example, phenoxy, p-chlorophenoxy, p-fluorophenoxy, p-methylphenoxy, p-methoxyphenoxy, and the like.

“Aralkyl” refers to an aryl-alkyl- group wherein aryl and alkyl are as previously described and include substituted aryl and substituted alkyl. Exemplary aralkyl groups include benzyl, phenylethyl, and naphthylmethyl. In some examples, the aromatic portion of the aralkyl group can be substituted by one or more aryl group substituents and/or the alkyl portion of the aralkyl group can be substituted by one or more alkyl group substituents and the aralkyl group can be a “substituted aralkyl” group.

The term “aralkylene” refers to a bivalent aliphatic group including both aliphatic and aromatic moieties. The aralkylene group can be straight, branched or cyclic. The aralkylene group also can be optionally substituted with one or more “alkyl group substituents” and/or one or more “aryl group substituents.” There can be optionally inserted along the aralkylene group one or more oxygen, sulfur or substituted or unsubstituted nitrogen atoms.

The term “amino” refers to the —NR′R″ group, wherein R′ and R″ are each independently selected from the group including H and substituted and unsubstituted alkyl, cycloalkyl, heterocycle, aralkyl, aryl, and heteroaryl. In some examples, the amino group is —NH₂.

The terms “alkylamino” and “aminoalkyl” refer to a —NHR group where R is alkyl or substituted alkyl. The term “arylamino” refers to a —NHR group where R is aryl or substituted aryl.

The term “carbonyl” refers to the —(C═O)— or a double bonded oxygen substituent attached to a carbon atom of a previously named parent group.

The terms “carboxylate” and “carboxylic acid” can refer to the groups —C(═O)—O⁻ and —C(═O)—OH, respectively. In some examples, “carboxylate” can refer to either the —C(═O)—O⁻ or —C(═O)—OH group. In some examples, the term “carboxyl” can also be used to refer to a carboxylate or carboxylic acid group.

The terms “halo”, “halide”, or “halogen” as used herein refer to fluoro, chloro, bromo, and iodo groups.

The term “perhaloalkyl” refers to an alkyl group wherein all of the hydrogen atoms are replaced by halo. Thus, for example, perhaloalkyl can refer to a “perfluroalkyl” group wherein all of the hydrogen atoms of the alkyl group are replaced by fluoro. Perhaloalkyl groups include, but are not limited to, —CF₃.

The terms “hydroxyl” and “hydroxy” refer to the —OH group.

The term “oxo” refers to a compound described previously herein wherein a carbon atom is replaced by an oxygen atom.

The term “thio” refers to the —S— or —SH group.

The terms “alkylthio” and “thioalkyl” refer to a —SR group where R is alkyl or substituted alkyl. The term “arylthiol” refers to a —SR group where R is aryl or substituted aryl.

The term “cyano” refers to the —CN group.

The term “nitro” refers to the —NO₂ group.

A line crossed by a wavy line, e.g., in the structure:

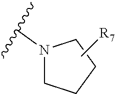

indicates the site where the indicated substituent can bond to another group.

### II. Brain Protein Accumulation Diseases

As used herein “protein accumulation diseases” are diseases that are associated with protein accumulation and aggregation. “Brain protein accumulation diseases” are associated with protein accumulation and aggregation in the brain. For example, Alzheimer's disease involves accumulation of the A3 protein, Parkinson's disease involves accumulation of α-synuclein, Huntington's disease involves aggregation of mutated huntingtin proteins, and amyotrophic lateral sclerosis involves accumulation of mutated superoxide dismutase-1 proteins. Some chronic psychiatric disorders, such as schizophrenia, bipolar disorder, and recurrent major depression, have also been associated with protein aggregation. The reduction of protein accumulation events is important for slowing the progression of these diseases and disorders. Studies indicate that protein degradation processes that clear these aggregated proteins could provide treatment for some or all of these diseases and disorders.

As used herein, “Mild Cognitive Impairment” or “MCI” means an impairment in memory that is not severe enough to affect daily function and that is linked to a condition other than AD, other than Parkinson's disease, and other than Huntington's disease. MCI can comprise deficits in cognitive functions in addition to impaired memory. Subjects with MCI have actual memory loss, rather than the sometimes slow memory retrieval from relatively preserved memory storage in age-matched controls. MCI tends to first affect short-term (also called episodic) memory first. Subjects have trouble remembering recent conversations, the location of commonly used items, and appointments. However, memory for remote events is typically intact, as is attention (also called working memory-subjects can repeat lists of items and do simple calculations). MCI often progresses to dementia. MCI causes greater memory loss than age-associated memory impairment; memory and sometimes other cognitive functions are worse in subjects with this disorder than in age-matched controls, but daily functioning is not affected. In contrast, dementia impairs daily functioning.

As used herein, “dementia” means memory loss plus evidence of cognitive and behavioral dysfunction. A subject with dementia may have difficulty with finding words and/or naming objects (aphasia), doing previously learned motor activities (apraxia), or planning and organizing everyday tasks, such as meals, shopping, and bill paying (impaired executive function). A subject's personality may change; for example, the subject can become uncharacteristically irritable, anxious, agitated, and/or inflexible.

Clinical criteria for dementia include cognitive or behavioral (neuropsychiatric) symptoms that interfere with the ability to function at work or do usual daily activities. These symptoms represent a decline from previous levels of functioning. These symptoms are not explained by delirium or a major psychiatric disorder. In some examples, the cognitive or behavioral impairment involves ≥2 of the following domains: i) Impaired ability to acquire and remember new information (amnesia), ii) Language dysfunction (aphasia), iii) Visuospatial dysfunction (agnosia; e.g., inability to recognize faces or common objects), iv) Impaired executive function, including reasoning, handling of complex tasks, and/or judgment (apraxia); v) Changes in personality, behavior, or comportment.

Dementia impairs cognition globally. Onset is gradual, although family members may suddenly notice deficits (e.g., when function becomes impaired). Often, loss of short-term memory is the first sign. Although symptoms of dementia exist in a continuum, they can be divided into early, intermediate, and late. Personality changes and behavioral disturbances may develop early or late. Motor and other focal neurologic deficits occur at different stages, depending on the type of dementia. Incidence of seizures is somewhat increased during all stages. Psychosis-hallucinations, delusions, or paranoia-occurs in about 10% of subjects with dementia, although a higher percentage may experience these symptoms temporarily.

During “early dementia” recent memory is impaired; learning and retaining new information become difficult. Language problems (especially with word finding), mood swings, and personality changes develop. Subjects may have progressive difficulty with independent activities of daily living (e.g., balancing their checkbook, finding their way around, remembering where they put things). Abstract thinking, insight, or judgment may be impaired. Subjects can respond to loss of independence and memory with irritability, hostility, and agitation. Functional ability may be further limited by the following:
Agnosia: Impaired ability to identify objects despite intact sensory function.
Apraxia: Impaired ability to do previously learned motor activities despite intact motor function.
Aphasia: Impaired ability to comprehend or use language.

Although early dementia may not compromise sociability, family members may report strange behavior accompanied by emotional lability. In some examples, early dementia is linked to a condition other than AD, other than Parkinson's disease and other than Huntington's disease.

During “intermediate dementia” subjects become unable to learn and recall new information. Memory of remote events is reduced but not totally lost. Subjects may require help with basic activities of daily living (e.g., bathing, eating, dressing, toileting). Personality changes may progress. Subjects can become irritable, anxious, self-centered, inflexible, or angry more easily, or they can become more passive, with a flat affect, depression, indecisiveness, lack of spontaneity, or general withdrawal from social situations. Behavior disorders can develop: subjects may wander or become suddenly and inappropriately agitated, hostile, uncooperative, or physically aggressive.

By this stage, subjects have lost all sense of time and place because they cannot effectively use normal environmental and social cues. Subjects often get lost; they may be unable to find their own bedroom or bathroom. They often remain ambulatory but are at risk of falls or accidents secondary to confusion. Altered sensation or perception may culminate in psychosis with hallucinations and paranoid and persecutory delusions. Sleep patterns are often disorganized.

During “late dementia” subjects cannot walk, feed themselves, or do any other activities of daily living; they may become incontinent. Recent and remote memory is completely lost. Subjects may be unable to swallow. They are at risk of undernutrition, pneumonia (especially due to aspiration), and pressure ulcers. Because they depend completely on others for care, placement in a long-term care facility often becomes necessary. Eventually, subjects become mute. End-stage dementia results in coma and death, usually due to infection.

Cytoplasmic deposition of α-synuclein is a common pathological feature of many neurodegenerative diseases collectively called α-synucleinopathies, including familial Parkinson's disease.

Parkinson's disease (Parkinson's) is a motor system disorder which is associated with the loss of dopamine-producing brain cells. Dopamine is necessary for coordinated muscle function and movement. Dopamine is normally produced by certain nerve cells (neurons) in the substantia nigra region of the brain; however, Parkinson's subjects experience a loss of these neurons which leads to impaired movement. This loss of neurons is associated with the accumulation of α-synuclein, a protein that is mutated and/or misfolded in Parkinson's and other diseases. The α-synuclein forms aggregates that accumulate in Lewy bodies, and which are seen in the brains of subjects who have died from Parkinson's.

The age-related neurodegenerative disorder Alzheimer's disease (AD) involves the accumulation of oligomeric species, protein aggregation, and altered brain function. One of the major hallmarks of AD is the plaque deposits consisting primarily of amyloid fibrils formed by the amyloid beta peptide Aβ1-42 as well as the buildup of soluble oligomers of this peptide. Mutations associated with familial AD, including mutations in the amyloid precursor protein (APP), strongly implicate Aβ1-42 as a causative factor since the mutations increase the relative amount of this Aβ peptide. Increased Aβ is one of the earliest events in AD, and, besides extracellular accumulation, Aβ oligomerization also occurs intraneuronally. Aβ oligomers disrupt synaptic plasticity, impair synaptic responses and memory, and cause cytotoxicity, as well as produce synaptic deterioration. Aβ oligomers, especially trimers and multiples of trimeric species, are particularly stable.

Two classes of drugs approved for treating AD include acetyl-cholinesterase inhibitors and N-methyl-D-aspartic acid (NMDA) receptor antagonists. Both types of drugs only affect the symptoms of AD. Acetyl-cholinesterase inhibitors are for mild to moderate AD and have modest effects in a small percentage of subjects who take the drug and are typically ineffective after 6-12 months of use. The NMDA receptor antagonist that is available treats the secondary pathology but not the protein accumulation in mild to severe AD.

The neurodegenerative disorder Huntington's disease (Huntington's) is caused by a trinucleotide repeat expansion in the huntingtin gene which codes for huntingtin protein, “Htt.” People who have Huntington's disease have more C-A-G codons on their huntingtin gene which results in Htts that are “altered” or abnormal in that they have an excess number of glutamines. As a result of the excess glutamines, these altered Htts form protein aggregates which can interfere with nerve cell function.

Amyotrophic lateral sclerosis (ALS) is a progressive neurodegenerative disease that attacks nerve cells in the brain and the spinal cord. Neuronal cell death has been linked to the presence of aggregates of mutant superoxide dismutase-1 (SOD1) protein. Mutant SOD1 accumulates to form high molecular weight amorphous aggregates which can interact with other proteins. When these mutant SOD1 proteins accumulate and form aggregates in a neuronal cell, the cell almost always dies.

Risk Factors from Genetic Background and Life Experiences

As described hereinabove, AD is characterized by progressive loss of cognitive function and is one of a class of disorders, including many other types of dementias or associated diseases, referred to as protein accumulation disorders. These diseases and disorders are believed to involve imbalances between protein production and protein clearance. In addition to age, other factors, have been linked to AD risk.

Over a hundred gene mutations have been identified that, together, account for the 4-5% of AD cases that are hereditary (typically early-onset AD). The vast majority of AD is what is considered sporadic, often called late-onset AD (LOAD). Many sporadic AD cases are carriers of the APOE4 allele. APOE4 is the strongest risk factor gene for AD, but it does not cause AD in all carriers. Some drugs in development target APOE4 carriers with early stage AD.

Certain life experiences can also increase the risk of AD. Reports indicate that people that have had a stroke, experienced seizures, or have been exposed to military blasts have a higher prevalence of AD. There have been several reports of evidence that toxin exposures (military-related, pesticides, pollution-related) cause synaptic compromise similar to that which occurs with aging and AD. Thus, these exposures represent risk factors that can influence the likelihood of developing dementia later in life. Similarly, military blast exposures elicit a unique array of synaptic disturbances in brain tissue (see Almeida et al. 2021 Brain Pathology 31:e12936). In addition, the blast-induced reduction in the synaptic marker NCAM180 was found to correlate with pathogenic phosphorylated tau levels measured with the AT8 antibody (R=−0.667; p<0.01), which also labeled tau pathology in stochastic patterns of selective neurons. The findings reveal that detonations of military explosives produce distinct synaptopathy in a brain region involved in learning, emotional-laden memories, and social behavior, perhaps explaining recurring episodes of depression, anxiety, and attention problems in many veterans.

Lastly, in addition to the blast-induced tauopathy, another indication of protein accumulation pathology is the increase in ubiquitinated proteins in blast-exposed brain tissue. This indicates compromise of the ubiquitin-proteasome system which plays an important role in protein quality control and homeostasis for cellular health.

### Cathepsin B

Cathepsin B has been reported to degrade Aβ42 and has also been shown to promote clearance of α-synuclein, a protein found accumulated in several neurodegenerative disorders including AD and Parkinson's disease (PD). Using animal models of AD, MCI, and PD, published evidence indicates that such drugs reduce protein accumulation pathology in correspondence with synaptic restoration and improved measures of learning and memory. Further, it has been reported that lysosomal alterations likely contribute to age-related dysfunction in autophagic lysosomal processing, which can include impairment in organelle maturation and fusion with autophagosomes. See Hwang et al. 2019, Int. J. Mol. Sci., 20, 4432. Accordingly, it appears that the lysosomal pathway is a target in a broad disease-modifying approach to treat AD, PD, and MCI. Early work on a prototype molecule that enhances Cathepsin B maturation also found that such drug action had a positive influence on the ubiquitin-proteasome system (see Farizatto et al. 2017 PLoS ONE 12: e0182895).

In some examples, the presently disclosed subject matter is related to the development of a new classes of therapeutic agents that target the earliest steps of the AD pathogenic cascade, which includes: i) disturbances in protein clearance pathways, ii) the buildup of corrupted protein structures/aggregates, iii) destabilization of microtubules and their transport mechanisms, iv) disruption of transport processes needed for synaptic maintenance, and v) progressive loss of cortical synapses—such loss being a well-known correlate of cognitive decline in AD and aging. In some examples, the presently disclosed subject matter provides compounds to reduce AD-type pathology by enhancing the protein clearance capacity of the autophagy-lysosomal pathway (a major contributor to proteostasis and cellular maintenance), e.g., through the drug action of positive modulation and improved maturation efficiency of the lysosomal enzyme cathepsin B and associated activation of autophagy. According to one aspect, the presently disclosed subject matter is based on the hypothesis that drugs that act through Cathepsin B maturation can have a dual effect on two major protein clearance pathways affected by AD and other dementias, i.e., autophagy-lysosomal pathway and the ubiquitin-proteasome system.

CatB has also been implicated in the degradation of α-synuclein species for a therapeutic strategy for Parkinson's disease and other synucleinopathies (Hwang et al. 2019, Int. J. Mol. Sci., 20, 4432). Improving active CatB in different in vitro and in vivo models of Alzheimer's pathogenesis was associated with reduced levels of Aβ42 peptide and Aβ42 deposits (Butler et al. (2011) PLoS ONE 6:e20501; Hwang et al. 2019, Int. J. Mol. Sci., 20, 4432).

Also, the targeting of the progranulin (PGRN)-prosaposin (PSAP) complex can enhance lysosomal function in neurodegenerative diseases, such as Progressive Supranuclear Palsy (PSP) and broader tauopathies, including Alzheimer's and Parkinson's diseases. See Verwaerde, P. et al., ChemMedChem. 2025 Mar. 27:e202400891.

### III. Compounds and Pharmaceutical Compositions for the Positive Modulation of the Autophagy-Lysosomal Pathway

In some examples, the presently disclosed subject matter provides new classes of compounds, referred to herein as “SR” compounds, which increase levels of the mature 30-kDa cathepsin B (CatB30), like PADK and some natural products. However, the presently disclosed SR compounds provide a different CatB30/CatB25 ratio than these other agents that increase CatB30, indicating a possible effect on protease maturation efficiency. Of particular note, the SR compounds described herein activate the autophagy-lysosomal pathway (as determined by assaying the LC3II/I ratio) significantly better than PADK. As would be apparent to one of ordinary skill in the art upon a review of the instant disclosure, reference to a formula or to a compound refers to racemic forms of the formula or compound, as well as any and all particular racemate forms of the compound. In some examples, particular racemate forms of a compound are recited.

In some examples, the SR compound comprises a substituted 1,3-oxazin-4-one moiety or an analog thereof or a substituted benzopyrone moiety or an analog thereof. The term “analog” as used herein refers in some examples to compounds where an oxygen atom in the backbone of a ring is replaced by an alkylene (e.g., methylene) group or another heteroatom, where a nitrogen atom is replaced by a carbon atom, where a carbon-carbon double bond is replaced by a carbon-carbon single bond, or where a —C(═O)— group is replaced by a —CH(OH)—, —CH(OR)— group (where R is alkyl, aralkyl, or aryl), or a —CH₂— (e.g., methylene) group. In some examples, the SR compound comprises a 1,3-oxazin-4-one or benzopyrone moiety or analog thereof that is substituted with 1, 2, or 3 substituents.

In some examples, the SR compound has a structure of Formula (I) or Formula (II):

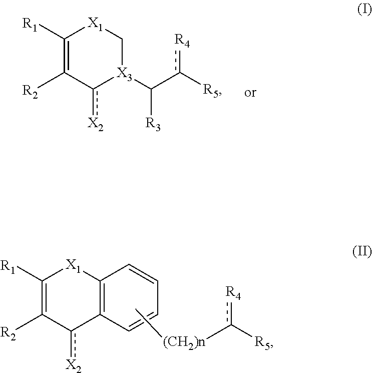

wherein: n is 0, 1, 2, 3, 4, 5, or 6; each [symbol] is independently a double or single bond; X₁ is O or methylene; X₂ is H, OH, SH, NH₂, O, S, or NH, subject to the proviso that when the [symbol] [symbol] to which X₂ is attached is a single bond, X₂ is selected from H, OH, SH, and NH₂ and when the [symbol] to which X₂ is attached is a double bond, X₂ is O, S or NH; X₃ is CH or N; R₁, R₂, and R₃ are independently selected from the group comprising H, OH, alkyl, substituted alkyl, alkoxy, aralkyl, substituted aralkyl, aryl, and substituted aryl; R₄ is OH or O, subject to the proviso that R₄ is OH when the [symbol] to which R₄ is attached is a single bond and O when the [symbol] to which R₄ is attached is a double bond; and R₅ is selected from H, OH, alkyl, substituted alkyl, aralkyl, substituted aralkyl, aryl, substituted aryl, and N(R₆)₂, wherein each R₆ is independently selected from H, acyl, alkyl, substituted alkyl, aralkyl, substituted aralkyl, aryl, and substituted aryl, or wherein the two R₆ together form a substituted or unsubstituted alkylene or aralkylene group; or a pharmaceutically acceptable salt thereof.

In some examples, one of R₁ and R₂ is H or OH and one of R₁ and R₂ is aralkyl, substituted aralkyl, aryl, or substituted aryl. In some examples, one or R₁ and R₂ is H and one or R₁ and R₂ is aralkyl, substituted aralkyl, aryl, or substituted aryl. In some examples, one of R₁ and R₂ is phenyl or naphthyl. In some examples, one of R₁ and R₂ is substituted phenyl (e.g., hydroxy-substituted phenyl). In some examples, both R₁ and R₂ are H.

In some examples, the compound comprises a substituted 1,3-oxazin-4-one or analog thereof. Thus, in some examples, the compound has a structure of Formula (I):

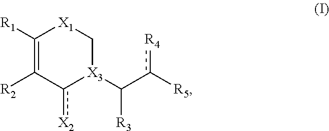

wherein: each [symbol] is independently a double or single bond; X₁ is O or methylene; X₂ is H, OH, SH, NH₂, O, S, or NH, subject to the proviso that when the [symbol] to which X₂ is attached is a single bond, X₂ is selected from H, OH, SH, and NH₂ and when the [symbol] to which X₂ is attached is a double bond, X₂ is O, S or NH; X₃ is CH or N; R₁, R₂, and R₃ are independently selected from the group comprising H, OH, alkyl, substituted alkyl, alkoxy, aralkyl, substituted aralkyl, aryl, and substituted aryl; R₄ is OH or O, subject to the proviso that R₄ is OH when the [symbol] to which R₄ is attached is a single bond and O when the [symbol] to which R₄ is attached is a double bond; and R₅ is selected from H, OH, alkyl, substituted alkyl, aralkyl, substituted aralkyl, aryl, substituted aryl, and N(R₆)₂, wherein each R₆ is independently selected from H, acyl, alkyl, substituted alkyl, aralkyl, substituted aralkyl, aryl, and substituted aryl, or wherein the two R₆ together form a substituted or unsubstituted alkylene or aralkylene group; or a pharmaceutically acceptable salt thereof.

In some examples, the compound of Formula (I) has a structure of Formula (Ia):

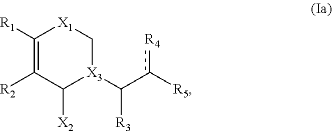

wherein: [symbol] is a double or single bond; X₁ is O or methylene; X₂ is H or OH; X₃ is CH or N; R₁, R₂, and R₃ are independently selected from the group comprising H, alkyl, substituted alkyl, aralkyl, substituted aralkyl, aryl, and substituted aryl; R₄ is OH or O, subject to the proviso that R₄ is OH when the [symbol] to which R₄ is attached is a single bond and O when the [symbol] to which R₄ is attached is a double bond; and R₅ is selected from H, OH, alkyl, substituted alkyl, aralkyl, substituted aralkyl, aryl, substituted aryl, and N(R₆)₂, wherein each R₆ is independently selected from H, acyl, alkyl, substituted alkyl, aralkyl, substituted aralkyl, aryl, and substituted aryl, or wherein the two R₆ together form a substituted or unsubstituted alkylene or aralkylene group; or a pharmaceutically acceptable salt thereof. In some examples, X₁ is O. In some examples, X₂ is H. In some examples, R₃ is aralkyl (e.g., benzyl). In some examples, [symbol] is a single bond and R₄ is OH. In some examples, one of R₁ and R₂ is aryl (e.g., phenyl) or substituted aryl (e.g., substituted phenyl) and one of R₁ and R₂ is H. In some examples, R₅ is substituted alkyl. In some examples, R₅ is amino-substituted alkyl. In some examples, the amino-substituted alkyl is —CH₂—N(R₉)(R₁₀), where R₉ and R₁₀ are independently selected from H, alkyl, substituted alkyl, aralkyl, substituted aralkyl, aryl, substituted aryl, and —C(═O)R₁₁, wherein R₁₁ is selected from alkyl, substituted alkyl, aralkyl, substituted aralkyl, aryl, and substituted aryl. In some examples, R₉ is —C(═O)R₁₁, R₁₀ is aralkyl (e.g., benzyl), and R₁₁ is alkyl (e.g., methyl).

In some examples, the compound of Formula (I) has a structure of Formula (Ib):

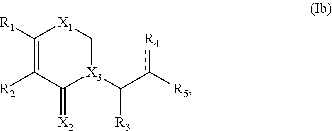

wherein [symbol] is a double or single bond; X₁, X₃, R₁, R₂, R₃, R₄, and R₅ are as defined for Formula (I) and were X₂ is selected from O, S, and NH. In some examples, X₂ is O. In some examples, X₁ is O and/or X₃ is N.

In some examples, X₁ and X₂ are each O. In some examples, X₁ and X₂ are each O and X₃ is N and the compound of Formula (Ib) has a structure of Formula (III):

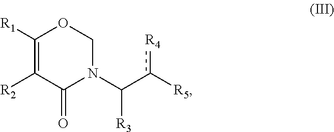

wherein [symbol] is a double or single bond and R₁, R₂, R₃, R₄, and R₅ are as defined for Formula (I). In some examples, the compound of Formula (III) has a structure of Formula (IIIa):

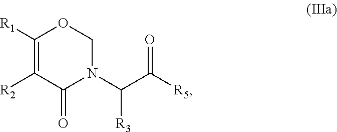

wherein R₁, R₂, R₃, and R₅ are as defined for Formula (I). In some examples, R₅ is selected from OH, lower alkyl (e.g., methyl, ethyl, n-propyl, isopropyl, n-butyl, sec-butyl, tert-butyl, isopentyl, etc.) and N(R₆)₂. In some examples, R₅ is N(R₆)₂ and the two R₆ together form a substituted or unsubstituted 4-, 5-, 6-, 7-, or 8-membered ring. “Substituted” here is meant to encompass all the definitions presented herein for the term “substituted”, including but not limited to “alkyl group substituent”, “aryl group substituent”, “substituted alkyl”, and “substituted aryl”, as set forth elsewhere herein. Thus, there can be optionally inserted along the 4-, 5-, 6-, 7-, or 8-membered ring one or more oxygen, sulfur or substituted or unsubstituted nitrogen atoms. In some examples, R₅ is

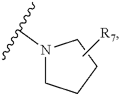

wherein R₇ is selected from H and C(═O)R₈, wherein R₈ is selected from alkyl (e.g., lower alkyl) and substituted alkyl (e.g., substituted lower alkyl). In some examples, R₈ is methyl.

In some examples, the compound of Formula (III) has a structure of Formula (IIIb):

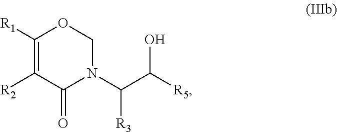

wherein R₁, R₂, R₃, and R₅ are as defined for Formula (I). In some examples, R₃ is H, benzyl, or substituted benzyl. In some examples, R₃ is benzyl. In some examples, R₅ is amino-substituted alkyl (e.g., amino-substituted lower alkyl). In some examples, R₅ is —CH₂—N(R₉)(R₁₀), wherein R₉ and R₁₀ are independently selected from H, alkyl, substituted alkyl, aralkyl, substituted aralkyl, aryl, substituted aryl, and —C(═O)R₁₁, wherein R₁₁ is selected from alkyl, substituted alkyl, aralkyl, substituted aralkyl, aryl, and substituted aryl. In some examples, R₉ is substituted or unsubstituted aralkyl and/or R₁₀ is —C(═O)R₁₁. In some examples, R₉ is —CH₂—R₁₂, wherein R₁₂ is substituted or unsubstituted phenyl, naphthyl, or pyridine and/or R₁₀ is —C(═O)R₁₁, wherein R₁₁ is alkyl or substituted alkyl (e.g., methyl).

In some examples, the compound of Formula (I) is selected from the group comprising:

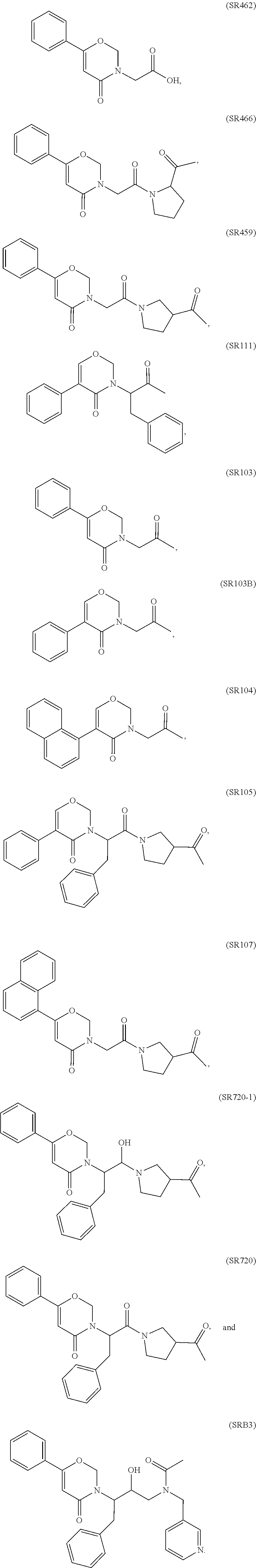

In some examples, the compound is selected from the group comprising SR466, SR462, and SR459. In some examples, the compound is SR459 or SR466, or a stereoisomer form thereof. In some examples, the compound is:

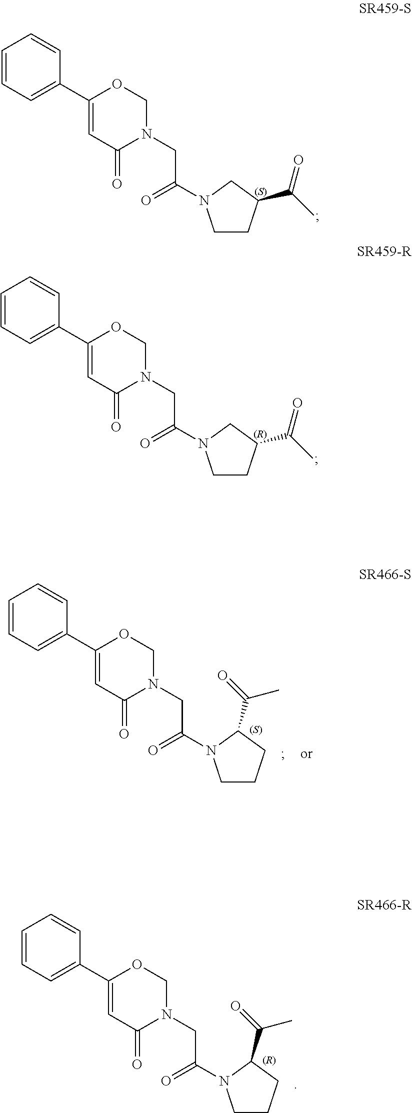

See the Examples hereinbelow.

In some examples, the compound is:

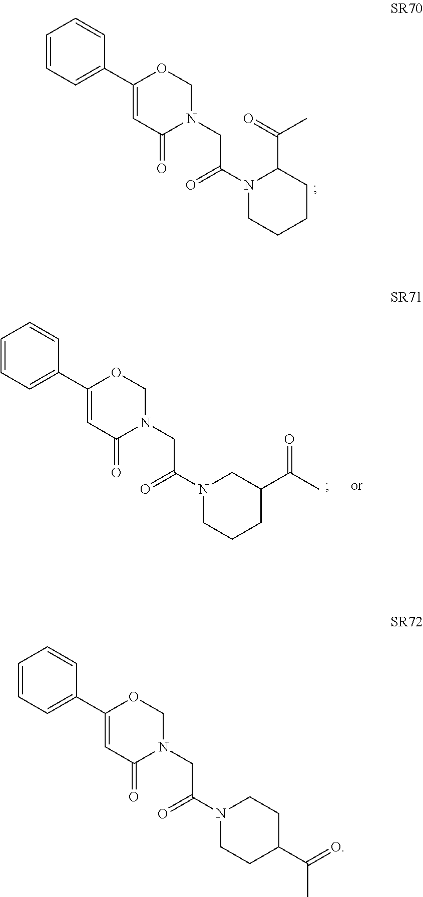 [SMILES: CC(=O)C1CCCCN1C(=O)CN1COC(c2ccccc2)=CC1=O ; CC(=O)C1CCCN(C(=O)CN2COC(c3ccccc3)=CC2=O)C1 ; CC(=O)C1CCN(C(=O)CN2COC(c3ccccc3)=CC2=O)CC1]

See the Examples herein below.

In some examples, the compound is:

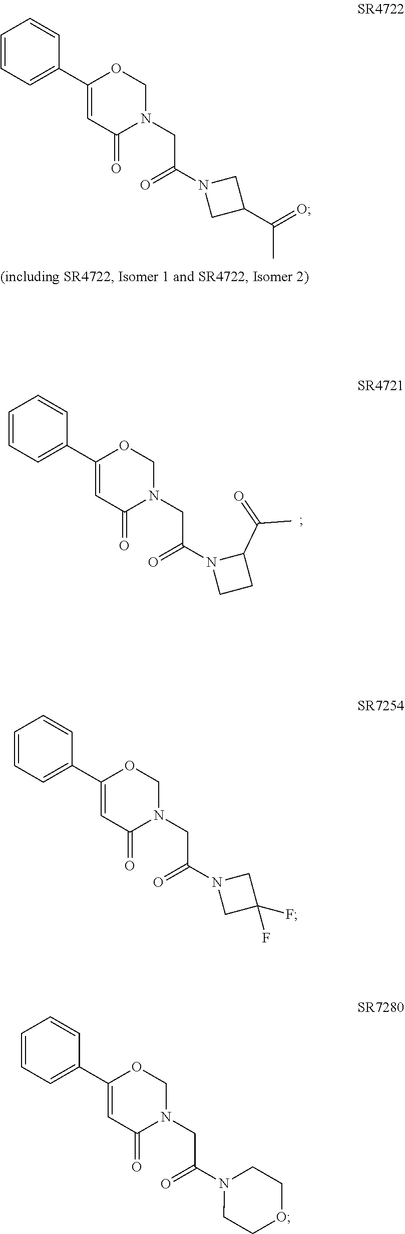 [SMILES: CC(=O)C1CN(C(=O)CN2COC(c3ccccc3)=CC2=O)C1 ; CC(=O)C1CCN1C(=O)CN1COC(c2ccccc2)=CC1=O ; O=C1C=C(c2ccccc2)OCN1CC(=O)N1CC(F)(F)C1 ; O=C1C=C(c2ccccc2)OCN1CC(=O)N1CCOCC1]

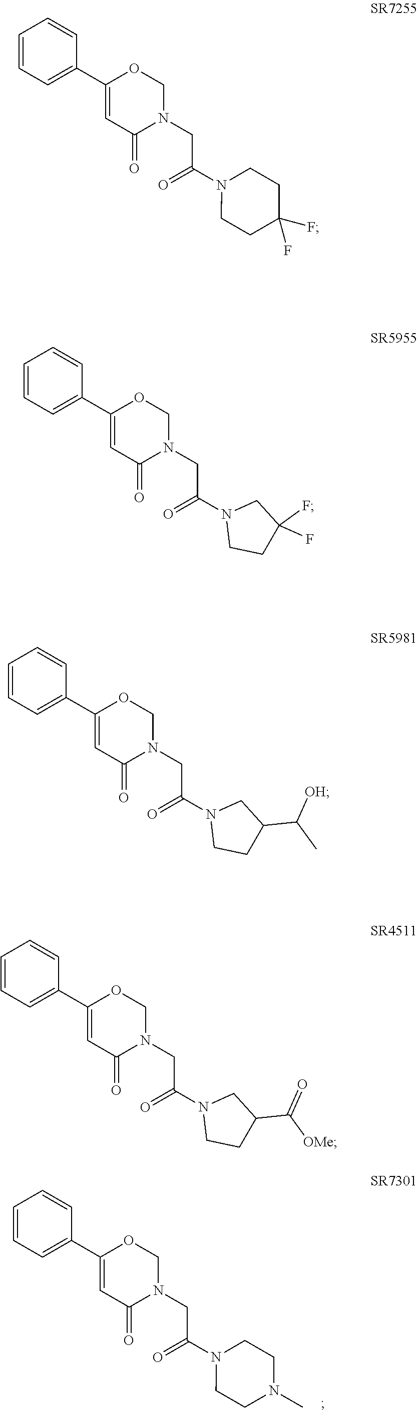 [SMILES: O=C1C=C(c2ccccc2)OCN1CC(=O)N1CCC(F)(F)CC1 ; O=C1C=C(c2ccccc2)OCN1CC(=O)N1CCC(F)(F)C1 ; CC(O)C1CCN(C(=O)CN2COC(c3ccccc3)=CC2=O)C1 ; COC(=O)C1CCN(C(=O)CN2COC(c3ccccc3)=CC2=O)C1 ; CN1CCN(C(=O)CN2COC(c3ccccc3)=CC2=O)CC1]

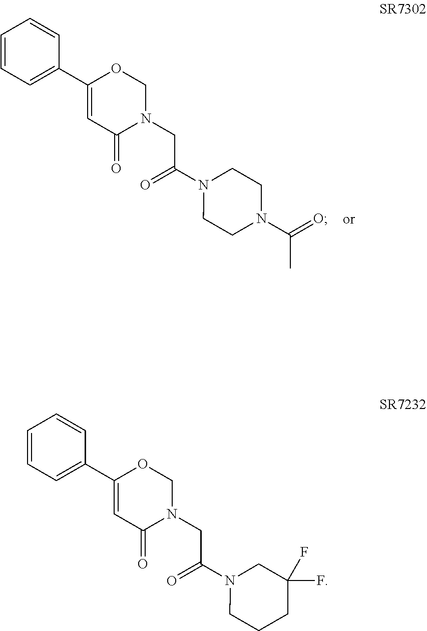 [SMILES: CC(=O)N1CCN(C(=O)CN2COC(c3ccccc3)=CC2=O)CC1 ; O=C1C=C(c2ccccc2)OCN1CC(=O)N1CCCC(F)(F)C1]

Guidance for preparing these compounds can be found elsewhere herein, including the 10 EXAMPLES presented below.

In some examples, the compound comprises a benzopyrone moiety or an analog thereof. In some examples, the compound has a structure of Formula (II):

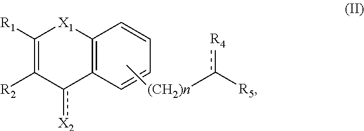

wherein: n is 0, 1, 2, 3, 4, 5, or 6; each [symbol] is independently a double or single bond; X₁ is O or methylene; X₂ is H, OH, SH, NH₂, O, S, or NH, subject to the proviso that when the [symbol] [symbol] to which X₂ is attached is a single bond, X₂ is selected from H, OH, SH, and NH₂ and when the [symbol] to which X₂ is attached is a double bond, X₂ is O, S or NH; X₃ is CH or N; R₁ and R₂ are independently selected from the group comprising H, OH, alkyl, substituted alkyl, alkoxy, aralkyl, substituted aralkyl, aryl, and substituted aryl; R₄ is OH or O, subject to the proviso that R₄ is OH when the [symbol] to which R₄ is attached is a single bond and O when the [symbol] to which R₄ is attached is a double bond; and R₅ is selected from H, OH, alkyl, substituted alkyl, aralkyl, substituted aralkyl, aryl, substituted aryl, and N(R₆)₂, wherein each R₆ is independently selected from H, acyl, alkyl, substituted alkyl, aralkyl, substituted aralkyl, aryl, and substituted aryl, or wherein the two R₆ together form a substituted or unsubstituted alkylene or aralkylene group; or a pharmaceutically acceptable salt thereof. In some examples, the compound of Formula (II) has a structure of Formula (IIa):

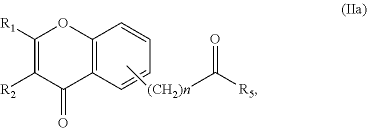

wherein n, R₁, R₂, and R₅ are as defined for Formula (II).

In some examples, n is 1. In some examples, R₅ is

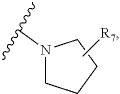

wherein R₇ is selected from H and —C(═O)R₈, wherein R₈ is selected from alkyl (e.g., lower alkyl) and substituted alkyl (e.g., substituted lower alkyl). In some examples, R₈ is methyl.

In some examples, the compound of Formula (II) is selected from:

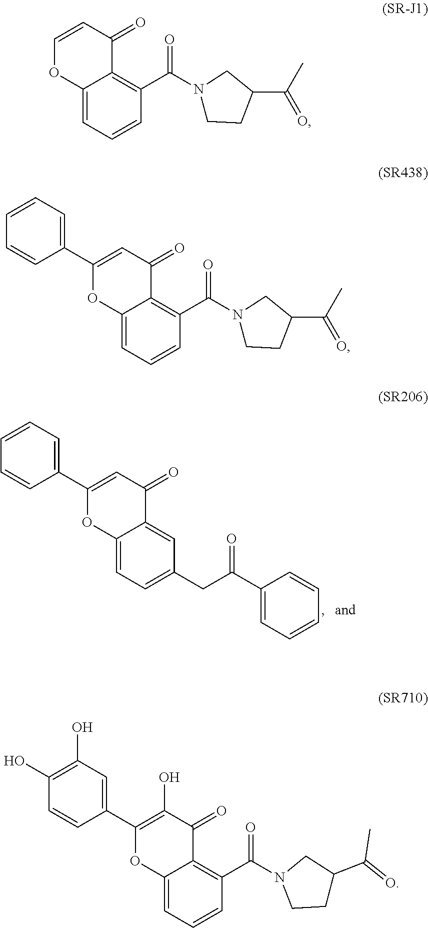 [SMILES: CC(=O)C1CCN(C(=O)c2cccc3occc(=O)c23)C1 ; CC(=O)C1CCN(C(=O)c2cccc3oc(-c4ccccc4)cc(=O)c23)C1 ; O=C(Cc1ccc2oc(-c3ccccc3)cc(=O)c2c1)c1ccccc1 ; CC(=O)C1CCN(C(=O)c2cccc3oc(-c4ccc(O)c(O)c4)c(O)c(=O)c23)C1]

Additional examples of SR438 related compounds include:

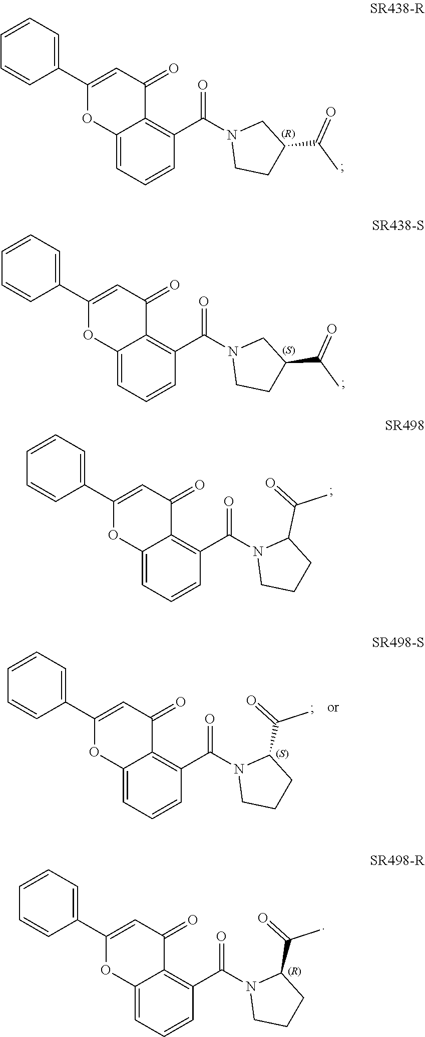

Guidance for preparing these compounds can be found elsewhere herein, including the EXAMPLES presented below.

In some examples, the compound is:

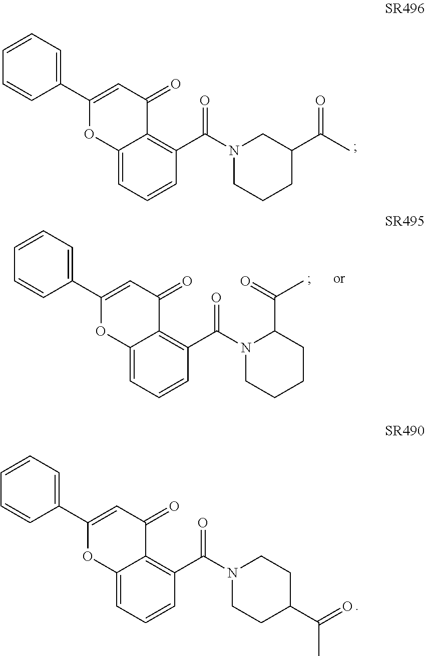 [SMILES: CC(=O)C1CCCN(C(=O)c2cccc3oc(-c4ccccc4)cc(=O)c23)C1 ; CC(=O)C1CCCCN1C(=O)c1cccc2oc(-c3ccccc3)cc(=O)c12 ; CC(=O)C1CCN(C(=O)c2cccc3oc(-c4ccccc4)cc(=O)c23)CC1]

Guidance for preparing these compounds can be found elsewhere herein, including the Examples presented below.

In some examples, the compound is:

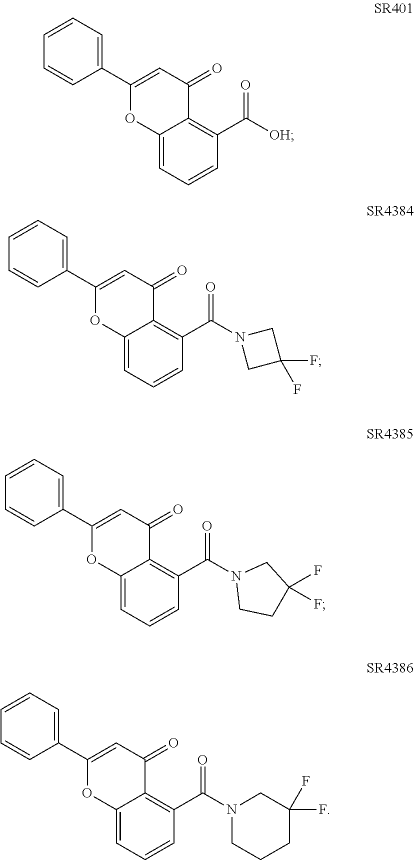 [SMILES: O=C(O)c1cccc2oc(-c3ccccc3)cc(=O)c12 ; O=C(c1cccc2oc(-c3ccccc3)cc(=O)c12)N1CC(F)(F)C1 ; O=C(c1cccc2oc(-c3ccccc3)cc(=O)c12)N1CCC(F)(F)C1 ; O=C(c1cccc2oc(-c3ccccc3)cc(=O)c12)N1CCCC(F)(F)C1]

Guidance for preparing these compounds can be found elsewhere herein, including the Examples presented below.

In some examples, a compound of the presently disclosed subject matter is one of:

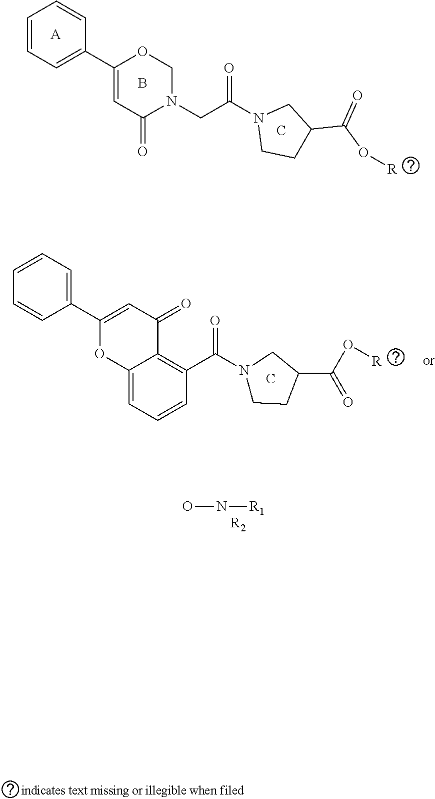

Looking at the above-presented examples, with representative, non-limiting designated A B and C ring moieties, the C-ring is a 4-, 5- (as pictured), 6-, 7-, or 8-member ring, which can be substituted or unsubstituted, e.g. a substituted or unsubstituted alkylene or aralkylene group. “Substituted” here is meant to encompass all the definitions presented herein for the term “substituted”, including but not limited to “alkyl group substituent”, “aryl group substituent”, “substituted alkyl”, and “substituted aryl”, as set forth elsewhere herein. Thus, there can be optionally inserted along the 4-, 5-, 6-, 7-, or 8-membered ring one or more oxygen, sulfur or substituted or unsubstituted nitrogen atoms. In some examples, the side group coming off the C-ring of both structures can include a methyl ester change in addition to methyl ketone chemistry also described herein. The compounds can include branching off with an amide functional group. In the examples presented above, R, R₁, and R₂ can include but are not limited to H, acyl, alkyl, substituted alkyl, aralkyl, alkoxy, substituted aralkyl, aryl, or substituted aryl, or wherein R₁, and R₂ together form a substituted or unsubstituted alkylene or aralkylene group. Guidance for preparing these compounds can be found elsewhere herein, including the Examples presented below.

The following IUPAC nomenclature is provided for compounds of the presently disclosed subject matter and other compounds disclosed herein to facilitate description of the compounds:
SR70 is 3-[2-(2-Acetyl-1-piperidyl)-2-oxoethyl]-6-phenyl-2,3-dihydro-4H-1,3-oxazin-4-one
SR71 is 3-[2-(3-Acetyl-1-piperidyl)-2-oxoethyl]-6-phenyl-2,3-dihydro-4H-1,3-oxazin-4-one
SR72 is 3-[2-(4-Acetyl-1-piperidyl)-2-oxoethyl]-6-phenyl-2,3-dihydro-4H-1,3-oxazin-4-one
SR103 is 3-Acetonyl-6-phenyl-2,3-dihydro-4H-1,3-oxazin-4-one
SR103B is 3-Acetonyl-5-phenyl-2,3-dihydro-4H-1,3-oxazin-4-one
SR104 is 3-Acetonyl-5-(1-naphthyl)-2,3-dihydro-4H-1,3-oxazin-4-one
SR105 is 3-[2-(3-Acetyl-1-pyrrolidinyl)-1-benzyl-2-oxoethyl]-5-phenyl-2,3-dihydro-4H-1,3-oxazin-4-one
SR107 is 3-[2-(3-Acetyl-1-pyrrolidinyl)-2-oxoethyl]-6-(1-naphthyl)-2,3-dihydro-4H-1,3-oxazin-4-one SR111 is 3-(1-Benzyl-2-oxopropyl)-5-phenyl-2,3-dihydro-4H-1,3-oxazin-4-one
SR206 is 6-Phenacyl-2-phenyl-4H-chromen-4-one
SR328 is 2-(3-Indolyl)-1-(N-6-methyl-1,3-benzothiazol-2-ylcarbamoyl)ethyl phenylmethanecarbamate
SR329 is 2-(3-Indolyl)-1-(N-cycloheptylcarbamoyl)ethyl phenylmethanecarbamate
SR401/SRXXX is 4-Oxo-2-phenyl-4H-chromene-5-carboxylic acid
SR438 (including SR438 Racemic) is 5-[(3-Acetyl-1-pyrrolidinyl)carbonyl]-2-phenyl-4H-chromen-4-one
SR438-R is 5-{[(R)-3-Acetyl-1-pyrrolidinyl]carbonyl}-2-phenyl-4H-chromen-4-one
SR438-S is 5-{[(S)-3-Acetyl-1-pyrrolidinyl]carbonyl}-2-phenyl-4H-chromen-4-one
SR445 is N-[(1,3-Thiazol-4-yl)methyl](S)-3-phenyl-2-[3-(3,4,5-trimethoxyphenyl)propionylamino]propionamide
SR458 is (S)-2-[3-(5-Methoxy-1-indolyl)propionylamino]-3-phenylpropionic acid
SR459 is 3-[2-(3-Acetyl-1-pyrrolidinyl)-2-oxoethyl]-6-phenyl-2,3-dihydro-4H-1,3-oxazin-4-one
SR459 is 3-[2-(3-Acetyl-1-pyrrolidinyl)-2-oxoethyl]-6-phenyl-2,3-dihydro-4H-1,3-oxazin-4-one
SR459-R is 3-{2-[(R)-3-Acetyl-1-pyrrolidinyl]-2-oxoethyl}-6-phenyl-2,3-dihydro-4H-1,3-oxazin-4-one
SR459-S is 3-{2-[(S)-3-Acetyl-1-pyrrolidinyl]-2-oxoethyl}-6-phenyl-2,3-dihydro-4H-1,3-oxazin-4-one
SR462 is (4-Oxo-6-phenyl-3,4-dihydro-2H-1,3-oxazin-3-yl)acetic acid
SR462 is (4-Oxo-6-phenyl-3,4-dihydro-2H-1,3-oxazin-3-yl)acetic acid
SR466 is 3-[2-(2-Acetyl-1-pyrrolidinyl)-2-oxoethyl]-6-phenyl-2,3-dihydro-4H-1,3-oxazin-4-one
SR466-R is 3-{2-[(R)-2-Acetyl-1-pyrrolidinyl]-2-oxoethyl}-6-phenyl-2,3-dihydro-4H-1,3-oxazin-4-one
SR466-S is 3-{2-[(S)-2-Acetyl-1-pyrrolidinyl]-2-oxoethyl}-6-phenyl-2,3-dihydro-4H-1,3-oxazin-4-one
SR490 is 5-[(4-Acetyl-1-piperidyl)carbonyl]-2-phenyl-4H-chromen-4-one
SR495 is 5-[(2-Acetyl-1-piperidyl)carbonyl]-2-phenyl-4H-chromen-4-one
SR496 is 5-[(3-Acetyl-1-piperidyl)carbonyl]-2-phenyl-4H-chromen-4-one
SR498-R is 5-{[(R)-2-Acetyl-1-pyrrolidinyl]carbonyl}-2-phenyl-4H-chromen-4-one
SR498-S is 5-{[(S)-2-Acetyl-1-pyrrolidinyl]carbonyl}-2-phenyl-4H-chromen-4-one
SR582 is 1-Benzyl-2-(2,5-dioxo-1-pyrrolidinyloxy)-2-oxoethyl phenylmethanecarbamate
SR614 is 2-(3-Indolyl)-1-(N-cyclohexylcarbamoyl)ethyl phenylmethanecarbamate
SR710 is 6-{2-[(R)-3-Acetyl-1-pyrrolidinyl]-2-oxoethyl}-2-phenyl-4H-chromen-4-one
SR720 is 3-[2-(3-Acetyl-1-pyrrolidinyl)-1-benzyl-2-oxoethyl]-6-phenyl-2,3-dihydro-4H-1,3-oxazin-4-one
SR720-1 is 3-[2-(3-Acetyl-1-pyrrolidinyl)-1-benzyl-2-hydroxyethyl]-6-phenyl-2,3-dihydro-4H-1,3-oxazin-4-one
SR776 is 2-(3-Indolyl)-1-(N-2-chloroethylcarbamoyl)ethyl phenylmethanecarbamate
SR4384 is 5-(3,3-difluoroazetidine-1-carbonyl)-2-phenyl-4H-chromen-4-one
SR4385 is 5-(3,3-difluoropyrrolidine-1-carbonyl)-2-phenyl-4H-chromen-4-one
SR4386 is 5-(3,3-difluoropiperidine-1-carbonyl)-2-phenyl-4H-chromen-4-one
SR4511 is methyl 1-(2-(4-oxo-6-phenyl-2H-1,3-oxazin-3(4H)-yl)acetyl)pyrrolidine-3-carboxylate
SR4721 is 3-[2-(2-Acetyl-1-azetidinyl)-2-oxoethyl]-6-phenyl-2,3-dihydro-4H-1,3-oxazin-4-one
SR4722 is 3-[2-(3-Acetyl-1-azetidinyl)-2-oxoethyl]-6-phenyl-2,3-dihydro-4H-1,3-oxazin-4-one
SR4722 (Isomer 1) is 3-(2-(3-acetylazetidin-1-yl)-2-oxoethyl)-6-phenyl-2H-1,3-oxazin-4(3H)-one
SR4722 (Isomer 2) is 3-(2-(3-acetylazetidin-1-yl)-2-oxoethyl)-6-phenyl-2H-1,3-oxazin-4(3H)-one
SR5955 is 3-[2-(3,3-Difluoro-1-pyrrolidinyl)-2-oxoethyl]-6-phenyl-2,3-dihydro-4H-1,3-oxazin-4-one
SR5981 is 3-{2-[3-(1-Hydroxyethyl)-1-pyrrolidinyl]-2-oxoethyl}-6-phenyl-2,3-dihydro-4H-1,3-oxazin-4-one
SR7232 is 3-(2-(3,3-difluoropiperidin-1-yl)-2-oxoethyl)-6-phenyl-2H-1,3-oxazin-4(3H)-one
SR7254 is 3-[2-(3,3-Difluoro-1-azetidinyl)-2-oxoethyl]-6-phenyl-2,3-dihydro-4H-1,3-oxazin-4-one
SR7255 is 3-[2-(4,4-Difluoro-1-piperidyl)-2-oxoethyl]-6-phenyl-2,3-dihydro-4H-1,3-oxazin-4-one
SR7280 is 3-(2-Morpholino-2-oxoethyl)-6-phenyl-2,3-dihydro-4H-1,3-oxazin-4-one
SR7301 is 3-(2-(4-methylpiperazin-1-yl)-2-oxoethyl)-6-phenyl-2H-1,3-oxazin-4(3H)-one
SR7302 is 3-(2-(4-acetylpiperazin-1-yl)-2-oxoethyl)-6-phenyl-2H-1,3-oxazin-4(3H)-one SRB3 is N-[2-Hydroxy-3-(4-oxo-6-phenyl-3,4-dihydro-2H-1,3-oxazin-3-yl)-4-phenylbutyl]-N-(3-pyridyl)methylacetamide
SR-J1 is 5-[(3-Acetyl-1-pyrrolidinyl)carbonyl]-4H-chromen-4-one
ZFA is (S)-2-[(S)-2-[Benzyl(oxycarbonylamino)]-3-phenylpropionylamino]propionic acid
ZFP is (S)-1-[(S)-2-[Benzyl(oxycarbonylamino)]-3-phenylpropionyl]-2-pyrrolidinecarboxylic acid

In some examples, the compound activates lysosomal enzymes and/or enhances lysosomal function. In some examples, the compound activates CatB. In some examples, the compound increases the level of mature CatB (CatB-30). In some examples, the compound is free of CatB inhibitory activity. In some examples, the compound increases the ratio of CatB-30 (30 kDa CatB)/CatB-25. In some examples, the compound activates the autophagy-lysosomal pathway. Activation of the autophagy-lysosomal pathway can be determined, for example, by assaying the LC3II/I ratio. In some examples, the compound activates the autophagy-lysosomal pathway better than PADK. In some examples, the compound is free of cellular toxicity and/or off target effects. In some examples, the compound decreases the accumulation of one or more abnormal or potentially harmful proteins (e.g., APP, A3, tau protein, α-synuclein) upon administration of the compound to a subject. In some examples, the compound provides increased degradation of Aβ42, increased clearance of tau, and/or increased clearance of α-synuclein upon administration to a subject. In some examples, the compound provides synaptic protection. In some examples, the compound is SR459, SR466, or SR438. In some examples, the compound is SR459 or SR466. In some examples, the compound is SR466.

In some examples, the presently disclosed subject matter provides a pharmaceutical composition comprising compound of the presently disclosed subject matter (e.g., a compound of Formula (I), (II), and/or (III)) or a pharmaceutically acceptable salt thereof, and a pharmaceutically acceptable carrier. Pharmaceutical compositions comprising the present compounds can be formulated for administration to a subject by any number of routes including, but not limited to, oral, intravenous, intramuscular, microinjection, intra-arterial, intramedullary, intrathecal, intraventricular, transdermal, subcutaneous, intraperitoneal, intranasal, enteral, topical, sublingual, or rectal approaches. In some examples, microinjection is a specialized administration route involving the mechanical introduction of tiny volumes of fluid into cells, tissues, or specific anatomical spaces using microneedles or pipettes.

In some examples, the pharmaceutically acceptable salt is provided by contacting a compound of the presently disclosed subject matter with a suitable acid. Suitable acids include, but are not limited to, inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some examples sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient.

In addition to the active ingredient, a pharmaceutical composition of the presently disclosed subject matter can further comprise one or more additional pharmaceutically active agents.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Suitable coloring agents include red, black, and yellow iron oxides and FD&C dyes such as FD&C Blue No. 2, FD&C Red No. 40, and the like. Suitable flavoring agents include mint, raspberry, licorice, orange, lemon, grapefruit, caramel, vanilla, cherry grape flavors, combinations thereof, and the like. Suitable pH modifiers include citric acid, tartaric acid, phosphoric acid, hydrochloric acid, maleic acid, sodium hydroxide, and the like. Suitable sweeteners include aspartame, acesulfame K, thaumatic, and the like. Suitable taste-masking agents include sodium bicarbonate, ion-exchange resins, cyclodextrin inclusion compounds, adsorbates, and the like.

Typically, dosages of the compound of the presently disclosed subject matter which can be administered to an animal, in some examples a human, range in amount from 1 g to about 100 g per kilogram of body weight of the animal. While the precise dosage administered will vary depending upon any number of factors, including but not limited to, the type of animal and type of disease state being treated, the age of the animal, the size of the animal, the gender of the animal, the condition of the animal, and the route of administration. In some examples, the dosage of the compound will vary from about 1 mg to about 10 g per kilogram of body weight of the animal. In another aspect, the dosage will vary from about 10 mg to about 1 g per kilogram of body weight of the animal.

The compositions can be administered to an animal as frequently as several times daily, or it can be administered less frequently, such as once a day, once a week, once every two weeks, once a month, or even less frequently, such as once every several months or even once a year or less.

### IV. Methods for Positive Modulation of the Autophagy-Lysosomal Pathway

In some examples, the presently disclosed subject matter provides a method of positively modulating cathepsin β in a sample comprising cathepsin β, the method comprising contacting the sample with a compound of the presently disclosed subject matter (e.g., a compound of Formula (I) or (II)) or a pharmaceutical composition thereof. In some examples, the presently disclosed subject matter provides the use of a compound of the presently disclosed subject matter (e.g., a compound of Formula (I) or (II)) or a pharmaceutical composition thereof, for positively modulating cathepsin β in a sample comprising cathepsin β. In some examples, the compound is selected from the group consisting of SR466, SR71, SR70, SR72, SR401, SR459, SR466, SR462, SR103, SR103B, SR104, SR105, SR107, SR111, SR720, SR720-1, SRB3, SR70, SR71, SR72, SR4721, SR4722, SR7254, SR7280, SR7255, SR5955, SR5981, SR-J1, SR438, SR498, SR490, SR495, SR496, SR206, SR4386, SR4385, SR4384, SR4511, SR7301, SR7302, SR7232, and SR710, optionally SR459, SR466, SR462, and SR438. In some examples, the compound is SR438, SR498, SR459 or SR466, optionally wherein the compound is SR438-S, SR438-R, SR498-S, SR498-R, SR459-S, SR459-R, SR466-S, or SR466-R. In some examples, the compound is SR4722, isomer 1 and/or SR4722, isomer 2.

In some examples, the compound is one of:

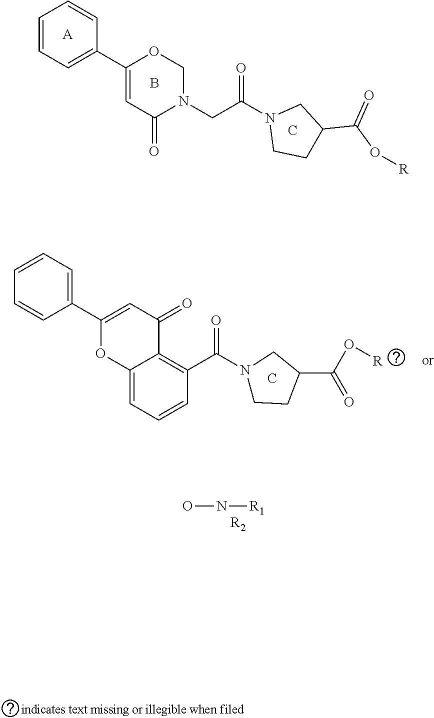

Looking at the above-presented examples, with representative, non-limiting designated A B and C ring moieties, the C-ring is a 4-, 5- (as pictured), 6-, 7-, or 8-member ring, which can be substituted or unsubstituted, e.g. a substituted or unsubstituted alkylene or aralkylene group. “Substituted” here is meant to encompass all the definitions presented herein for the term “substituted”, including but not limited to “alkyl group substituent”, “aryl group substituent”, “substituted alkyl”, and “substituted aryl”, as set forth elsewhere herein. Thus, there can be optionally inserted along the 4-, 5-, 6-, 7-, or 8-membered ring one or more oxygen, sulfur or substituted or unsubstituted nitrogen atoms. In some examples, the side group coming off the C-ring of both structures can include a methyl ester change in addition to methyl ketone chemistry also described herein. The compounds can include branching off with an amide functional group. In the examples presented above, R, R₁, and R₂ can include but are not limited to H, acyl, alkyl, substituted alkyl, aralkyl, alkoxy, substituted aralkyl, aryl, or substituted aryl, or wherein R₁, and R₂ together form a substituted or unsubstituted alkylene or aralkylene group. Guidance for preparing these compounds can be found elsewhere herein, including the Examples presented below.

The sample can be a living organism (e.g., a subject) or a sample from a subject. In some examples, the sample is a biological sample from a subject, including, but not limited to, normal tissue samples, diseased tissue samples, biopsies, blood, saliva, feces, semen, tears, and urine. A sample can also be any other source of material from a subject which contains proteins, cells, tissues, or a fluid of interest. A sample can also be obtained from cell or tissue culture. In some examples, the contacting can result in increased CatB-30. In some examples, the contacting provides increased degradation of Aβ42, increased clearance of tau, and/or increased clearance of α-synuclein.

In some examples, the presently disclosed subject matter provides a method of treating or preventing a neurodegenerative disease, disorder or condition in a subject (e.g., in a mammalian subject) in need of treatment or at risk thereof, wherein the method comprises administering to the subject an effective amount of a compound of the presently disclosed subject matter (e.g., a compound of Formula (I), (II), and/or (III)). In some examples, the compound is selected from the group consisting of SR466, SR71, SR70, SR72, SR401, SR459, SR466, SR462, SR103, SR103B, SR104, SR105, SR107, SR111, SR720, SR720-1, SRB3, SR70, SR71, SR72, SR4721, SR4722, SR7254, SR7280, SR7255, SR5955, SR5981, SR-J1, SR438, SR498, SR490, SR495, SR496, SR206, SR4386, SR4385, SR4384, SR4511, SR7301, SR7302, SR7232, and SR710, optionally SR459, SR466, SR462, and SR438. In some examples, the compound is SR438, SR498, SR459 or SR466, optionally wherein the compound is SR438-S, SR438-R, SR498-S, SR498-R, SR459-S, SR459-R, SR466-S, or SR466-R. In some examples, the compound is SR4722, isomer 1 and/or SR4722, isomer 2.

In some examples, the compound is one of:

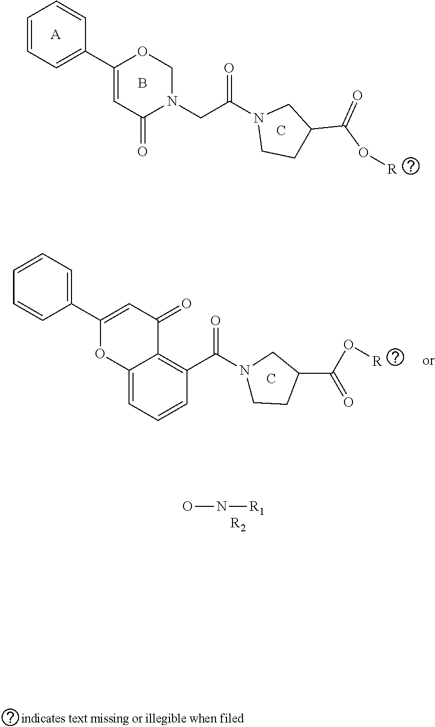

Looking at the above-presented examples, with representative, non-limiting designated A B and C ring moieties, the C-ring is a 4-, 5- (as pictured), 6-, 7-, or 8-member ring, which can be substituted or unsubstituted, e.g. a substituted or unsubstituted alkylene or aralkylene group. “Substituted” here is meant to encompass all the definitions presented herein for the term “substituted”, including but not limited to “alkyl group substituent”, “aryl group substituent”, “substituted alkyl”, and “substituted aryl”, as set forth elsewhere herein. Thus, there can be optionally inserted along the 4-, 5-, 6-, 7-, or 8-membered ring one or more oxygen, sulfur or substituted or unsubstituted nitrogen atoms. In some examples, the side group coming off the C-ring of both structures can include a methyl ester change in addition to methyl ketone chemistry also described herein. The compounds can include branching off with an amide functional group. In the examples presented above, R, R₁, and R₂ can include but are not limited to H, acyl, alkyl, substituted alkyl, aralkyl, alkoxy, substituted aralkyl, aryl, or substituted aryl, or wherein R₁, and R₂ together form a substituted or unsubstituted alkylene or aralkylene group. Guidance for preparing these compounds can be found elsewhere herein, including the Examples presented below.

In some examples, the neurodegenerative disease, disorder or condition is a protein accumulation disorder. In some examples, neurodegenerative disease, disorder, or condition is selected from Alzheimer's disease, Parkinson's disease, Huntington's disease, amyotrophic lateral sclerosis, mild cognitive impairment (MCI) or traumatic brain injury (TBI).

In some examples, the subject is a human subject. In some examples, the subject is a human subject who has been diagnosed with a neurodegenerative disease, disorder or condition (e.g., a protein accumulation disorder) using one or more of method known in the art. Such methods include, but are not limited to, a cognitive test, a family history assessment, a blood test, a cerebrospinal fluid (CSF) test, a neuronal connectivity scan, a white matter integrity analysis, a magnetic resonance imaging (MRI) test of gray matter, an assessment of the microstructural integrity of axons and their surrounding myelin, a glucose metabolism assessment, a cortical thickness analysis, a multimodality biomarker assessment, genomic analysis, a sarcasm detection assessment, or any combination thereof. In some examples, the subject is a subject at risk for developing a neurodegenerative disease, disorder or condition, e.g., based on an age, genetics/family history, a history of socio-economic stressors, and/or past exposure to military blasts or toxins.

In some examples, administering can result in one, two, three, four, five, or more, or all of the symptoms of the neurodegenerative disease, disorder or condition to be reduced, reversed, or prevented. In some examples, the progression of one, two, three, four, five, or more, or all of the symptoms of the neurodegenerative disease, disorder or condition are slowed or reversed.

In some examples, the presently disclosed subject matter provides a compound of the presently disclosed subject matter (e.g., a compound of Formula (I) or (II)), or a pharmaceutically acceptable salt and/or pharmaceutical composition thereof, for use in treating or preventing a neurodegenerative disease, disorder or condition in a subject (e.g., in a mammalian subject) in need of treatment or at risk thereof, wherein the method comprises administering to the subject an effective amount of the compound, salt and/or composition. In some examples, the compound is selected from the group consisting of SR466, SR71, SR70, SR72, SR401, SR459, SR466, SR462, SR103, SR103B, SR104, SR105, SR107, SR111, SR720, SR720-1, SRB3, SR70, SR71, SR72, SR4721, SR4722, SR7254, SR7280, SR7255, SR5955, SR5981, SR-J1, SR438, SR498, SR490, SR495, SR496, SR206, SR4386, SR4385, SR4384, SR4511, SR7301, SR7302, SR7232, and SR710, optionally SR459, SR466, SR462, and SR438. In some examples, the compound is SR438, SR498, SR459 or SR466, optionally wherein the compound is SR438-S, SR438-R, SR498-S, SR498-R, SR459-S, SR459-R, SR466-S, or SR466-R. In some examples, the compound is SR4722, isomer 1 and/or SR4722, isomer 2.

In some examples, the compound is one of:

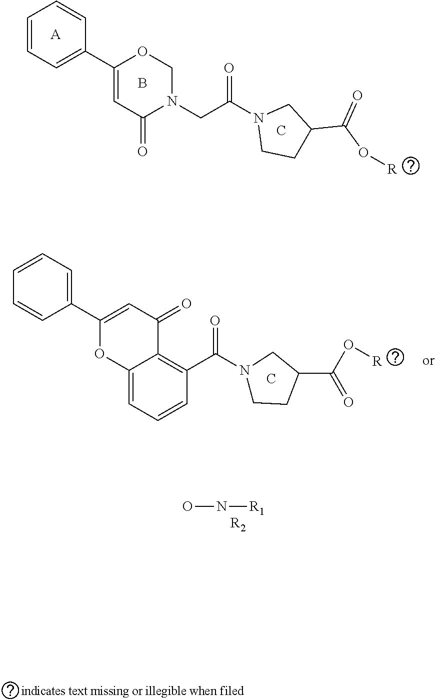

Looking at the above-presented examples, with representative, non-limiting designated A B and C ring moieties, the C-ring is a 4-, 5- (as pictured), 6-, 7-, or 8-member ring, which can be substituted or unsubstituted, e.g. a substituted or unsubstituted alkylene or aralkylene group. “Substituted” here is meant to encompass all the definitions presented herein for the term “substituted”, including but not limited to “alkyl group substituent”, “aryl group substituent”, “substituted alkyl”, and “substituted aryl”, as set forth elsewhere herein. In some examples, the side group coming off the C-ring of both structures can include a methyl ester change in addition to methyl ketone chemistry also described herein. Thus, there can be optionally inserted along the 4-, 5-, 6-, 7-, or 8-membered ring one or more oxygen, sulfur or substituted or unsubstituted nitrogen atoms. The compounds can include branching off with an amide functional group. In the examples presented above, R, R₁, and R₂ can include but are not limited to H, acyl, alkyl, substituted alkyl, aralkyl, alkoxy, substituted aralkyl, aryl, or substituted aryl, or wherein R₁, and R₂ together form a substituted or unsubstituted alkylene or aralkylene group. Guidance for preparing these compounds can be found elsewhere herein, including the Examples presented below.

In some examples, the neurodegenerative disease, disorder or condition is a protein accumulation disorder. In some examples, neurodegenerative disease, disorder, or condition is selected from Alzheimer's disease, Parkinson's disease, Huntington's disease, amyotrophic lateral sclerosis, mild cognitive impairment (MCI) or traumatic brain injury (TBI).

The activity of the presently disclosed compounds can be tested in hippocampal slice cultures. See e.g., Farizatto et al. 2017 PLoS ONE 12: e0182895. Activity can also be assessed using various animal models of particular neurodegenerative diseases, disorders, or conditions known in the art. See e.g., Pepeu 2004, Dialogues in Clinical Neuroscience, 6(4):369-377.

While it is not desired to be bound by any particular theory of operation, in some examples, compounds of the presently disclosed subject matter target at least one lysosomal enzyme, promote cathepsin maturation that may involve lysosomal acidification, restore trafficking of cargoes to active lysosomes, increase breakdown of lysosomal cargo (e.g. key lysosomal cargos in Alzheimer's disease including amyloid-β peptides), and/or improve lysosome resilience to age-related compromise and pathogenic damage. In some examples, specific enhancing effects on cathepsin B occur for the intracellular maturation of the enzyme into the active single chain form (30-33 kDa) as well as for the secondary processing into the double chain form of cathepsin B detected by measuring the 25-28-kDa heavy chain of this form. Cathepsin B has been previously shown to proteolytically cleave amyloid-β peptides and to facilitate their clearance from brain tissue.

### EXAMPLES

The following Examples have been included to provide guidance to one of ordinary skill in the art for practicing representative examples of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Synthesis of Exemplary Compounds

Exemplary compounds of Formula (I), i.e., SR 462, SR466, and SR459, were prepared as shown below in Scheme 1.

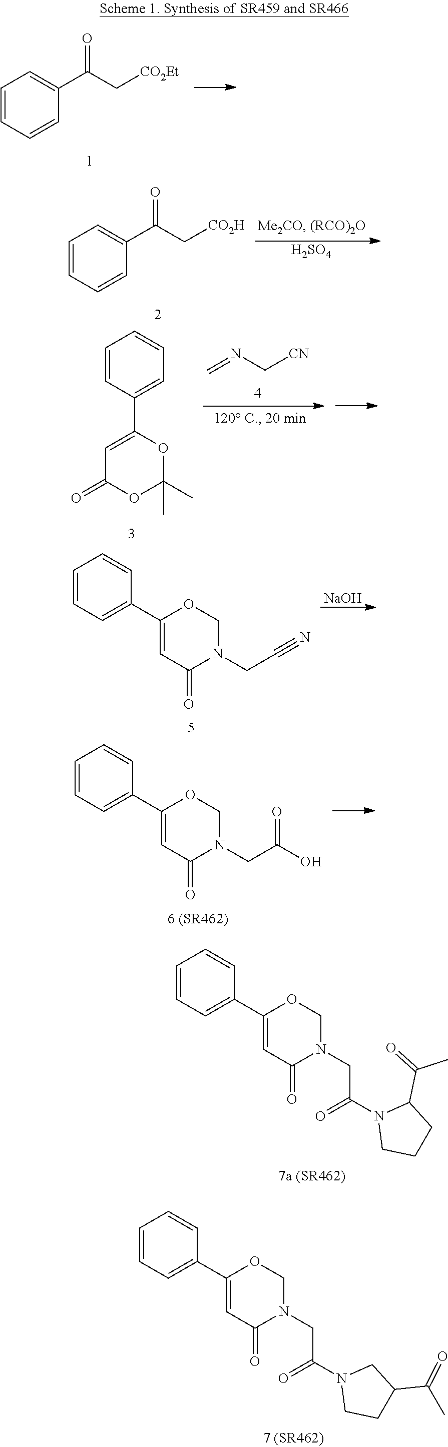

### Synthesis of Compound 2

As shown in Scheme 1, to ethyl benzoylacetate 1 (32.7 g, 0.17 mol) was added a 1 N solution of NaOH in water (170 mL). The reaction mixture was stirred at ambient temperature for 16 h, then extracted with ether (200 mL×3). To the water layer was added HCl conc. up to pH 1, the precipitate of compound 2 was filtered, washed with water (200 mL) and dried yielding acid 2 (16 g, 49%), as a white solid.

### Synthesis of Compound 3

Continuing with Scheme 1, above, to a stirred mixture of acid 2 (1.43 g, 8.7 mmol), acetone (1 g, 17.4 mmol) and acetic anhydride (1.77 g, 17.4 mmol) was added H₂SO₄ (82 mg, 0.87 mmol). The reaction mixture was stirred at 0° C. for 3 h and then was maintained at 2° C. for 16 h. Then the solution was poured into a 10% Na₂CO₃ at 0° C. and stirred for 30 min. Product (compound 3) was filtered, washed with water (20 mL) and dried yielding compound 3 (0.96 g, 48%),

### Synthesis of Compound 5

Continuing with Scheme 1, compound 3 (0.96 g, 4.7 mmol) and methyleneaminoacetonitrile 4 (0.32 g, 4.7 mmol) were stirred and heated at 120° C. for 20 min, then the reaction mixture was cooled, dissolved in dichloromethane and separated by flash chromatography (EtOAc/hexane, 1:3) yielding compound 5 (SR463) (47 g, 47%), as a white solid.

### Synthesis of Compound 6

Continuing with Scheme 1, to a solution of compound 5 (0.47 g, 2.2 mmol) in ethanol (20 mL) were added water (2 mL) and NaOH (0.13 g. 3.3 mmol), then the solution was stirred under reflux for 2 h, cooled, evaporated under reduce pressure, to the residue water (5 mL) was added and added HCl conc. until pH 2. Precipitate of compound 6 was filtered, washed with water (2 mL) and dried yielding acid 6 (SR462) (0.38 g, 72%), as a white solid.

### Synthesis of Compounds 7a

Continuing with Scheme 1, to a solution of compound 6 (90 mg, 0.39 mmol) in DCM (2 mL) were added TEA (94 mg, 0.92 mmol) and TBTU (148 mg, 0.46 mmol), and then the amine hydrochloride (57 mg, 0.39 mmol). The reaction mixture was stirred at 25° C. for 6 h, then DCM (7 mL) was added, the solution was washed with water (1 mL). Product was isolated via a column chromatography separation (dichloromethane/EtOAc) and followed by an additional purification by a preparative HPLC yielding compound 7a (SR466) (25 mg, 21%).

### Synthesis of Compound 7

To a solution of compound 6 (90 mg, 0.39 mmol) in DCM (2 mL) were added TEA (94 mg, 0.92 mmol) and TBTU (148 mg, 0.46 mmol), and then the amine hydrochloride (57 mg, 0.39 mmol). The reaction mixture stirred at 25° C. for 6 h, then DCM (7 mL) was added, the solution was washed with water (1 mL). Product was isolated via a column chromatography separation (dichloromethane/EtOAc) and followed by an additional purification by a preparative HPLC yielding compound 7 (SR459) (35 mg, 29%).

Additional compounds of Formula (I) can be prepared by using similar synthesis routes, e.g., using other beta-keto acetates or beta-keto acetates in place of compound 1 or 2 or by using a different amine to form the amide in the last step of the synthesis of Scheme 1.

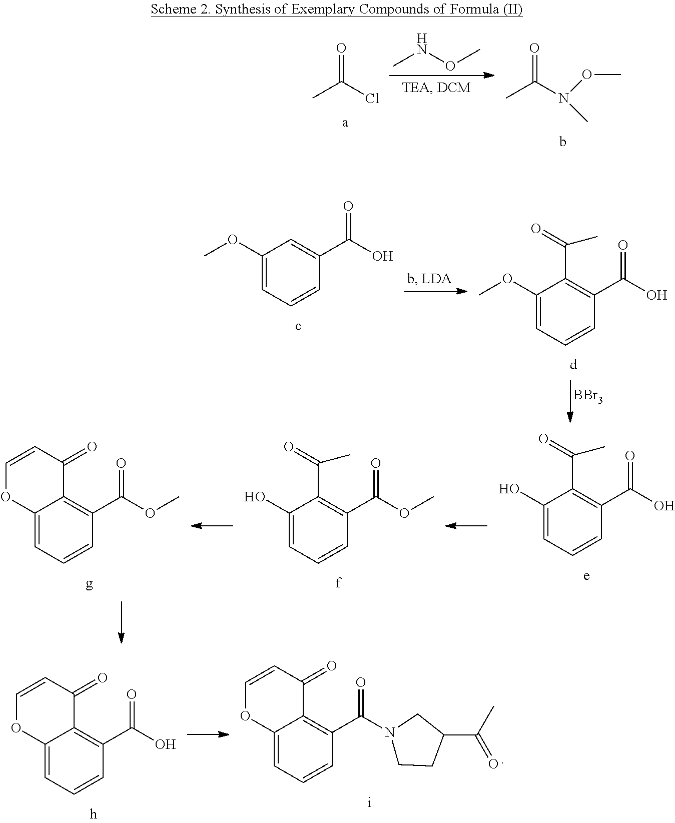

Compounds of Formula (II) can be prepared via routes such as shown in Scheme 2, above. More particularly, as shown in Scheme 2, to provide compound i (i.e., SR-J1) acetamide b can be prepared by reacting acid chloride a (i.e., acetyl chloride) with N,O-dimethylhydroxylamine. Acetamide b can be reacted with methoxy-substituted aryl compound c (i.e., 3-methoxybenzoic acid) to form aryl ketone d in the presence of an organolithium reagent (e.g., lithium diisopropylamide (LDA)). Then, the methoxy group of ketone d is converted to a phenol by reacting d with boron tribromide (BBr₃), forming α-hydroxyketone e. The carboxylic acid group of e can be protected by converting it to a methyl ester, forming compound f. Compound f can be converted to compound g via an O-acylation and aldol condensation reaction (referred to as a Kostanecki-Robinson reaction) with an acid anhydride (acetic anhydride) in the presence of a sodium or potassium salt of the corresponding acid (e.g., sodium acetate). The ester group in compound g is then hydrolyzed to provide carboxylic acid h which can be converted to amide i via reaction with 3-acetylpyrrole. Other compounds of Formula (III) can be prepared by using other methoxy-substituted aryl compounds, using other acid anhydrides (e.g., benzoic anhydride) in place of the acetic anhydride, or by using different nitrogen-containing heterocycles or amines in place of the 3-acytl pyrrole in the final step. Other routes to compounds of Formula (II) can also be used, e.g., routes involving a Perkin reaction or a Perchmann condensation.

Compound SR438 is example compound of Formula (II) and can be prepared using the following non-limiting example scheme:

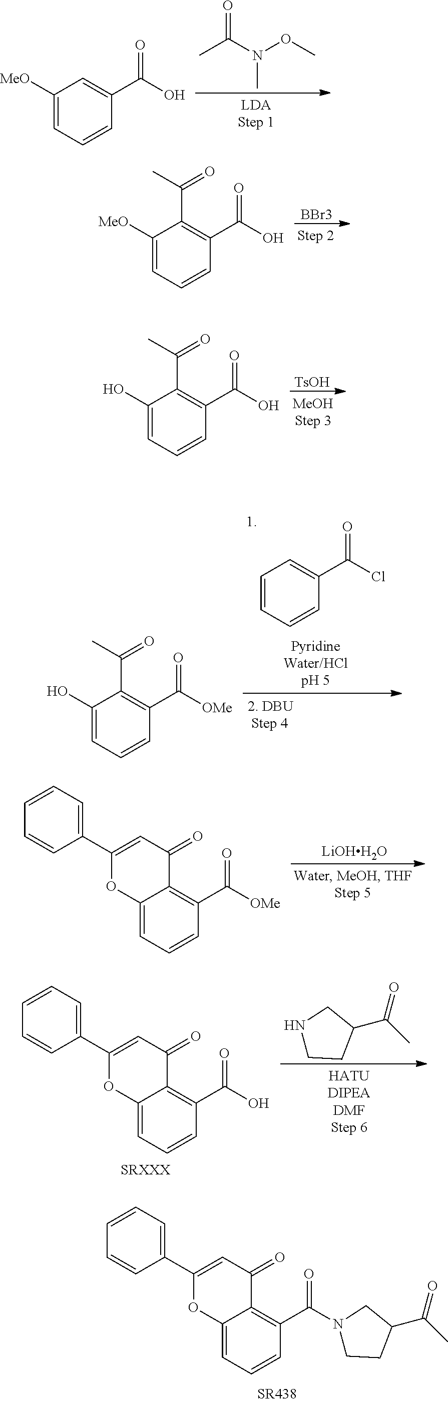

The compound number for SRXXX above is SR401.

### Example 2

### Biological Activity Assays

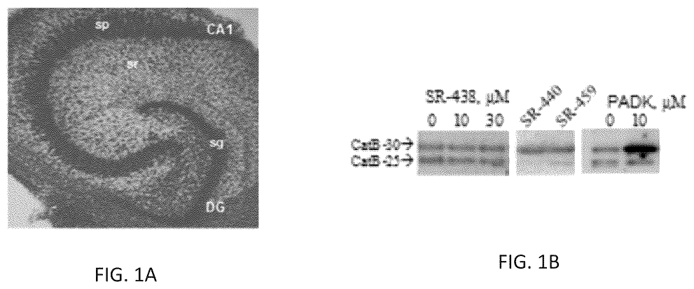

Rat hippocampal explants were used in non-limiting examples for screening compounds. The organotypic hippocampal transverse slice cultures were maintained on Biopore inserts for 3-6 weeks, exhibiting native organization of Nissl-stained neuronal fields. See FIG. 1A. Various compounds of the presently disclosed subject matter, as well as previously described compounds, including ZFA and ZFP (see U.S. Pat. Nos. 8,163,953, and 10,702,571, the disclosures of which are incorporated herein by reference in their entirety), were applied to organotypic hippocampal slice cultures for 48 hours to determine their effect on the active form ofcathepsin B (CatB-30) by immunoblotting. As compared to vehicle-treated slice samples, immunoreactivity levels for the 30-kDa isoform were increased by 100-300% (+++), 50-99% (++), 25-49% (+), by a small, inconsistent amount (+/−), or exhibited no effect by the treatment (NE). See Table 1, below. FIG. 1B also shows immunoreactivity levels for CatB-30 and CatB-25 for samples treated with 10 μM PADK, SR440, SR459, or 10-30 μM SR438.

TABLE 1
Hippocampal Slice Immunoreactivity Assay

| | | | | CatB-30 |
|---|---|---|---|---|
| | | Molecular | | enhancing |
| Name | Structure | Formula | MW | effect |
| ZFA | 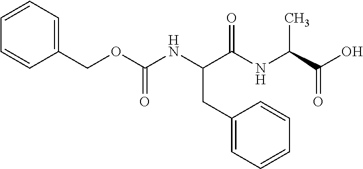 [SMILES: C[C@H](NC(=O)C(Cc1ccccc1)NC(=O)OCc1ccccc1)C(=O)O] | C₂₀H₂₂N₂O₅ | 370.4 | NE |
| ZFP | 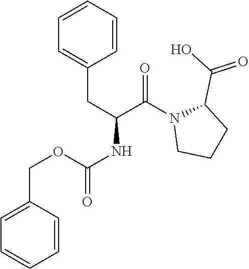 [SMILES: O=C(N[C@@H](Cc1ccccc1)C(=O)N1CCC[C@H]1C(=O)O)OCc1ccccc1] | C₂₂H₂₄N₂O₅ | 396.44 | ++ |
| SR440 | 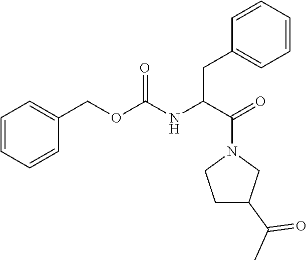 [SMILES: CC(=O)C1CCN(C(=O)C(Cc2ccccc2)NC(=O)OCc2ccccc2)C1] | C₂₃H₂₆N₂O₄ | 394.47 | ++ |
| SR441 | 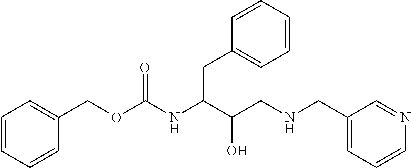 [SMILES: O=C(NC(Cc1ccccc1)C(O)CNCc1cccnc1)OCc1ccccc1] | C₂₄H₂₇N₃O₃ | 405.5 | +++ |
| SR438 | 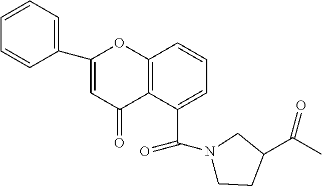 [SMILES: CC(=O)C1CCN(C(=O)c2cccc3oc(-c4ccccc4)cc(=O)c23)C1] | C₂₂H₁₉NO₄ | 361.40 | + |
| SR459 | 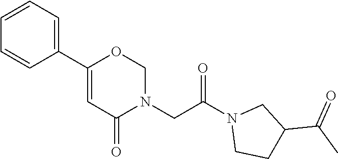 [SMILES: CC(=O)C1CCN(C(=O)CN2COC(c3ccccc3)=CC2=O)C1] | C₁₈H₂₀N₂O₄ | 328.3 | +++ |
| SR466 | 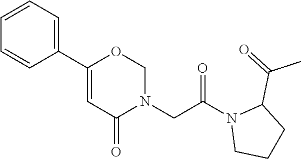 [SMILES: CC(=O)C1CCCN1C(=O)CN1COC(c2ccccc2)=CC1=O] | C₁₈H₂₀N₂O₄ | 328.3 | +++ |
| SR614 | 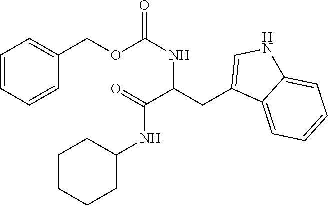 [SMILES: O=C(NC(Cc1c[nH]c2ccccc12)C(=O)NC1CCCCC1)OCc1ccccc1] | | 419.5 | + |
| SR776 | 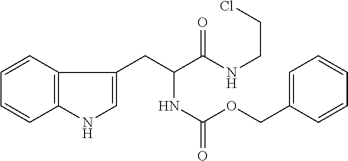 [SMILES: O=C(NC(Cc1c[nH]c2ccccc12)C(=O)NCCCl)OCc1ccccc1] | | 399.88 | + |
| SR582 | 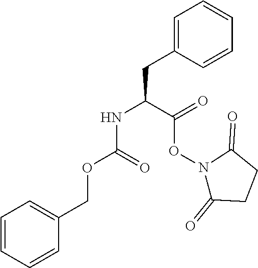 [SMILES: O=C(N[C@@H](Cc1ccccc1)C(=O)ON1C(=O)CCC1=O)OCc1ccccc1] | | 396.4 | +/− |
| SR458 | 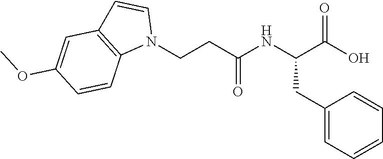 [SMILES: COc1ccc2c(ccn2CCC(=O)N[C@@H](Cc2ccccc2)C(=O)O)c1] | | 366.4 | +/− |
| SR328 | 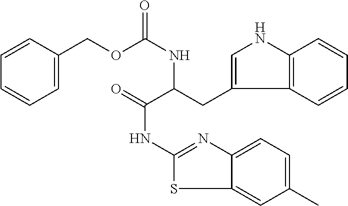 [SMILES: Cc1ccc2nc(NC(=O)C(Cc3c[nH]c4ccccc34)NC(=O)OCc3ccccc3)sc2c1] | | 484.58 | +/− |
| SR445 | 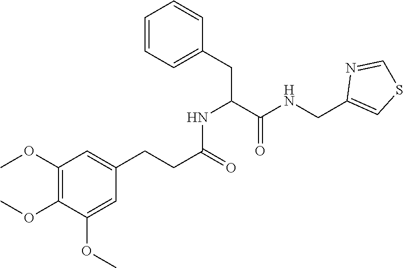 [SMILES: COc1cc(CCC(=O)NC(Cc2ccccc2)C(=O)NCc2cscn2)cc(OC)c1OC] | | 483.59 | +/− |
| SR329 | 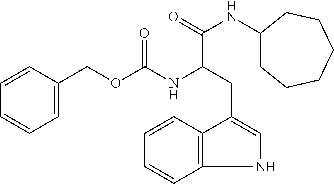 [SMILES: O=C(NC(Cc1c[nH]c2ccccc12)C(=O)NC1CCCCCC1)OCc1ccccc1] | | 433.5 | +/− |
| SR462 | 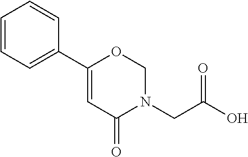 [SMILES: O=C(O)CN1COC(c2ccccc2)=CC1=O] | | | NE |

Organotypic hippocampal cultures were treated with different concentrations of SR459 for 48 hours. Prepared tissue samples from the harvested slice cultures were subjected to the CalBiochem cathepsin B assay kit that utilizes the fluorogenic substrate Z-Arg-Arg AMC. The results, which show the dose-dependent increase in CatB activity by SR459.

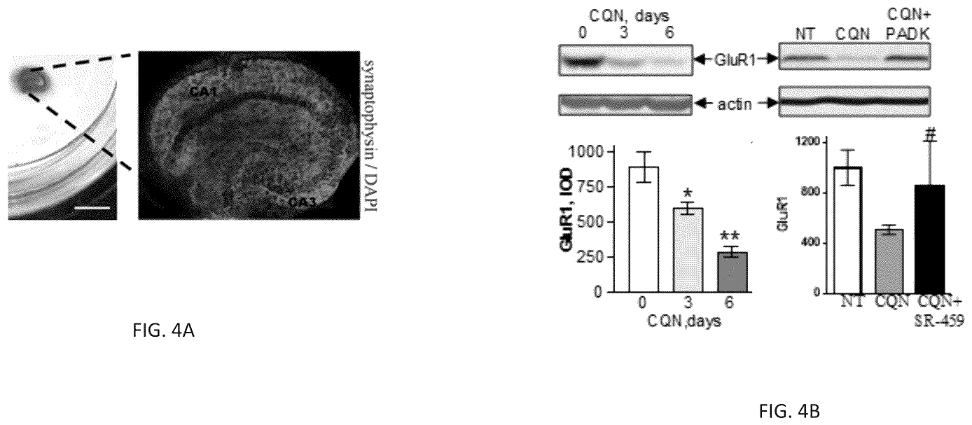

Different classes of CatB-targeting compounds were tested for direct action on CatB activity in lysed cells. As shown in FIG. 3, compared to the not treated control, 45 M SR459 increased CatB activity by 49.9%. In contrast, 45 μM PADK and 15 μM CA074 reduced activity by 85.8% and 87.3%, respectively.

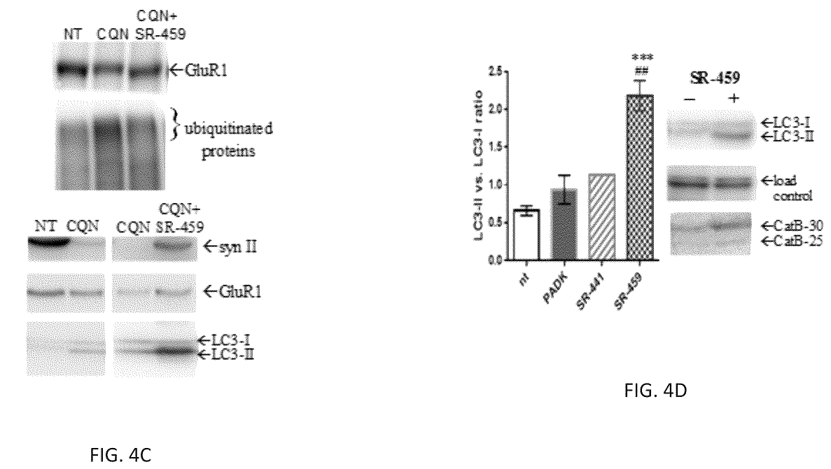

The protective effects of different compounds were assessed in hippocampal explants exhibiting synaptopathy induced by the chloroquine (CQN) to experimentally model age-related lysosomal dysfunction. See FIG. 4A-4D. Treatment with SR459 protected from chloroquine-induced reduction of the postsynaptic marker GluR1. See FIG. 4B. The postsynaptic protection by SR459 included protected levels of the presynaptic marker synapsin II (syn II) and associated reduction in chloroquine-induced protein ubiquitination. See FIG. 4C. SR459 was also associated with an increase in the LC3-II/LC3-I indicator of autophagy activation. See FIG. 4D.

### Example 3

### Studies in MCI Model

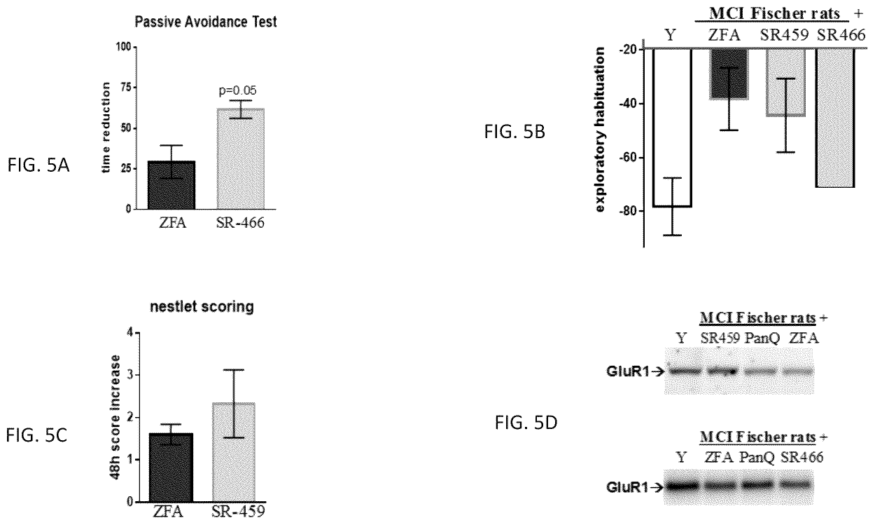

FIGS. 5A-5D show the results of studies performed with SR459 and SR466 in a rat model of MCI. Female Fischer rats of 12-13 months of age received 6-7 daily injections for 20 mg·kg/day of inactive ZFA compound, 15 mg/kg/day of SR466 or 12 mg/kg/day of SR459. Select groups of young (3 months old) and treated Fischer rats were tested with behavioral paradigms before rapid dissection. In those tested for passive avoidance, rats treated with SR466 exhibited improved learning compared to ZFA-treated MCE rats, as indicated by enhanced avoidance of the shock area. See FIG. 5A. Exploratory habituation was assessed in an open field for monitoring movement. The young group exhibited the largest percentage reduction in exploring the same environment again on day two as compared to day one, whereas the MCI group given the control compound had the smallest reduction. See FIG. 5B. Those MCI rats injected with SR compounds exhibited varying levels of improved exploratory habituation behavior. Assessment of nest building/nestlet shredding was conducted as a deficit of this behavioral phenotype has been reported as a starting feature of neurological disorders. The MCI rats treated with SR-459 had evident improvement in nestlet scores compared to ZFA-treated rats. See FIG. 5C.

Following dissection, equal aliquots of hippocampal homogenates were prepared from the different rat groups, including MCI rats treated with the natural product PanQ. These aliquots were subjected to GluR1 immunostaining on separate immunoblots. See FIG. 5D.

### Example 4

### Discussion of Examples 1-3

Previous studies with CatB enhancing compound PADK showed that upregulation of the active form of cathepsin B is associated with reduced levels of Aβ peptide, carboxyterminal fragments of APP (CTFs), TDP-43 (a disease protein associated with ubiquitin-positive, tau- and α-synuclein-negative FTD and in ALS), phosphorylated tau (pTau), and α-synuclein (α-syn). See Butler et al. (2011) PLoS ONE 6:e20501; Farizatto et al. (2017) PLoS One 12:e0182895; and Hwang et al. (2019) International J. Mol. Sci. 20:4432. These studies suggest that enhancing a protease of the autophagy-lysosomal pathway can slow or reverse protein accumulation pathology, inspiring a search for additional cathepsin B enhancing molecules (“CatB enhancer”), particularly those that are free of CatB inhibitory activity, to offset multi-proteinopathy associated with various diseases and conditions, such as AD, PD, and MCI.

Table 2, below, shows that an exemplary compound of Formula (I) described herein, SR-459 combines several beneficial biological properties.

TABLE 2
Compound Characteristics

| | | | | | | Free of |
|---|---|---|---|---|---|---|
| | | | | | | cellular |
| | | | Free of | | | toxicity |
| | | | CatB | Increases | | and 44 |
| | Increases | Increases | inhibitory | LC3- | Synaptic | receptor |
| Compound | CatB-30 | CatB30/25 | activity | II/LC3-I | protective | flags |
| SR459 | Yes | Yes | Yes | Yes | Yes | Yes |
| SR459-R | Yes | Yes | n.d. | n.d. | n.d. | n.d. |
| SR441 | Yes | Yes | n.d. | No | No | n.d. |
| PADK | Yes | Yes | No | Yes | Yes | No |
| BW1002* | Yes | No | No | n.d. | Yes | n.d. |

*See U.S. Pat. No. 8,163,953

Table 2 shows that SR-459 combines several positive characteristics, including:
increasing the 30-kDa active form of cathepsin B in brain tissue;
increasing the CatB-30/CatB-25 ratio (indication of enzyme maturation);
being free of cathepsin B inhibitory activity (inducing an increase at 45 M);
increasing the LC3-II/LC3-I ratio, an indication of autophagy activation;
eliciting synaptic protection in a model of age-related lysosomal and synaptic compromise; and
freedom from cellular toxicity and off-target receptor effects (based on screening in 44 receptor binding, enzyme, and uptake assays).

### Example 5

### Synthesis of SR462

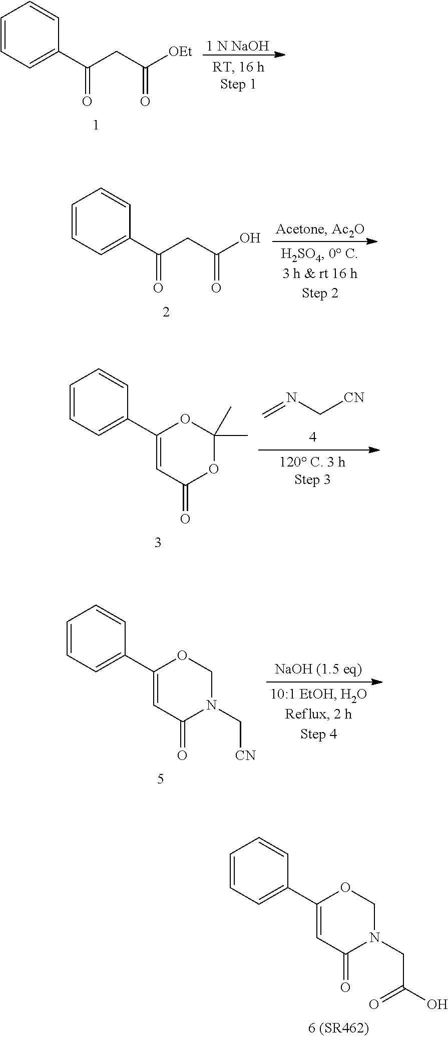

### Step-1: Synthesis of 3-oxo-3-phenylpropanoic acid (2)

To a stirred solution of ethyl benzoylacetate (25.0 g, 140 mol, 1.0 eq.) was added a 1 N solution of NaOH (154 mL, 154 mmol, 1.1 eq.) in water. The reaction mixture was stirred at ambient temperature for 16 h. The solution was washed with ether (3×150 mL). To the water layer was added conc. HCl until pH 1. The resulting precipitate was filtered, the solid residue was washed with water (200 mL), and dried under reduced pressure yielding 3-oxo-3-phenylpropanoic acid (2) (13 g, 56% yield), as a white solid.

### Step-2: Synthesis of 2,2-dimethyl-6-phenyl-4H-1,3-dioxin-4-one (3)

To a round bottom flask was added 3-oxo-3-phenylpropanoic acid (2) (13.0 g, 79.2 mmol), acetone (11.7 mL, 158 mmol), acetic anhydride (1.77 g, 17.3 mmol) and H₂SO₄ (0.43 mL, 7.92 mmol) at ° C. The reaction mixture was stirred at 0° C. for 3 h, then was allowed to stir at room temperature for 16 h. Then the solution was poured into a 10% Na₂CO₃ at 0° C. and was stirred for 30 min. The resulting solid was filtered, the solid residue was washed with water (20 mL), and was dried, yielding compound 2,2-dimethyl-6-phenyl-4H-1,3-dioxin-4-one (3) (13.0 g, 80% yield) as a light-yellow solid.

### Step-3: Synthesis of 2-(4-oxo-6-phenyl-2H-1,3-oxazin-3(4H)-yl)acetonitrile (5)

2,2-dimethyl-6-phenyl-4H-1,3-dioxin-4-one (3) (12.5 g, 61.2 mmol) and methylene aminoacetonitrile (4.16 g, 61.2 mmol) were stirred and heated at 120° C. for 3 h, then the reaction mixture was cooled, dissolved in dichloromethane and purified via silica gel flash chromatography (25% EtOAc-hexanes) yielding 2-(4-oxo-6-phenyl-2H-1,3-oxazin-3(4H)-yl)acetonitrile (5) (3.00 g, 22% yield) as a white solid.

### Step-4: Synthesis of 2-(4-oxo-6-phenyl-2H-1,3-oxazin-3(4H)-yl)acetic acid (SR462)

To a stirring solution of 2-(4-oxo-6-phenyl-2H-1,3-oxazin-3(4H)-yl)acetonitrile (4) (3.00 g, 14.0 mmol) in ethanol (30 mL) was added water (6 mL) and NaOH (0.84 g. 21.0 mmol), then the solution was stirred under reflux for 2 h. The mixture was cooled to room temperature, was concentrated under reduce pressure. The resulting residue was treated with conc. HCl until pH 2. The resulting precipitate was filtered, washed with water (2 mL) and dried yielding 3-oxo-3-phenylpropanoic acid (SR462) (1.50 g, 46% yield), as a white solid. ¹HNMR (600 MHz, CDCl₃) δ 7.79-7.78 (d, 2H), 7.53-7.46 (m, 3H), 6.07 (s, 1H), 5.41 (s, 2H), 5.14 (s, 2H). MS (M+H)⁺ 234.2 (m/z). HPLC: 97.1% AUC.

### Example 6

### Synthesis of SR71

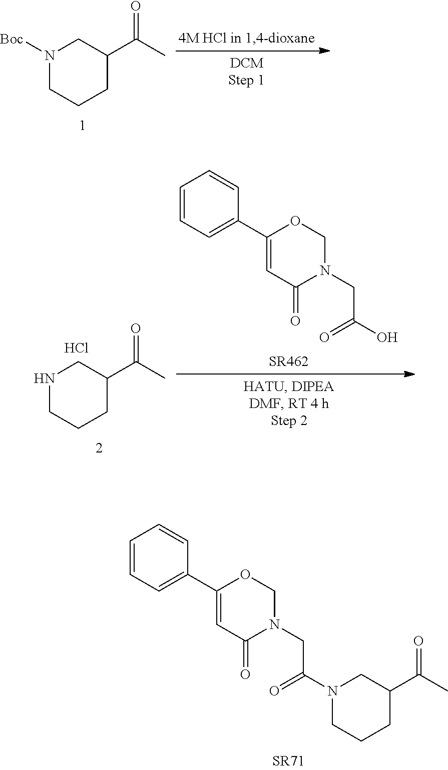

### Step 1: Preparation of 1-(piperidin-3-yl)ethanone hydrochloride (2)

To a stirring solution of tert-butyl 3-acetylpiperidine-1-carboxylate (1) (500 mg, 2.20 mmol) in DCM (10 mL) was added 4 M HCl in 1,4-dioxane (2 mL, 8 mmol) at 0° C., then the solution was stirred at room temperature for 4 h. The reaction mixture was concentrated under reduced pressure, yielding 1-(piperidin-3-yl)ethanone hydrochloride (2) (500 mg, crude) as a brown residue, which was taken forward to the next step without further purification.

### Step 2: Preparation of 3-(2-(3-acetylpiperidin-1-yl)-2-oxoethyl)-6-phenyl-2H-1,3-oxazin-4(3H)-one (SR71)

To a solution of 2-(4-oxo-6-phenyl-2H-1,3-oxazin-3(4H)-yl)acetic acid (SR462) (200 mg, 0.858 mmol) and 1-(piperidin-3-yl)ethanone hydrochloride (2) (489 mg, 2.98 mmol) in DMF (2 mL) was added DIPEA (0.44 mL, 3 mmol) and HATU (484 mg, 1.27 mmol) at 0° C. The reaction mixture was allowed to stir while warming gradually to room temperature for 4 h. The reaction crude was purified by reverse phase column chromatography (5%-100% ACN-H₂O) yielding compound 3-(2-(3-acetylpiperidin-1-yl)-2-oxoethyl)-6-phenyl-2H-1,3-oxazin-4(3H)-one (SR71) (100 mg, 34% yield) as a white solid. ¹HNMR (600 MHz, CDCl₃) δ 7.72-7.71 (d, 2H), 7.48-7.42 (m, 3H), 5.97 (s, 1H), 5.53-5.47 (m, 1H), 5.39-5.36 (m, 1H), 4.78-4.08 (m, 3H), 3.78-3.75 (d, 1H), 3.40-2.89 (m, 2H), 2.63-2.58 (m, 1H), 2.21-2.03 (m, 4H), 1.87-1.70 (m, 2H). MS (M+H)⁺ 343.2 (m/z). HPLC: 97.03% AUC.

### Example 7

### Synthesis of SR70

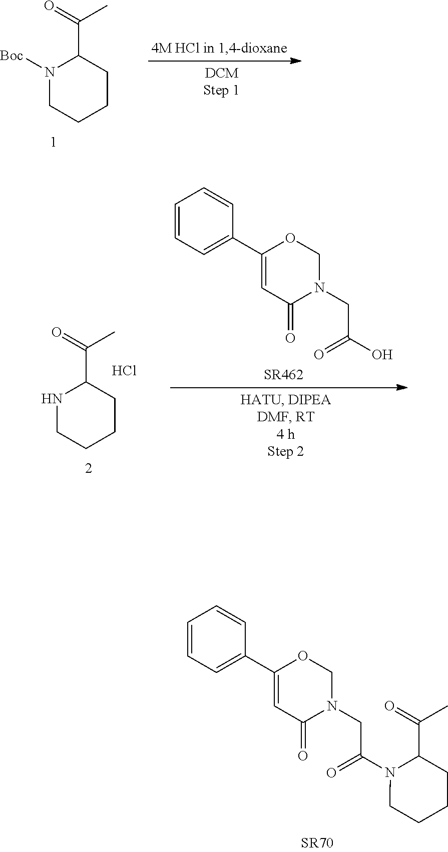

### Step 1: Preparation of 1-(piperidin-2-yl)ethanone hydrochloride (2)

To a stirring solution of tert-butyl 2-acetylpiperidine-1-carboxylate (1) (500 mg, 2.20 mmol) in DCM (10 mL) was added 4 M HCl in 1,4-dioxane (2 mL, 8 mmol) at 0° C., then the solution was stirred at room temperature for 4 h. The reaction mixture was concentrated under reduced pressure, yielding 1-(piperidin-2-yl)ethanone hydrochloride (2) (500 mg, crude) as a brown residue, which was taken forward to the next step without further purification.

### Step 2: Preparation of 3-(2-(2-acetylpiperidin-1-yl)-2-oxoethyl)-6-phenyl-2H-1,3-oxazin-4(3H)-one (SR70)

To a solution of 2-(4-oxo-6-phenyl-2H-1,3-oxazin-3(4H)-yl)acetic acid (SR462) (200 mg, 0.858 mmol) and 1-(piperidin-2-yl)ethanone hydrochloride (2) (489 mg, 2.98 mmol) in DMF (2 mL) was added DIPEA (0.44 mL, 3 mmol) and HATU (484 mg, 1.27 mmol) at 0° C. The reaction mixture was allowed to stir while warming gradually to room temperature for 4 h. The reaction crude was purified by reverse phase column chromatography (5%-100% ACN-H₂O) yielding compound 3-(2-(2-acetylpiperidin-1-yl)-2-oxoethyl)-6-phenyl-2H-1,3-oxazin-4(3H)-one (SR70) (90 mg, 31% yield) as a white solid. ¹HNMR (600 MHz, methanol-d₄) δ 7.78 (d, 2H), 7.52 (m, 1H), 7.47 (t, 2H), 6.00 (s, 1H), 5.46-5.40 (m, 2H), 5.08 (brs, 0.80H), 4.73-4.40 (m, 2.36H), 4.21 (d, 0.2H), 3.81 (d, 0.8H), 3.22 (t, 0.9H), 2.67 (t, 0.2H), 2.42 (d, 0.21H), 2.31-2.27 (m, 1.48H), 2.17 (s, 2.44H), 1.87 (t, 0.22H), 1.84 (brs, 3H), 1.59 (q, 0.86H), 1.48-1.30 (m, 1.3H); signals consistent with rotamer mixture. MS (M+H)⁺ 342.86 (m/z). HPLC: 99.6% AUC.

### Example 8

### Synthesis of SR72

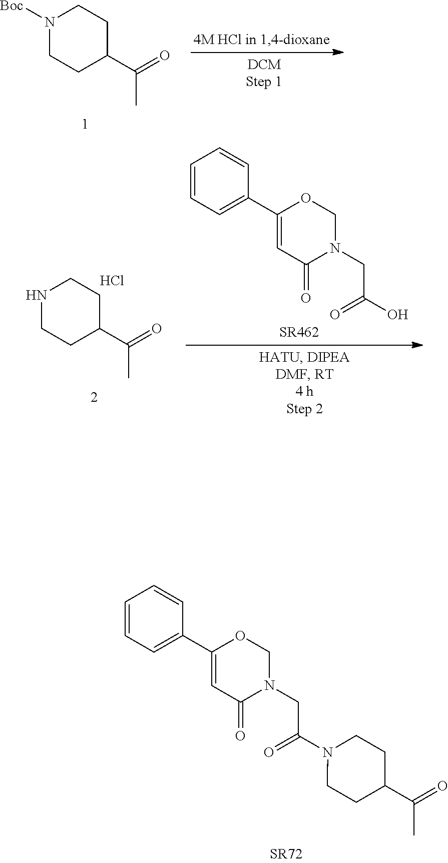

### Step 1: Preparation of 1-(piperidin-4-yl)ethanone hydrochloride (2)

To a stirring solution of tert-butyl 4-acetylpiperidine-1-carboxylate (1) (500 mg, 2.20 mmol) in DCM (10 mL) was added 4 M HCl in 1,4-dioxane (2 mL, 8 mmol) at 0° C., then the solution was stirred at room temperature for 4 h. The reaction mixture was concentrated under reduced pressure, yielding 1-(piperidin-4-yl)ethanone hydrochloride (2) (500 mg, crude) as a brown residue, which was taken forward to the next step without further purification.

### Step 2: Preparation of 3-(2-(4-acetylpiperidin-1-yl)-2-oxoethyl)-6-phenyl-2H-1,3-oxazin-4(3H)-one (SR72)

To a solution of 2-(4-oxo-6-phenyl-2H-1,3-oxazin-3(4H)-yl)acetic acid (SR462) (200 mg, 0.858 mmol) and 1-(piperidin-4-yl)ethanone hydrochloride (2) (489 mg, 2.98 mmol) in DMF (2 mL) was added DIPEA (0.44 mL, 3 mmol) and HATU (484 mg, 1.27 mmol) at 0° C. The reaction mixture was allowed to stir while warming gradually to room temperature for 4 h. The reaction crude was purified by reverse phase column chromatography (5%-100% ACN-H₂O) yielding compound 3-(2-(4-acetylpiperidin-1-yl)-2-oxoethyl)-6-phenyl-2H-1,3-oxazin-4(3H)-one (SR72) (110 mg, 37% yield) as a white solid. ¹HNMR (600 MHz, CDCl₃) δ 7.71 (d, 2H), 7.49-7.46 (m, 1H), 7.44-7.41 (m, 2H), 5.97 (s, 1H), 5.46 (d, 1H), 5.40 (d, 1H), 4.48-4.45 (m, 2H), 4.29 (d, 1H), 3.88 (d, 1H), 3.17 (t, 1H), 2.82 (t, 1H), 2.58 (t, 1H), 2.19 (s, 3H), 1.95 (d, 2H), 1.68-1.64 (m, 1H), 1.57 (q, 1H). MS (M+H)⁺ 342.94 (m/z). HPLC: 99.0% AUC.

### Example 9

### Synthesis of SR459

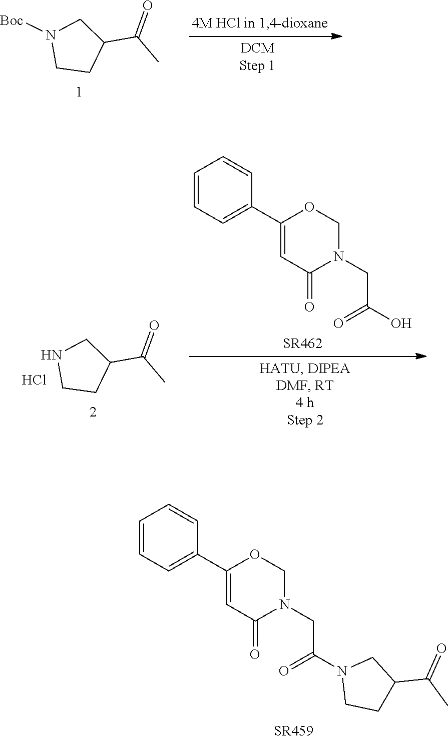

### Step 1: Preparation of 1-(pyrrolidin-3-yl)ethanone hydrochloride (2)

To a stirring solution of tert-butyl 3-acetylpyrrolidine-1-carboxylate (1) (500 mg, 2.34 mmol) in DCM (10 mL) was added 4 M HCl in 1,4-dioxane (2 mL, 8 mmol) at 0° C., then the solution was stirred at rt for 4 h. The reaction mixture was concentrated under reduced pressure, yielding 1-(pyrrolidin-3-yl)ethanone hydrochloride (2) (500 mg, crude) as a brown residue, which was taken forward to the next step without further purification.

### Step 2: Preparation of 3-(2-(3-acetylpyrrolidin-1-yl)-2-oxoethyl)-6-phenyl-2H-1,3-oxazin-4(3H)-one (SR459)

To a solution of 2-(4-oxo-6-phenyl-2H-1,3-oxazin-3(4H)-yl)acetic acid (SR462) (200 mg, 0.858 mmol) and 1-(piperidin-3-yl)ethanone hydrochloride (2) (449 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The reaction crude was purified by reverse phase column chromatography (5%-100% ACN-H₂O) yielding compound 3-(2-(3-acetylpyrrolidin-1-yl)-2-oxoethyl)-6-phenyl-2H-1,3-oxazin-4(3H)-one (SR459) (100 mg, 36% yield) as a white solid. ¹HNMR (600 MHz, CDCl₃) δ 7.72-7.70 (d, 2H), 7.49-7.42 (m, 3H), 5.96 (s, 1H), 5.52-5.51 (m, 1H), 5.42-5.41 (m, 1H), 4.53-4.41 (m, 1H), 4.09-4.07 (m, 1H), 3.78-3.53 (m, 4H), 3.30-3.16 (m, 1H), 2.26-2.20 (m, 5H). MS (M+H)⁺ 328.98 (m/z). HPLC: 96.8% AUC.

### Example 10

### Synthesis of SR459-S

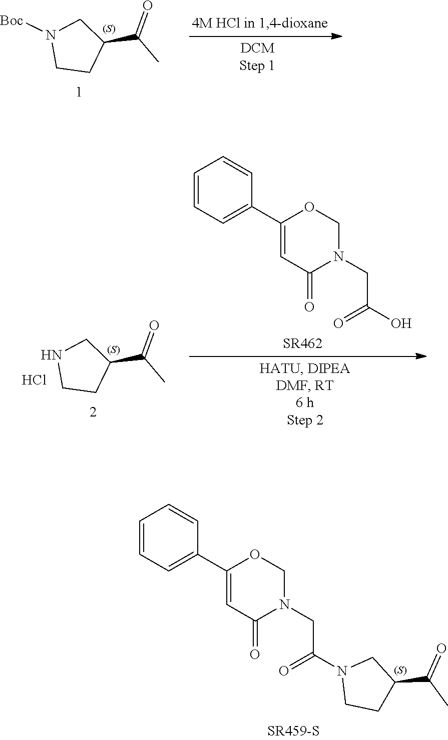

Step 1: Preparation of (S)-1-(pyrrolidin-3-yl)ethanone hydrochloride (2)

To a stirring solution of (S)-tert-butyl 3-acetylpyrrolidine-1-carboxylate (1) (500 mg, 2.34 mmol) in DCM (10 mL) was added 4 M HCl in 1,4-dioxane (2 mL, 8 mmol) at 0° C., then the solution was stirred at rt for 4 h. The reaction mixture was concentrated under reduced pressure, yielding (S)-1-(pyrrolidin-3-yl)ethanone hydrochloride (2) (500 mg, crude) as a brown residue, which was taken forward to the next step without further purification.

### Step 2: Preparation of (S)-3-(2-(3-acetylpyrrolidin-1-yl)-2-oxoethyl)-6-phenyl-2H-1,3-oxazin-4(3H)-one (SR459-S)

To a solution of 2-(4-oxo-6-phenyl-2H-1,3-oxazin-3(4H)-yl)acetic acid (SR462) (200 mg, 0.858 mmol) and (S)-1-(pyrrolidin-3-yl)ethanone hydrochloride (2) (449 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 6 h. The reaction crude was purified by reverse phase column chromatography (5%-100% ACN-H₂O) yielding compound (S)-3-(2-(3-acetylpyrrolidin-1-yl)-2-oxoethyl)-6-phenyl-2H-1,3-oxazin-4(3H)-one (SR459-S) (100 mg, 36% yield) as an off-white solid. ¹HNMR (600 MHz, CDCl₃) δ 7.72-7.70 (d, 2H), 7.49-7.42 (m, 3H), 5.96 (s, 1H), 5.52-5.51 (m, 1H), 5.42-5.41 (m, 1H), 4.53-4.41 (m, 1H), 4.09-4.07 (m, 1H), 3.78-3.53 (m, 4H), 3.30-3.16 (m, 1H), 2.26-2.20 (m, 5H). MS (M+H)⁺ 328.91 (m/z). HPLC: 99.8% AUC.

### Example 11

### Synthesis of SR459-R

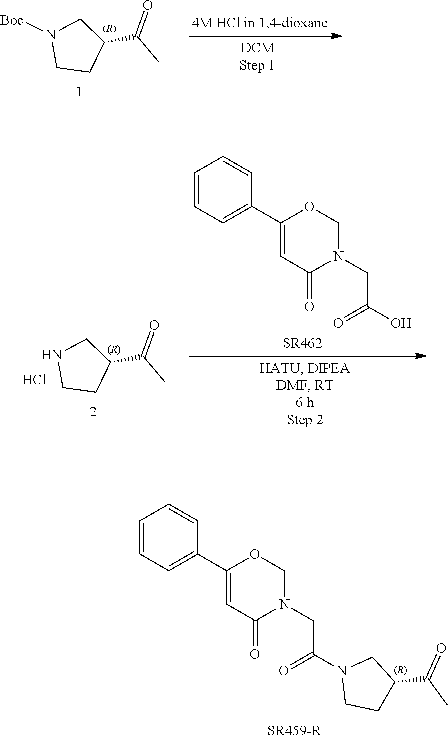

### Step 1: Preparation of (R)-1-(pyrrolidin-3-yl)ethanone hydrochloride (2)

To a stirring solution of (R)-tert-butyl 3-acetylpyrrolidine-1-carboxylate (1) (500 mg, 2.34 mmol) in DCM (10 mL) was added 4 M HCl in 1,4-dioxane (2 mL, 8 mmol) at 0° C., then the solution was stirred at rt for 4 h. The reaction mixture was concentrated under reduced pressure, yielding (R)-1-(pyrrolidin-3-yl)ethanone hydrochloride (2) (500 mg, crude) as a brown residue, which was taken forward to the next step without further purification.

### Step 2: Preparation of (R)-3-(2-(3-acetylpyrrolidin-1-yl)-2-oxoethyl)-6-phenyl-2H-1,3-oxazin-4(3H)-one (SR459-R)

To a solution of 2-(4-oxo-6-phenyl-2H-1,3-oxazin-3(4H)-yl)acetic acid (SR462) (200 mg, 0.858 mmol) and (R)-1-(pyrrolidin-3-yl)ethanone hydrochloride (2) (449 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 6 h. The reaction crude was purified by reverse phase column chromatography (5%-100% ACN-H₂O) yielding compound (R)-3-(2-(3-acetylpyrrolidin-1-yl)-2-oxoethyl)-6-phenyl-2H-1,3-oxazin-4(3H)-one (SR459-R) (100 mg, 36% yield) as an off-white solid. ¹HNMR (600 MHz, CDCl₃) δ 7.72-7.70 (d, 2H), 7.49-7.42 (m, 3H), 5.96 (s, 1H), 5.52-5.51 (m, 1H), 5.42-5.41 (m, 1H), 4.53-4.41 (m, 1H), 4.09-4.07 (m, 1H), 3.78-3.53 (m, 4H), 3.30-3.16 (m, 1H), 2.26-2.20 (m, 5H). MS (M+H)⁺ 328.91 (m/z). HPLC: 97.6% AUC.

### Example 12

### Synthesis of SR466

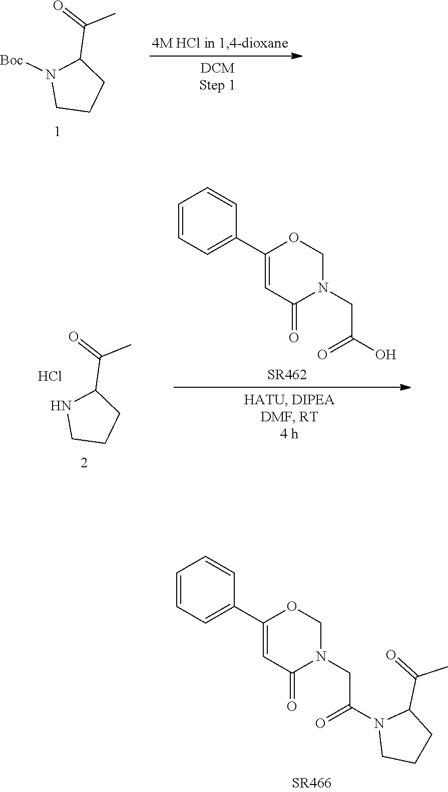

### Step 1: Preparation of 1-(pyrrolidin-2-yl)ethanone hydrochloride (2)

To a stirring solution of tert-butyl 2-acetylpyrrolidine-1-carboxylate (1) (500 mg, 2.34 mmol) in DCM (10 mL) was added 4 M HCl in 1,4-dioxane (2 mL, 8 mmol) at 0° C., then the solution was stirred at rt for 4 h. The reaction mixture was concentrated under reduced pressure, yielding 1-(pyrrolidin-2-yl)ethanone hydrochloride (2) (500 mg, crude) as a brown residue, which was taken forward to the next step without further purification.

### Step 2: Preparation of 3-(2-(2-acetylpyrrolidin-1-yl)-2-oxoethyl)-6-phenyl-2H-1,3-oxazin-4(3H)-one (SR466)

To a solution of 2-(4-oxo-6-phenyl-2H-1,3-oxazin-3(4H)-yl)acetic acid (SR462) (200 mg, 0.858 mmol) and 1-(pyrrolidin-2-yl)ethanone hydrochloride (2) (449 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The reaction crude was purified by reverse phase column chromatography (5%-100% ACN-H₂O) yielding compound 3-(2-(2-acetylpyrrolidin-1-yl)-2-oxoethyl)-6-phenyl-2H-1,3-oxazin-4(3H)-one (SR466) (97 mg, 35% yield) as a brown, beige solid. ¹HNMR (600 MHz, methanol-d₄) δ 7.78 (d, 2H), 7.52 (m, 1H), 7.47 (t, 2H), 5.99 (s, 1H), 5.44 (s, 2H), 4.55 (t, 0.87H), 4.49 (d, 0.87H), 4.37 (t, 1H), 3.84 (d, 0.15H), 3.68 (t, 1.75H), 3.69-3.52 (m, 0.31H), 2.45-2.38 (m, 0.15H), 2.28-2.19 (m, 4.15H), 1.06-1.98 (m, 1.80H), 1.93-1.90 (m, 1.06H); signals consistent with rotamer mixture. MS (M+H)⁺ 328.91 (m/z). HPLC: 99.6% AUC.

### Example 13

### Synthesis of SR466-S

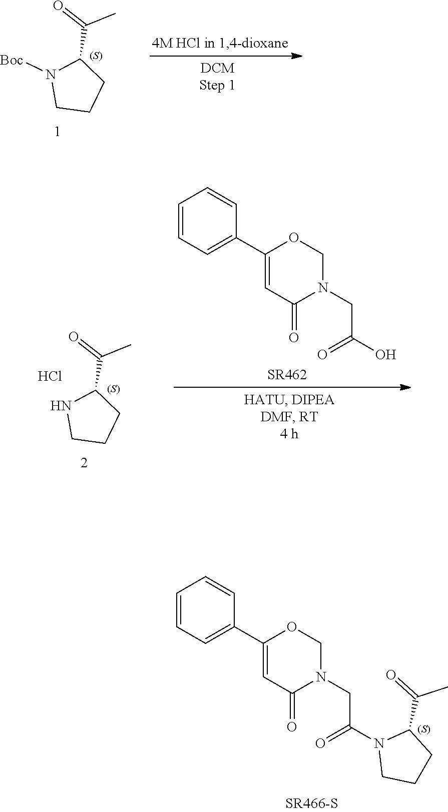

### Step 1: Preparation of (S)-1-(pyrrolidin-2-yl)ethanone hydrochloride (2)

To a stirring solution of (S)-tert-butyl 2-acetylpyrrolidine-1-carboxylate (1) (500 mg, 2.34 mmol) in DCM (10 mL) was added 4 M HCl in 1,4-dioxane (2 mL, 8 mmol) at 0° C., then the solution was stirred at rt for 4 h. The reaction mixture was concentrated under reduced pressure, yielding (S)-1-(pyrrolidin-2-yl)ethanone hydrochloride (2) (500 mg, crude) as a brown residue, which was taken forward to the next step without further purification.

### Step 2: Preparation of (S)-3-(2-(2-acetylpyrrolidin-1-yl)-2-oxoethyl)-6-phenyl-2H-1,3-oxazin-4(3H)-one (SR466-S)

To a solution of 2-(4-oxo-6-phenyl-2H-1,3-oxazin-3(4H)-yl)acetic acid (SR462) (200 mg, 0.858 mmol) and (S)-1-(pyrrolidin-2-yl)ethanone hydrochloride (2) (449 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The reaction crude was purified by reverse phase column chromatography (5%-100% ACN-H₂O) yielding compound (S)-3-(2-(2-acetylpyrrolidin-1-yl)-2-oxoethyl)-6-phenyl-2H-1,3-oxazin-4(3H)-one (SR466-S) (90 mg, 32% yield) as a brown, beige solid. ¹HNMR (600 MHz, methanol-d₄) δ 7.78 (d, 2H), 7.52 (m, 1H), 7.47 (t, 2H), 5.99 (s, 1H), 5.44 (s, 2H), 4.55 (t, 0.87H), 4.49 (d, 0.87H), 4.37 (t, 1H), 3.84 (d, 0.15H), 3.68 (t, 1.75H), 3.69-3.52 (m, 0.31H), 2.45-2.38 (m, 0.15H), 2.28-2.19 (m, 4.15H), 1.06-1.98 (m, 1.80H), 1.93-1.90 (m, 1.06H); signals consistent with rotamer mixture. MS (M+H)⁺ 328.91 (m/z). HPLC: 99.7% AUC.

### Example 14

### Synthesis of SR466-R

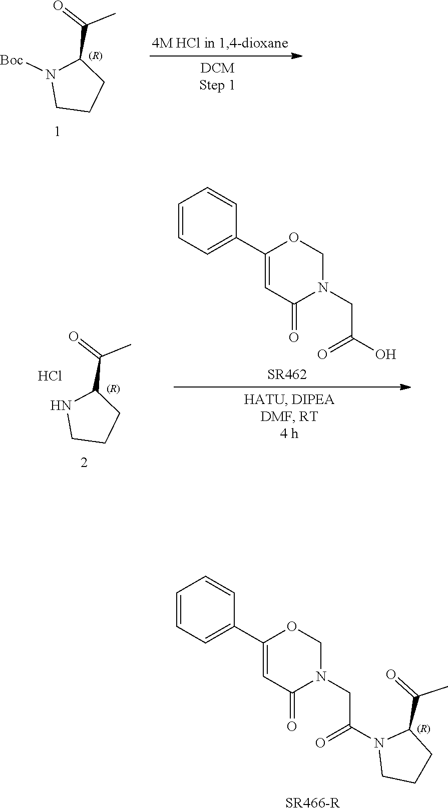

### Step 1: Preparation of (R)-1-(pyrrolidin-2-yl)ethanone hydrochloride (2)

To a stirring solution of (R)-tert-butyl 2-acetylpyrrolidine-1-carboxylate (1) (500 mg, 2.34 mmol) in DCM (10 mL) was added 4 M HCl in 1,4-dioxane (2 mL, 8 mmol) at 0° C., then the solution was stirred at rt for 4 h. The reaction mixture was concentrated under reduced pressure, yielding (R)-1-(pyrrolidin-2-yl)ethanone hydrochloride (2) (500 mg, crude) as a brown residue, which was taken forward to the next step without further purification.

### Step 2: Preparation of (R)-3-(2-(2-acetylpyrrolidin-1-yl)-2-oxoethyl)-6-phenyl-2H-1,3-oxazin-4(3H)-one (SR466-R)

To a solution of 2-(4-oxo-6-phenyl-2H-1,3-oxazin-3(4H)-yl)acetic acid (SR462) (200 mg, 0.858 mmol) and (R)-1-(pyrrolidin-2-yl)ethanone hydrochloride (2) (449 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The reaction crude was purified by reverse phase column chromatography (5%-100% ACN-H₂O) yielding compound (R)-3-(2-(2-acetylpyrrolidin-1-yl)-2-oxoethyl)-6-phenyl-2H-1,3-oxazin-4(3H)-one (SR466-R) (92 mg, 33% yield) as a brown, beige solid. ¹HNMR (600 MHz, methanol-d₄) δ 7.78 (d, 2H), 7.52 (m, 1H), 7.47 (t, 2H), 5.99 (s, 1H), 5.44 (s, 2H), 4.55 (t, 0.87H), 4.49 (d, 0.87H), 4.37 (t, 1H), 3.84 (d, 0.15H), 3.68 (t, 1.75H), 3.69-3.52 (m, 0.31H), 2.45-2.38 (m, 0.15H), 2.28-2.19 (m, 4.15H), 1.06-1.98 (m, 1.80H), 1.93-1.90 (m, 1.06H); signals consistent with rotamer mixture. MS (M+H)+328.91 (m/z). HPLC: 99.4% AUC.

Additional compounds can be prepared by using similar synthesis routes, e.g., using acids with a different amine hydrochloride salt to form the amide. Examples of compounds synthesized in this manner are found below:

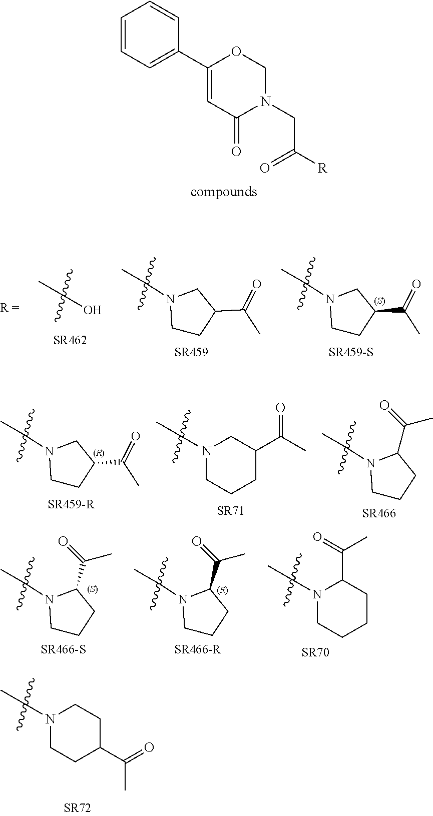

### SR462:

¹HNMR (600 MHz, CDCl₃) δ 7.79-7.78 (d, 2H), 7.53-7.46 (m, 3H), 6.07 (s, 1H), 5.41 (s, 2H), 5.14 (s, 2H). MS (M+H)⁺ 234.2 (m/z). HPLC: 97.1% AUC.

### SR459:

¹HNMR (600 MHz, CDCl₃) δ 7.72-7.70 (d, 2H), 7.49-7.42 (m, 3H), 5.96 (s, 1H), 5.52-5.51 (m, 1H), 5.42-5.41 (m, 1H), 4.53-4.41 (m, 1H), 4.09-4.07 (m, 1H), 3.78-3.53 (m, 4H), 3.30-3.16 (m, 1H), 2.26-2.20 (m, 5H). MS (M+H)⁺ 328.98 (m/z). HPLC: 96.8% AUC.

### SR459-S:

¹HNMR (600 MHz, CDCl₃) δ 7.72-7.70 (d, 2H), 7.49-7.42 (m, 3H), 5.96 (s, 1H), 5.52-5.51 (m, 1H), 5.42-5.41 (m, 1H), 4.53-4.41 (m, 1H), 4.09-4.07 (m, 1H), 3.78-3.53 (m, 4H), 3.30-3.16 (m, 1H), 2.26-2.20 (m, 5H). MS (M+H)⁺ 328.91 (m/z). HPLC: 99.8% AUC.

### SR459-R:

¹HNMR (600 MHz, CDCl₃) δ 7.72-7.70 (d, 2H), 7.49-7.42 (m, 3H), 5.96 (s, 1H), 5.52-5.51 (m, 1H), 5.42-5.41 (m, 1H), 4.53-4.41 (m, 1H), 4.09-4.07 (m, 1H), 3.78-3.53 (m, 4H), 3.30-3.16 (m, 1H), 2.26-2.20 (m, 5H). MS (M+H)⁺ 328.91 (m/z). HPLC: 97.6% AUC.

### SR71:

¹HNMR (600 MHz, CDCl₃) δ 7.72-7.71 (d, 2H), 7.48-7.42 (m, 3H), 5.97 (s, 1H), 5.53-5.47 (m, 1H), 5.39-5.36 (m, 1H), 4.78-4.08 (m, 3H), 3.78-3.75 (d, 1H), 3.40-2.89 (m, 2H), 2.63-2.58 (m, 1H), 2.21-2.03 (m, 4H), 1.87-1.70 (m, 2H). MS (M+H)⁺ 343.2 (m/z). HPLC: 97.03% AUC.

### SR70:

¹HNMR (600 MHz, methanol-d₄) δ 7.78 (d, 2H), 7.52 (m, 1H), 7.47 (t, 2H), 6.00 (s, 1H), 5.46-5.40 (m, 2H), 5.08 (brs, 0.80H), 4.73-4.40 (m, 2.36H), 4.21 (d, 0.2H), 3.81 (d, 0.8H), 3.22 (t, 0.9H), 2.67 (t, 0.2H), 2.42 (d, 0.21H), 2.31-2.27 (m, 1.48H), 2.17 (s, 2.44H), 1.87 (t, 0.22H), 1.84 (brs, 3H), 1.59 (q, 0.86H), 1.48-1.30 (m, 1.3H); signals consistent with rotamer mixture. MS (M+H)⁺ 342.86 (m/z). HPLC: 99.6% AUC.

### SR72:

¹HNMR (600 MHz, CDCl₃) δ 7.71 (d, 2H), 7.49-7.46 (m, 1H), 7.44-7.41 (m, 2H), 5.97 (s, 1H), 5.46 (d, 1H), 5.40 (d, 1H), 4.48-4.45 (m, 2H), 4.29 (d, 1H), 3.88 (d, 1H), 3.17 (t, 1H), 2.82 (t, 1H), 2.58 (t, 1H), 2.19 (s, 3H), 1.95 (d, 2H), 1.68-1.64 (m, 1H), 1.57 (q, 1H). MS (M+H)⁺ 342.94 (m/z). HPLC: 99.0% AUC.

### SR466:

¹HNMR (600 MHz, methanol-d₄) δ 7.78 (d, 2H), 7.52 (m, 1H), 7.47 (t, 2H), 5.99 (s, 1H), 5.44 (s, 2H), 4.55 (t, 0.87H), 4.49 (d, 0.87H), 4.37 (t, 1H), 3.84 (d, 0.15H), 3.68 (t, 1.75H), 3.69-3.52 (m, 0.31H), 2.45-2.38 (m, 0.15H), 2.28-2.19 (m, 4.15H), 1.06-1.98 (m, 1.80H), 1.93-1.90 (m, 1.06H); signals consistent with rotamer mixture. MS (M+H)⁺ 328.91 (m/z).

HPLC: 99.6% AUC.

### SR466-S:

¹HNMR (600 MHz, methanol-d₄) δ 7.78 (d, 2H), 7.52 (m, 1H), 7.47 (t, 2H), 5.99 (s, 1H), 5.44 (s, 2H), 4.55 (t, 0.87H), 4.49 (d, 0.87H), 4.37 (t, 1H), 3.84 (d, 0.15H), 3.68 (t, 1.75H), 3.69-3.52 (m, 0.31H), 2.45-2.38 (m, 0.15H), 2.28-2.19 (m, 4.15H), 1.06-1.98 (m, 1.80H), 1.93-1.90 (m, 1.06H); signals consistent with rotamer mixture. MS (M+H)⁺ 328.91 (m/z). HPLC: 99.7% AUC.

### SR466-R:

¹HNMR (600 MHz, methanol-d₄) δ 7.78 (d, 2H), 7.52 (m, 1H), 7.47 (t, 2H), 5.99 (s, 1H), 5.44 (s, 2H), 4.55 (t, 0.87H), 4.49 (d, 0.87H), 4.37 (t, 1H), 3.84 (d, 0.15H), 3.68 (t, 1.75H), 3.69-3.52 (m, 0.31H), 2.45-2.38 (m, 0.15H), 2.28-2.19 (m, 4.15H), 1.06-1.98 (m, 1.80H), 1.93-1.90 (m, 1.06H); signals consistent with rotamer mixture. MS (M+H)⁺ 328.91 (m/z). HPLC: 99.4% AUC.

### Example 15

### Additional Approaches for Synthesis

### Synthesis of SR4722

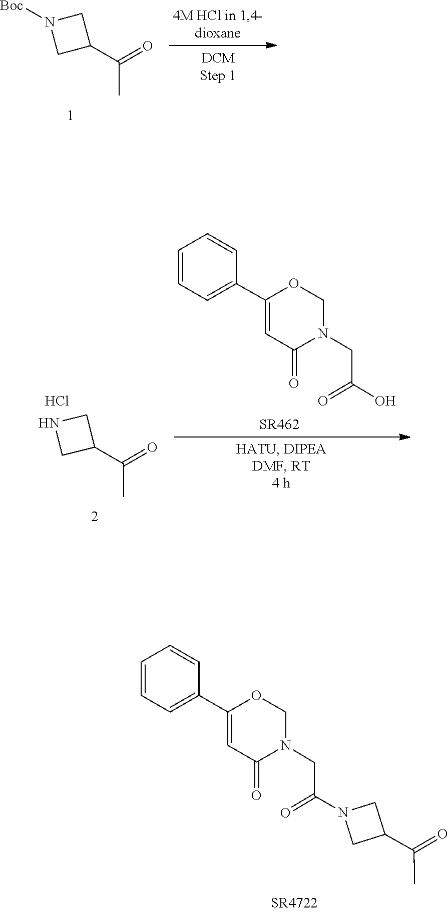

SR4722 is prepared by taking tert-butyl 3-acetylazetidine-1-carboxylate (1) in DCM and treating it with 4 M HCl in 1,4-dioxane to obtain 1-(azetidin-3-yl)ethanone hydrochloride (2). Compound 2 will then be taken with 2-(4-oxo-6-phenyl-2H-1,3-oxazin-3(4H)-yl)acetic acid (SR462) in DMF and will be treated with DIPEA and HATU at room temperature and will be purified to yield 3-(2-(3-acetylazetidin-1-yl)-2-oxoethyl)-6-phenyl-2H-1,3-oxazin-4(3H)-one (SR4722).

### Synthesis of SR7280

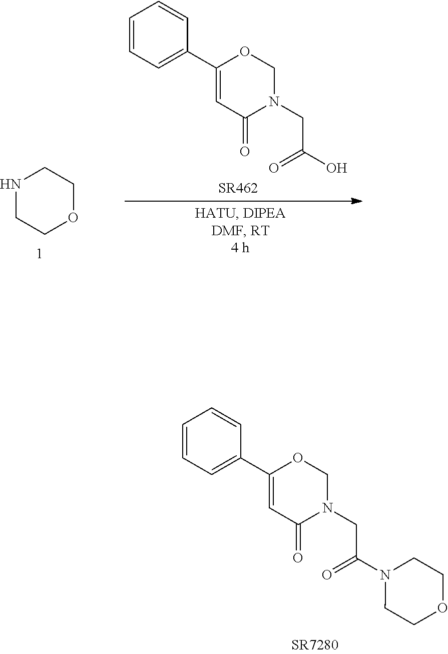 [SMILES: C1COCCN1 ; O=C(O)CN1COC(c2ccccc2)=CC1=O ; O=C1C=C(c2ccccc2)OCN1CC(=O)N1CCOCC1]

SR7280 is prepared by taking morpholine (1) with 2-(4-oxo-6-phenyl-2H-1,3-oxazin-3(4H)-yl)acetic acid (SR462) in DMF and treating it with DIPEA and HATU at room temperature and will be purified to yield 3-(2-morpholino-2-oxoethyl)-6-phenyl-2H-1,3-oxazin-4(3H)-one (SR7280).

### Synthesis of SR7255

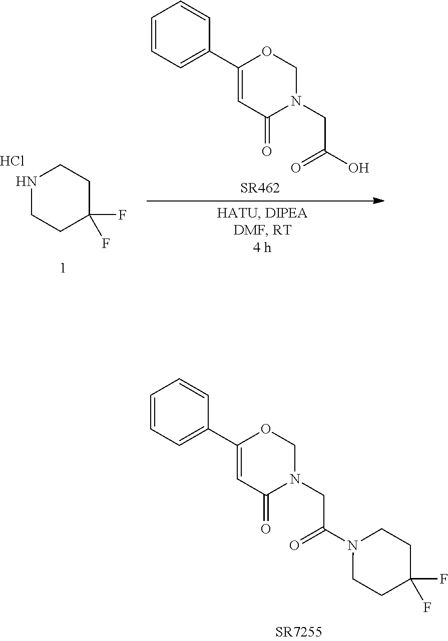 [SMILES: Cl ; FC1(F)CCNCC1 ; O=C(O)CN1COC(c2ccccc2)=CC1=O ; O=C1C=C(c2ccccc2)OCN1CC(=O)N1CCC(F)(F)CC1]

SR7255 is prepared by taking 4,4-difluoropiperidine hydrochloride (1) with 2-(4-oxo-6-phenyl-2H-1,3-oxazin-3(4H)-yl)acetic acid (SR462) in DMF and treating it with DIPEA and HATU at room temperature and will be purified to yield 3-(2-(4,4-difluoropiperidin-1-yl)-2-oxoethyl)-6-phenyl-2H-1,3-oxazin-4(3H)-one (SR7255).

### Synthesis of SR5955

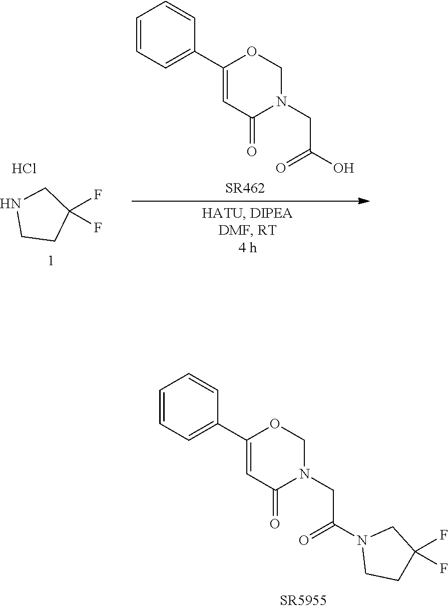 [SMILES: O=C(O)CN1COC(c2ccccc2)=CC1=O ; Cl ; FC1(F)CCNC1 ; O=C1C=C(c2ccccc2)OCN1CC(=O)N1CCC(F)(F)C1]

SR5955 is prepared by taking 3,3-difluoropyrrolidine hydrochloride (1) with 2-(4-oxo-6-phenyl-2H-1,3-oxazin-3(4H)-yl)acetic acid (SR462) in DMF and treating it with DIPEA and HATU at room temperature and will be purified to yield 3-(2-(3,3-difluoropyrrolidin-1-yl)-2-oxoethyl)-6-phenyl-2H-1,3-oxazin-4(3H)-one (SR5955).

### Synthesis of SR7254

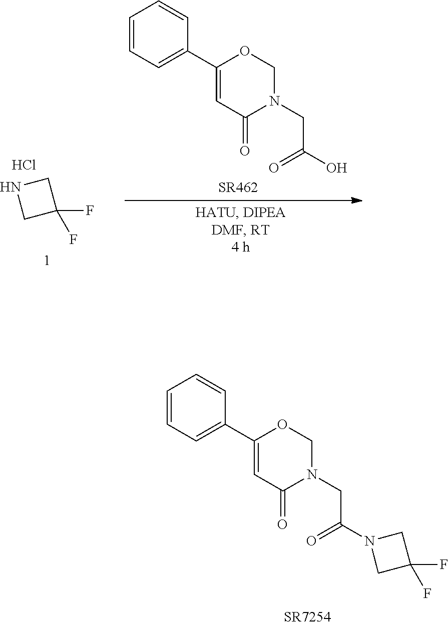 [SMILES: Cl ; O=C(O)CN1COC(c2ccccc2)=CC1=O ; FC1(F)CNC1 ; O=C1C=C(c2ccccc2)OCN1CC(=O)N1CC(F)(F)C1]

SR7254 is prepared by taking 3,3-difluoroazetidine hydrochloride (1) with 2-(4-oxo-6-phenyl-2H-1,3-oxazin-3(4H)-yl)acetic acid (SR462) in DMF and treating it with DIPEA and HATU at room temperature and will be purified to yield 3-(2-(3,3-difluoroazetidin-1-yl)-2-oxoethyl)-6-phenyl-2H-1,3-oxazin-4(3H)-one (SR7254). Synthesis of SR4721

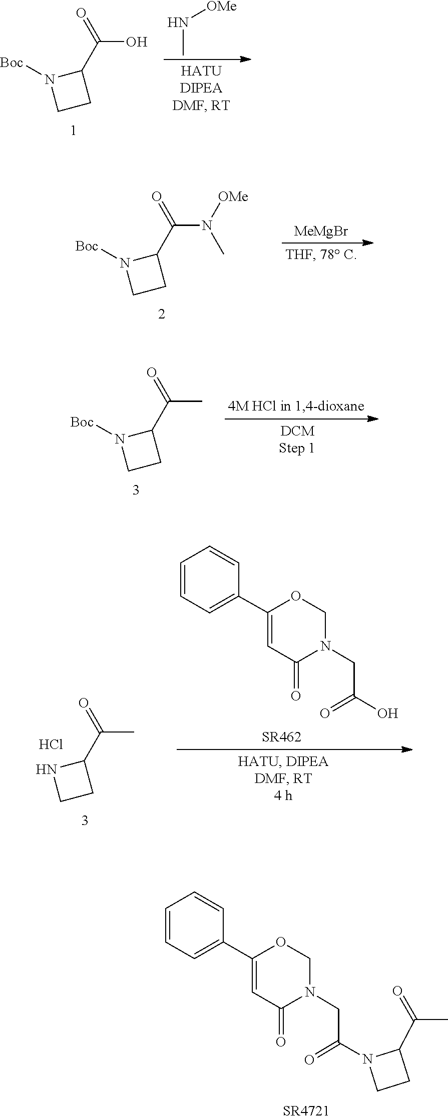

SR4721 is prepared by taking 1-(tert-butoxycarbonyl)azetidine-2-carboxylic acid (1) and N,O-dimethylhydroxylamine in DMF and treating it DIPEA and HATU at room temperature and will be purified to yield tert-butyl 2-(methoxy(methyl)carbamoyl)azetidine-1-carboxylate (2). Intermediate 2 will then be treated with methyl magnesium bromide in THF at −78° C. and will be quenched and purified yielding tert-butyl 2-acetylazetidine-1-carboxylate (3). Intermediate 3 will then be treated with 4 M HCl in 1,4-dioxane to obtain 1-(azetidin-2-yl)ethanone hydrochloride (4). Compound 4 will then be taken with 2-(4-oxo-6-phenyl-2H-1,3-oxazin-3(4H)-yl)acetic acid (SR462) in DMF and will be treated with DIPEA and HATU at room temperature and will be purified to yield 3-(2-(3-acetylazetidin-1-yl)-2-oxoethyl)-6-phenyl-2H-1,3-oxazin-4(3H)-one (SR4722).

### Example 16

### Additional Approaches for Synthesis

### Synthesis of SR401 and SR438-R:

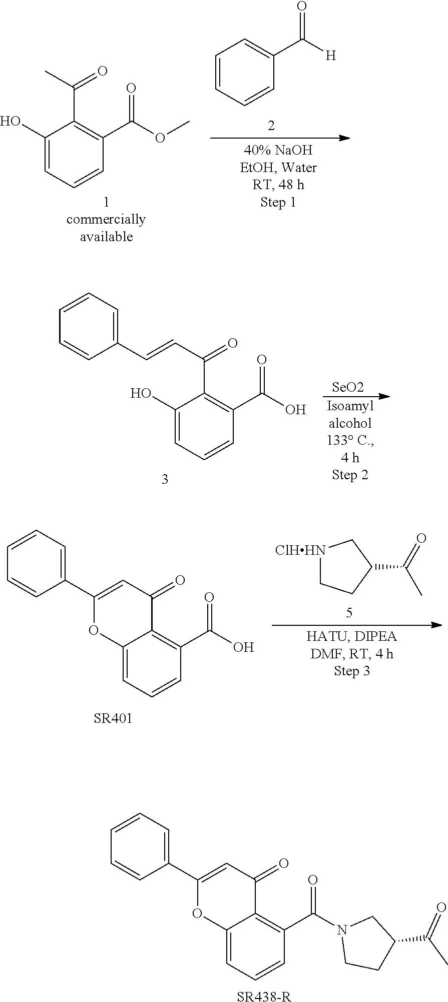

### Step 1: Preparation of 2-cinnamoyl-3-hydroxybenzoic acid (3)

To a solution of methyl 2-acetyl-3-hydroxybenzoate (commercially available) (10 g, 51.54 mmol) and benzaldehyde (10.1 g, 95.36 mmol) in Ethanol (250 mL) was added 40% NaOH (120 mL) at rt. The reaction mixture was allowed to room temperature for 48 h. Then the reaction mixture was distilled completely, acidified with concentrated HCl to pH 2-3, and extracted with EtOAc. The combined organic layer was concentrated in vacuo and the crude residue was washed with 20% EtOAc/hexane solution, yielding 2-cinnamoyl-3-hydroxybenzoic acid (3) (10.0 g, 72%) as an off-white solid. This material was taken into the next step without further purification.

### Step 2: Preparation of 4-oxo-2-phenyl-4H-chromene-5-carboxylic acid (SR401)

To a solution of 2-cinnamoyl-3-hydroxybenzoic acid (3) (10 g, 37.34 mmol) in isoamyl alcohol (100 mL) was added SeO₂ (8.28 g, 74.62 mmol), and the reaction mixture was stirred at 133° C. for 4 h. The reaction mixture was filtered and washed with MTBE (150 mL). Then the solids were taken in to 250 mL flask with water (150 mL) and the mixture was stirred for 1 h, then filtered and dried, yielding 4-oxo-2-phenyl-4H-chromene-5-carboxylic acid (SR401) (3.50 g, 35%) as a grey, fluffy solid. ¹HNMR (600 MHz, DMSO-d₆) δ 8.15-8.13 (d, 2H), 7.88-7.84 (m, 2H), 7.65-7.59 (s, 3H), 7.44-7.40 (m, 1H), 7.09 (s, 1H). MS (M+H)⁺ 267.1 (m/z). HPLC: 99.9% AUC.

### Step 3: Preparation of (R)-5-(3-acetylpyrrolidine-1-carbonyl)-2-phenyl-4H-chromen-4-one (SR438-R)

To a solution of 4-oxo-2-phenyl-4H-chromene-5-carboxylic acid (SR401) (200 mg, 0.752 mmol) and (R)-1-(pyrrolidin-2-yl)ethanone hydrochloride (2) (168 mg, 1.12 mmol) in DMF (2 mL) was added DIPEA (0.38 mL, 2.25 mmol) and HATU (427 mg, 1.12 mmol) at 0° C. The reaction mixture was allowed to stir while warming gradually to room temperature for 4 h. The crude residue was purified via reverse phase column chromatography (5%-100% ACN-H₂O), yielding (R)-5-(3-acetylpyrrolidine-1-carbonyl)-2-phenyl-4H-chromen-4-one (SR438-R) (50 mg, 18% yield) as a brown, beige solid. ¹HNMR (600 MHz, CDCl₃) δ 7.94 (d, 2H), 7.75-7.31 (m, 6H), 6.78 (s, 1H), 4.24-3.78 (m, 2H), 3.36-3.12 (m, 3H), 2.41-2.05 (m, 5H); signals consistent with a mixture of rotamers. MS (M+H)⁺ 362.11 (m/z). HPLC: 99.9% AUC.

### Synthesis of SR438-S

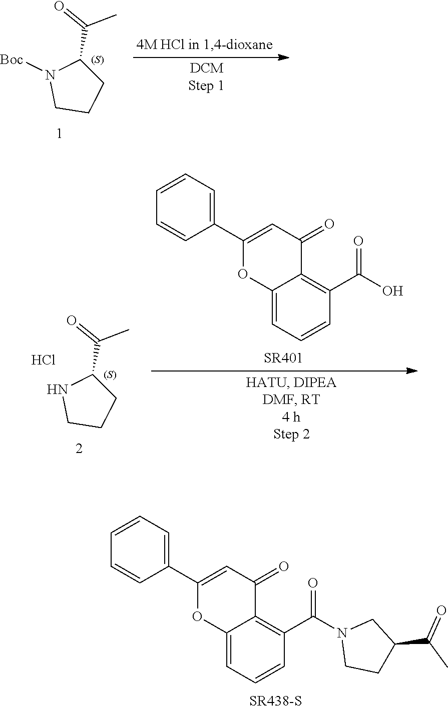

### Step 1: Preparation of (S)-1-(pyrrolidin-2-yl)ethanone hydrochloride (2)

To a stirring solution of (S)-tert-butyl 2-acetylpyrrolidine-1-carboxylate (1) (500 mg, 2.34 mmol) in DCM (10 mL) was added 4 M HCl in 1,4-dioxane (2 mL, 8 mmol) at 0° C., then the solution was stirred at rt for 4 h. The reaction mixture was concentrated under reduced pressure, yielding (S)-1-(pyrrolidin-2-yl)ethanone hydrochloride (2) (500 mg, crude) as a brown residue, which was taken forward to the next step without further purification.

### Step 2: Preparation of (S)-5-(3-acetylpyrrolidine-1-carbonyl)-2-phenyl-4H-chromen-4-one (SR438-S)

To a solution of 4-oxo-2-phenyl-4H-chromene-5-carboxylic acid (SR401) (200 mg, 0.752 mmol) and (S)-1-(pyrrolidin-2-yl)ethanone hydrochloride (2) (168 mg, 1.12 mmol) in DMF (2 mL) was added DIPEA (0.38 mL, 2.25 mmol) and HATU (427 mg, 1.12 mmol) at 0° C. The reaction mixture was allowed to stir while warming gradually to room temperature for 4 h. The crude residue was purified by reverse phase column chromatography (5%-100% ACN-H₂O), yielding (S)-5-(3-acetylpyrrolidine-1-carbonyl)-2-phenyl-4H-chromen-4-one (SR438-S) (55 mg, 20% yield) as a brown, beige solid. ¹HNMR (600 MHz, CDCl₃) δ 7.94 (d, 2H), 7.75-7.31 (m, 6H), 6.78 (s, 1H), 4.24-3.78 (m, 2H), 3.36-3.12 (m, 3H), 2.41-2.05 (m, 5H); signals consistent with a mixture of rotamers. MS (M+H)⁺ 362.11 (m/z). HPLC: 99.8% AUC.

### Synthesis of Racemic SR438

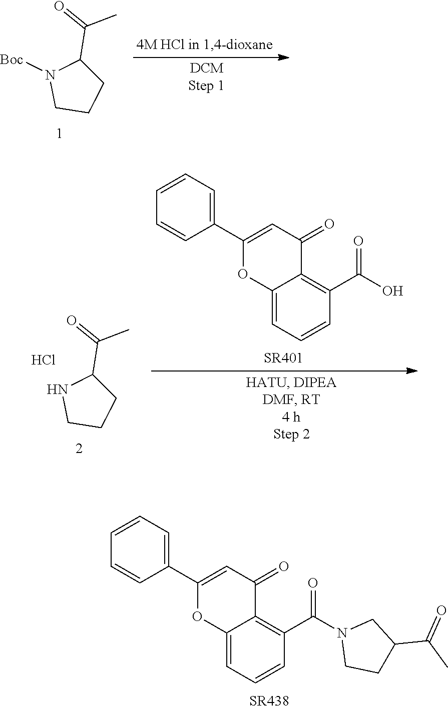

### Step 1: Preparation of 1-(pyrrolidin-2-yl)ethanone hydrochloride (2)

To a stirring solution of tert-butyl 2-acetylpyrrolidine-1-carboxylate (1) (500 mg, 2.34 mmol) in DCM (10 mL) was added 4 M HCl in 1,4-dioxane (2 mL, 8 mmol) at 0° C., then the solution was stirred at rt for 4 h. The reaction mixture was concentrated under reduced pressure, yielding 1-(pyrrolidin-2-yl)ethanone hydrochloride (2) (500 mg, crude) as a brown residue, which was taken forward to the next step without further purification.

### Step 2: Preparation of 5-(3-acetylpyrrolidine-1-carbonyl)-2-phenyl-4H-chromen-4-one (SR438-S)

To a solution of 4-oxo-2-phenyl-4H-chromene-5-carboxylic acid (SR401) (200 mg, 0.752 mmol) and 1-(pyrrolidin-2-yl)ethanone hydrochloride (2) (168 mg, 1.12 mmol) in DMF (2 mL) was added DIPEA (0.38 mL, 2.25 mmol) and HATU (427 mg, 1.12 mmol) at 0° C. The reaction mixture was allowed to stir while warming gradually to room temperature for 4 h. The crude residue was purified by reverse phase column chromatography (5%-100% ACN-H₂O), yielding 5-(3-acetylpyrrolidine-1-carbonyl)-2-phenyl-4H-chromen-4-one (SR438) (8 mg, 5% yield) as an off-white solid. ¹HNMR (600 MHz, DMSO-d₆) δ 8.13-8.12 (d, 2H), 7.86-7.84 (m, 2H), 7.62-7.60 (m, 3H), 7.29-7.25 (m, 1H), 7.02-7.01 (s, 1H), 3.79-3.45 (m, 2H), 3.29-2.90 (m, 3H), 2.22-2.20 (m, 2H), 2.09-2.05 (m, 3H); signals consistent with mixture of enantiomers. (M+H)⁺ 361.39 (m/z). HPLC: 99.2% AUC.

### Synthesis of SR4386

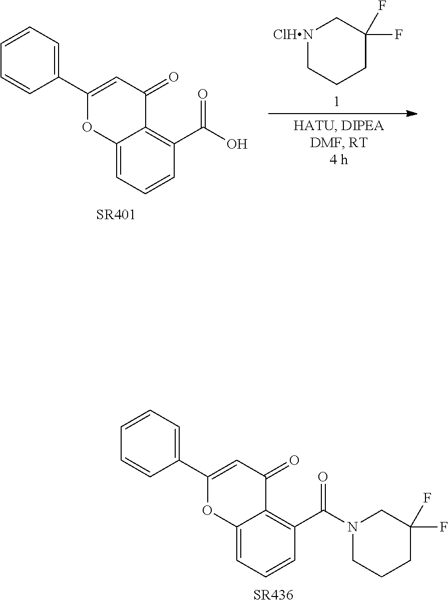

### Preparation of 5-(3,3-difluoropiperidine-1-carbonyl)-2-phenyl-4H-chromen-4-one (SR4386)

To a solution of 4-oxo-2-phenyl-4H-chromene-5-carboxylic acid (SR401) (200 mg, 0.752 mmol) and 3,3-difluoropiperidine hydrochloride (1) (176 mg, 1.12 mmol) in DMF (2 mL) was added DIPEA (0.382 mL, 2.25 mmol) and HATU (427 mg, 1.12 mmol) at 0° C. The reaction mixture was allowed to stir while warming gradually to room temperature for 4 h. The crude residue was purified by reverse phase column chromatography (5%-100% ACN-H₂O), yielding compound 5-(3,3-difluoropiperidine-1-carbonyl)-2-phenyl-4H-chromen-4-one (SR4386) (111 mg, 39% yield) as an off-white solid. ¹HNMR (600 MHz, CDCl₃) δ 7.94-7.93 (d, 2H), 7.78-7.70 (q, 1H), 7.66-7.64 (m, 1H), 7.58-7.54 (m, 3H), 7.33-7.29 (m, 1H), 6.80-6.79 (d, 1H), 4.75-4.72 (m, 1H), 3.50-3.47 (m, 1H), 3.27-3.04 (m, 2H), 2.27-2.18 (m, 1H), 1.98-1.87 (m, 2H), 1.71-1.69 (m, 1H). MS (M+H)*370.12 (m/z). HPLC: 99.0% AUC.

### Synthesis of SR4385

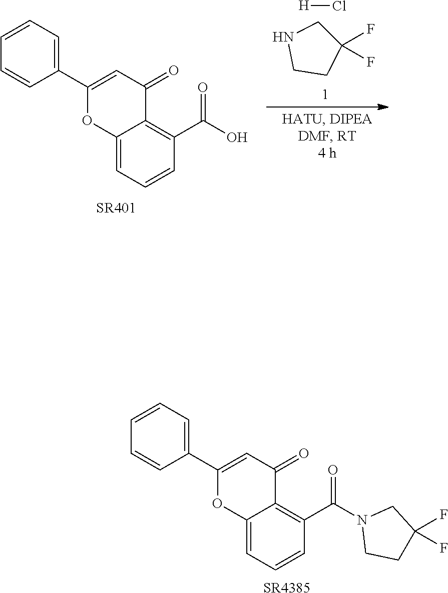

### Preparation of 5-(3,3-difluoropyrrolidine-1-carbonyl)-2-phenyl-4H-chromen-4-one (SR4385)

To a solution of 4-oxo-2-phenyl-4H-chromene-5-carboxylic acid (SR401) (200 mg, 0.752 mmol) and 3,3-difluoropyrrolidine hydrochloride (1) (160 mg, 1.12 mmol) in DMF (2 mL) was added DIPEA (0.382 mL, 2.25 mmol) and HATU (427 mg, 1.12 mmol) at 0° C. The reaction mixture was allowed to stir while warming gradually to room temperature for 4 h. The crude residue was purified by reverse phase column chromatography (5%-100% ACN-H₂O), yielding compound 5-(3,3-difluoropyrrolidine-1-carbonyl)-2-phenyl-4H-chromen-4-one (SR4385) (110 mg, 40% yield) as an off-white solid. ¹HNMR (600 MHz, CDCl₃) δ 7.95-7.93 (d, 2H), 7.78-7.74 (m, 1H), 7.67-7.66 (d, 1H), 7.59-7.55 (m, 3H), 7.33-7.31 (d, 1H), 6.86 (s, 1H), 4.22-4.15 (m, 1H), 4.08-3.87 (m, 1H), 3.43-3.30 (m, 2H), 2.55-2.31 (m, 2H); MS (M+H)⁺ 356.08 (m/z). HPLC: 99.8% AUC.

### Synthesis of SR4384

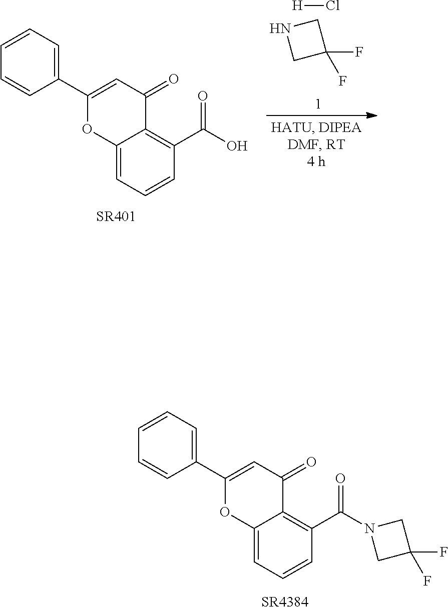

### Preparation of 5-(3,3-difluoroazetidine-1-carbonyl)-2-phenyl-4H-chromen-4-one (SR4384)

To a solution of 4-oxo-2-phenyl-4H-chromene-5-carboxylic acid (SR401) (200 mg, 0.752 mmol) and 3,3-difluoroazetidine hydrochloride (1) (146 mg, 1.12 mmol) in DMF (2 mL) was added DIPEA (0.382 mL, 2.25 mmol) and HATU (427 mg, 1.12 mmol) at 0° C. The reaction mixture was allowed to stir while warming gradually to room temperature for 4 h. The crude residue was purified by reverse phase column chromatography (5%-100% ACN-H₂O), yielding 5-(3,3-difluoroazetidine-1-carbonyl)-2-phenyl-4H-chromen-4-one (SR4384) (110 mg, 40% yield) as an off-white solid. ¹HNMR (600 MHz, CDCl₃) δ 7.95-7.94 (d, 2H), 7.78-7.75 (t, 1H), 7.69-7.67 (d, 1H), 7.59-7.55 (m, 3H), 7.35-7.32 (d, 1H), 6.86 (s, 1H), 4.82-4.80 (m, 1H), 4.46-4.44 (m, 1H), 4.18-4.16 (m, 1H), 4.03-4.02 (m, 1H); MS (M+H)⁺ 341.97 (m/z). HPLC: 99.1% AUC.

### Synthesis of SR4722 (Peak-1 and Peak-2) (Also Referred to Herein as Isomer 1 and Isomer 2)

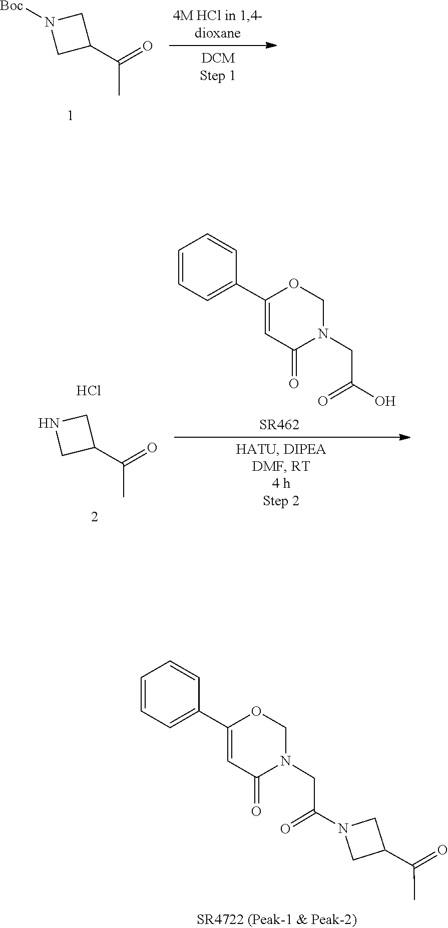

### Step 1: Preparation of 1-(azetidin-3-yl)ethanone hydrochloride (2)

To a stirring solution of tert-butyl 3-acetylazetidine-1-carboxylate (1) (500 mg, 2.50 mmol) in DCM (10 mL) was added 4 M HCl in 1,4-dioxane (2 mL, 8 mmol) at 0° C., then the solution was stirred at room temperature for 4 h. The reaction mixture was concentrated under reduced pressure, yielding 1-(azetidin-3-yl)ethanone hydrochloride (2) (500 mg, crude) as a brown residue, which was taken forward to the next step without further purification.

### Step 2: Preparation of 3-(2-(3-acetylazetidin-1-yl)-2-oxoethyl)-6-phenyl-2H-1,3-oxazin-4(3H)-one (SR4722 Peak-1 and peak-2) (Also Referred to Herein as Isomer 1 and Isomer 2)

To a solution of 2-(4-oxo-6-phenyl-2H-1,3-oxazin-3(4H)-yl)acetic acid (SR462) (200 mg, 0.858 mmol) and 1-(azetidin-3-yl)ethanone hydrochloride (2) (405 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The crude residue was purified by reverse phase column chromatography (5%-100% ACN-H₂O), yielding 3-(2-(3-acetylazetidin-1-yl)-2-oxoethyl)-6-phenyl-2H-1,3-oxazin-4(3H)-one (SR4722-Peak-1 or SR4722, Isomer 1) (30 mg, 11% yield) as a white solid and 3-(2-(3-acetylazetidin-1-yl)-2-oxoethyl)-6-phenyl-2H-1,3-oxazin-4(3H)-one (SR4722-Peak-2 or SR4722, Isomer 2) (10 mg, 3% yield) as a white solid.

### SR4722 (Isomer 1 or Peak 1):

¹HNMR (600 MHz, CDCl₃) δ 7.69-7.68 (d, 2H), 7.48-7.40 (m, 3H), 5.93 (s, 1H), 5.47-5.46 (d, 1H), 5.38-5.37 (d, 1H), 4.45-4.39 (m, 2H), 4.30-4.27 (d, 1H), 4.23-4.20 (t, 1H), 4.10-4.08 (t, 1H), 3.89-3.86 (d, 1H), 3.55-3.53 (m, 1H), 2.22 (s, 3H). MS (M+H)⁺ 315.10 (m/z). HPLC: 97.8% AUC.

### SR4722 (Isomer 2 or Peak 2):

¹HNMR (600 MHz, CDCl₃) δ 7.70-7.69 (m, 2H), 7.50-7.42 (m, 3H), 6.59 (s, 1H), 6.12 (s, 1H), 6.02 (s, 1H), 5.93 (s, 1H), 5.30 (s, 1H), 4.10 (s, 2H), 4.07-4.06 (d, 2H), 2.34 (s, 3H). MS (M+H)⁺ 315.10 (m/z). HPLC: 99.4% AUC.

### Synthesis of SR7255

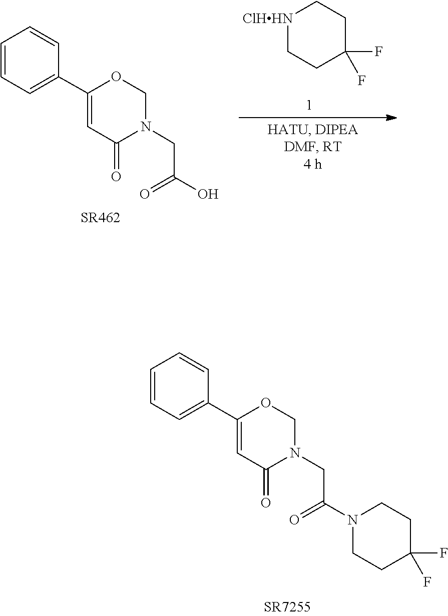

### Preparation of 3-(2-(4,4-difluoropiperidin-1-yl)-2-oxoethyl)-6-phenyl-2H-1,3-oxazin-4(3H)-one

To a solution of 2-(4-oxo-6-phenyl-2H-1,3-oxazin-3(4H)-yl)acetic acid (SR462) (200 mg, 0.858 mmol) and 4,4-difluoropiperidine hydrochloride (1) (473 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The crude residue was purified by reverse phase column chromatography (5%-100% ACN-H₂O), yielding 3-(2-(4,4-difluoropiperidin-1-yl)-2-oxoethyl)-6-phenyl-2H-1,3-oxazin-4(3H)-one (SR7255) (110 mg, 38% yield) as a white solid. ¹HNMR (600 MHz, CDCl₃) δ 7.72-7.71 (d, 2H), 7.49-7.42 (m, 3H), 5.97 (s, 1H), 5.44 (s, 2H), 4.36 (s, 2H), 3.75 (s, 2H), 3.64 (s, 2H), 2.09-1.99 (m, 4H). MS (M+H)⁺ 337.03 (m/z). HPLC: 97.4% AUC.

### Synthesis of SR5955

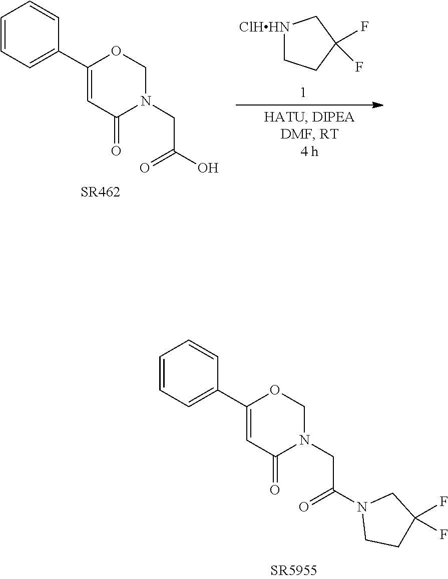

### Preparation of 3-(2-(3,3-difluoropyrrolidin-1-yl)-2-oxoethyl)-6-phenyl-2H-1,3-oxazin-4(3H)-one (SR5955)

To a solution of 2-(4-oxo-6-phenyl-2H-1,3-oxazin-3(4H)-yl)acetic acid (SR462) (200 mg, 0.858 mmol) and 3,3-difluoropyrrolidine hydrochloride (1) (437 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The crude residue was purified by reverse phase column chromatography (5%-100% ACN-H₂O), yielding 3-(2-(3,3-difluoropyrrolidin-1-yl)-2-oxoethyl)-6-phenyl-2H-1,3-oxazin-4(3H)-one (SR5955) (130 mg, 45% yield) as a white solid. ¹HNMR (600 MHz, CDCl₃) δ 7.70-7.69 (d, 2H), 7.49-7.41 (m, 3H), 5.95 (s, 1H), 5.45 (s, 2H), 4.29 (s, 1H), 4.21 (s, 1H), 3.93-3.71 (m, 4H), 2.51-2.32 (m, 2H). MS (M+H)⁺ 323.05 (m/z). HPLC: 98.7% AUC.

### Synthesis of SR7280

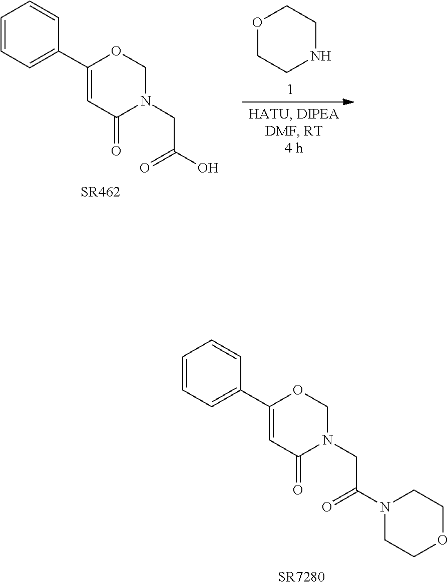 [SMILES: O=C(O)CN1COC(c2ccccc2)=CC1=O ; C1COCCN1 ; O=C1C=C(c2ccccc2)OCN1CC(=O)N1CCOCC1]

### Preparation of 3-(2-morpholino-2-oxoethyl)-6-phenyl-2H-1,3-oxazin-4(3H)-one (SR7280)

To a solution of 2-(4-oxo-6-phenyl-2H-1,3-oxazin-3(4H)-yl)acetic acid (SR462) (200 mg, 0.858 mmol) and morpholine (1) (261 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The crude residue was purified by reverse phase column chromatography (5%-100% ACN-H₂O), yielding 3-(2-morpholino-2-oxoethyl)-6-phenyl-2H-1,3-oxazin-4(3H)-one (SR7280) (100 mg, 38% yield) as a white solid. ¹HNMR (600 MHz, CDCl₃) δ 7.71-7.69 (d, 2H), 7.49-7.41 (m, 3H), 5.96 (s, 1H), 5.43 (s, 2H), 4.35 (s, 2H), 3.73-3.51 (m, 8H). MS (M+H)⁺ 303.11 (m/z). HPLC: 98.0% AUC.

### Synthesis of SR4511

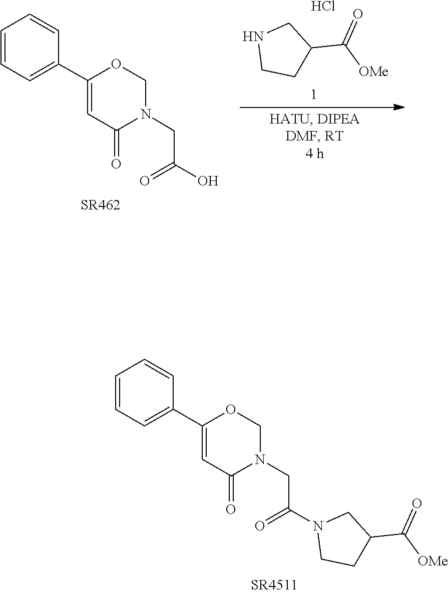 [SMILES: Cl ; O=C(O)CN1COC(c2ccccc2)=CC1=O ; COC(=O)C1CCNC1 ; COC(=O)C1CCN(C(=O)CN2COC(c3ccccc3)=CC2=O)C1]

### Preparation of methyl 1-(2-(4-oxo-6-phenyl-2H-1,3-oxazin-3(4H)-yl)acetyl)pyrrolidine-3-carboxylate (SR4511)

To a solution of 2-(4-oxo-6-phenyl-2H-1,3-oxazin-3(4H)-yl)acetic acid (SR462) (200 mg, 0.858 mmol) and methyl pyrrolidine-3-carboxylate hydrochloride (1) (424 mg, 2.57 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The crude residue was purified by reverse phase column chromatography (5%-100% ACN-H₂O), yielding methyl 1-(2-(4-oxo-6-phenyl-2H-1,3-oxazin-3(4H)-yl)acetyl)pyrrolidine-3-carboxylate (SR4511) (100 mg, 34% yield) as a white solid. ¹HNMR (600 MHz, CDCl₃) δ 7.70-7.69 (d, 2H), 7.49-7.42 (m, 3H), 5.95 (s, 1H), 5.50-5.41 (dd, 2H), 4.45-4.40 (t, 1H), 4.15-4.11 (dd, 1H), 3.79-3.50 (m, 7H), 3.20-3.06 (m, 1H), 2.30-2.15 (m, 2H). MS (M+H)⁺ 344.98 (m/z). HPLC: 95.7% AUC.

### Synthesis of SR7301

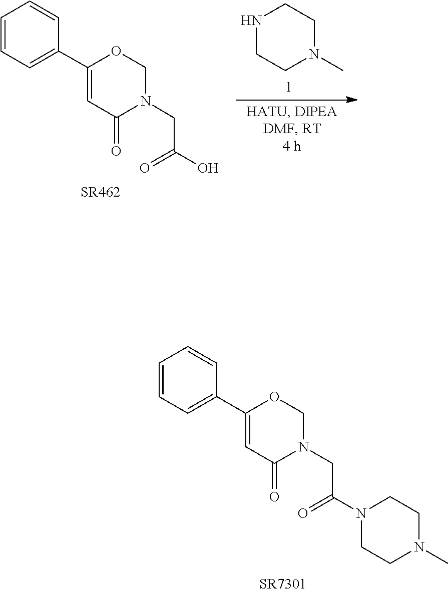

### Preparation of 3-(2-(4-methylpiperazin-1-yl)-2-oxoethyl)-6-phenyl-2H-1,3-oxazin-4(3H)-one (SR7301)

To a solution of 2-(4-oxo-6-phenyl-2H-1,3-oxazin-3(4H)-yl)acetic acid (SR462) (200 mg, 0.858 mmol) and 1-methylpiperazine (1) (125 mg, 1.28 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h.

The crude residue was purified by reverse phase column chromatography (5%-100% ACN-H₂O), yielding 3-(2-(4-methylpiperazin-1-yl)-2-oxoethyl)-6-phenyl-2H-1,3-oxazin-4(3H)-one (SR7301): (110 mg, 40% yield) as a white solid. ¹HNMR (600 MHz, DMSO-d₆) δ 7.80-7.78 (m, 2H), 7.54-7.47 (m, 3H), 6.07 (s, 1H), 5.38 (s, 2H), 4.35 (s, 2H), 3.44-3.32 (m, 4H), 2.36-2.33 (t, 2H), 2.29-2.26 (t, 2H), 2.20 (s, 3H). MS (M+H)⁺ 316.06 (m/z). HPLC: 99.8% AUC.

### Synthesis of SR7254

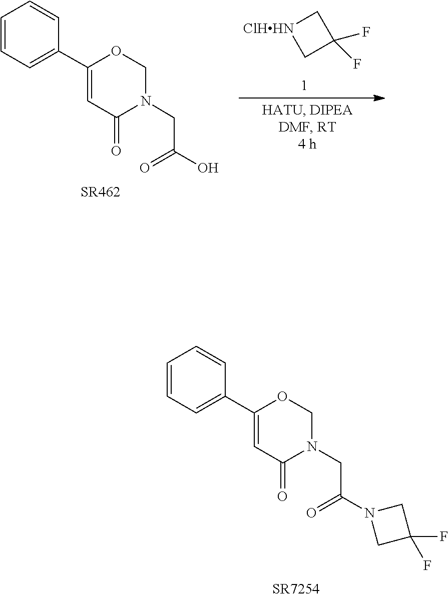

### Preparation of 3-(2-(3,3-difluoroazetidin-1-yl)-2-oxoethyl)-6-phenyl-2H-1,3-oxazin-4(3H)-one (SR7254)

To a solution of 2-(4-oxo-6-phenyl-2H-1,3-oxazin-3(4H)-yl)acetic acid (SR462) (200 mg, 0.858 mmol) and 3,3-difluoroazetidine hydrochloride (1) (128 mg, 1.25 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The crude residue was purified by reverse phase column chromatography (5%-100% ACN-H₂O), yielding 3-(2-(3,3-difluoroazetidin-1-yl)-2-oxoethyl)-6-phenyl-2H-1,3-oxazin-4(3H)-one (SR7254): (100 mg, 38% yield) as a white solid. ¹HNMR (600 MHz, CDCl₃) δ 7.71-7.70 (d, 2H), 7.50-7.43 (m, 3H), 5.96 (s, 1H), 5.45 (s, 2H), 4.67-4.63 (t, 2H), 4.40-4.35 (t, 2H), 4.14 (s, 2H). MS (M+H)⁺ 309.20 (m/z). HPLC: 98.0% AUC.

### Synthesis of SR7302

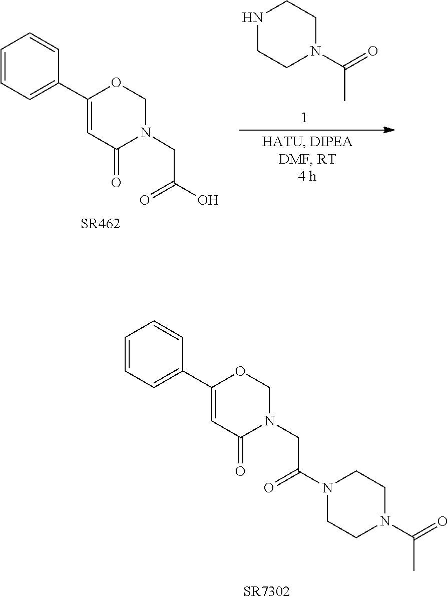

### Preparation of 3-(2-(4-acetylpiperazin-1-yl)-2-oxoethyl)-6-phenyl-2H-1,3-oxazin-4(3H)-one (SR7302)

To a solution of 2-(4-oxo-6-phenyl-2H-1,3-oxazin-3(4H)-yl)acetic acid (SR462) (200 mg, 0.858 mmol) and 1-(piperazin-1-yl)ethanone (1) (160 mg, 1.25 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The crude residue was purified by reverse phase column chromatography (5%-100% ACN-H₂O), yielding 3-(2-(4-acetylpiperazin-1-yl)-2-oxoethyl)-6-phenyl-2H-1,3-oxazin-4(3H)-one (SR7302): (100 mg, 38% yield) as a white solid. ¹HNMR (600 MHz, CDCl₃) δ 7.71-7.70 (d, 2H), 7.50-7.42 (m, 3H), 5.97 (s, 1H), 5.44 (s, 2H), 4.38-4.37 (m, 2H), 3.71-3.50 (m, 8H), 2.13 (s, 3H). MS (M+H)⁺ 344.15 (m/z). HPLC: 99.8% AUC.

### Synthesis of SR7232

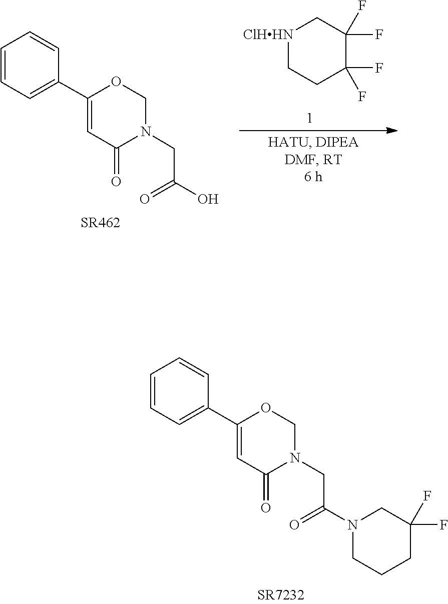

### Preparation of 3-(2-(3,3-difluoropiperidin-1-yl)-2-oxoethyl)-6-phenyl-2H-1,3-oxazin-4(3H)-one (SR7232)

To a solution of 2-(4-oxo-6-phenyl-2H-1,3-oxazin-3(4H)-yl)acetic acid (SR462) (200 mg, 0.858 mmol) and 3,3-difluoropiperidine hydrochloride (1) (473 mg, 3.00 mmol) in DMF (2 mL) was added DIPEA (0.44 mL, 2.57 mmol) and HATU (484 mg, 1.27 mmol) at 0° C. The reaction mixture was allowed to stir while warming gradually to room temperature for 4 h. The crude residue was purified by reverse phase column chromatography (5%-100% ACN-H₂O), yielding 3-(2-(3,3-difluoropiperidin-1-yl)-2-oxoethyl)-6-phenyl-2H-1,3-oxazin-4(3H)-one (SR7232): (110 mg, 39% yield) as a white solid. ¹HNMR (600 MHz, DMSO-d₆) δ 7.79-7.78 (d, 2H), 7.54-7.47 (m, 3H), 6.06 (s, 1H), 5.38 (s, 2H), 4.40-4.28 (d, 2H), 3.81-3.77 (m, 2H), 3.48 (s, 2H), 2.08-2.05 (m, 2H), 1.75-1.71 (m, 1H), 1.64-1.60 (m, 1H). MS (M+H)⁺ 337.11 (m/z). HPLC: 99.8% AUC.

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicant reserves the right to challenge the accuracy and pertinence of any cited reference.

### REFERENCES

Almeida M F, Piehler T, Carstens K E, Zhao M, Samadi M, Dudek S M, et al. Distinct and dementia-related synaptopathy in the hippocampus after military blast exposures. Brain pathology (Zurich, Switzerland). 2021; 31:e12936.
Hwang J, Estick C M, Ikonne U S, Butler D, Pait M C, Elliott L H, et al. The Role of Lysosomes in a Broad Disease-Modifying Approach Evaluated across Transgenic Mouse Models of Alzheimer's Disease and Parkinson's Disease and Models of Mild Cognitive Impairment. Int J Mol Sci. 2019; 20:4432.
Farizatto K L G, Ikonne U S, Almeida M F, Ferrari M F R, Bahr B A. Aβ42-mediated proteasome inhibition and associated tau pathology in hippocampus are governed by a lysosomal response involving cathepsin B: Evidence for protective crosstalk between protein clearance pathways. PLoS One. 2017; 12:e0182895.
Mueller-Steiner S, Zhou Y, Arai H, Roberson E D, Sun B, Chen J, et al. Antiamyloidogenic and Neuroprotective Functions of Cathepsin B: Implications for Alzheimer's Disease. Neuron. 2006; 51:703-14.
Rodriguez-Navarro J A, Rodriguez L, Casarejos M J, Solano R M, Gómez A, Perucho J, et al. Trehalose ameliorates dopaminergic and tau pathology in parkin deleted/tau overexpressing mice through autophagy activation. Neurobiol Dis. 2010; 39:423-38.
Butler D, Hwang J, Estick C, Nishiyama A, Kumar S S, Baveghems C, et al. Protective effects of positive lysosomal modulation in Alzheimer's disease transgenic mouse models. PLoS One. 2011; 6:e20501.
Kruger U, Wang Y, Kumar S, Mandelkow E-M. Autophagic degradation of tau in primary neurons and its enhancement by trehalose. Neurobiol Aging. 2012; 33:2291-305.
Embury C M, Dyavarshetty B, Lu Y, Wiederin J L, Ciborowski P, Gendelman H E, et al. Cathepsin B Improves β-Amyloidosis and Learning and Memory in Models of Alzheimer's Disease. Journal of Neuroimmune Pharmacology. 2017; 12:340-52.
Johnson E A, Nowar R, Viola K L, Huang W, Zhou S, Bicca M A, et al. Inhibition of amyloid beta oligomer accumulation by NU-9: A unifying mechanism for the treatment of neurodegenerative diseases. Proceedings of the National Academy of Sciences. 2025; 122:e2402117122.
Bendiske J, Bahr B A. Lysosomal activation is a compensatory response against protein accumulation and associated synaptopathogenesis—an approach for slowing Alzheimer disease? J Neuropathol Exp Neurol. 2003; 62:451-63.
Butler D, Bendiske J, Michaelis M L, Karanian D A, Bahr B A. Microtubule-stabilizing agent prevents protein accumulation-induced loss of synaptic markers. Eur J Pharmacol. 2007; 562:20-7.
Ryzhikov S, Bahr B A. Gephyrin alterations due to protein accumulation stress are reduced by the lysosomal modulator Z-Phe-Ala-diazomethylketone. J Mol Neurosci. 2008; 34:131-9.
Wang J, Zhang Y, Tang L, Zhang N, Fan D. Protective effects of resveratrol through the up-regulation of SIRT1 expression in the mutant hSOD1-G93A-bearing motor neuron-like cell culture model of amyotrophic lateral sclerosis. Neurosci Lett. 2011; 503:250-5.
Wu Y, Li X, Zhu J X, Xie W, Le W, Fan Z, et al. Resveratrol-activated AMPK/SIRT1/autophagy in cellular models of Parkinson's disease. Neurosignals. 2011; 19:163-74.
Porquet D, Casadesns G, Bayod S, Vicente A, Canudas A M, Vilaplana J, et al. Dietary resveratrol prevents Alzheimer's markers and increases life span in SAMP8. Age (Dordr). 2013; 35:1851-65.
Mancuso R, del Valle J, Modol L, Martinez A, Granado-Serrano A B, Ramirez-Nunez O, et al. Resveratrol improves motoneuron function and extends survival in SOD1(G93A) ALS mice. Neurotherapeutics. 2014; 11:419-32.
Vingtdeux V, Giliberto L, Zhao H, Chandakkar P, Wu Q, Simon J E, et al. AMP-activated protein kinase signaling activation by resveratrol modulates amyloid-beta peptide metabolism. J Biol Chem. 2010; 285:9100-13.
Deng H, Mi M T. Resveratrol Attenuates A025-35 Caused Neurotoxicity by Inducing Autophagy Through the TyrRS-PARP1-SIRT1 Signaling Pathway. Neurochem Res. 2016; 41:2367-79.
Verwaerde, P. et al., ChemMedChem. 2025 Mar. 27:e202400891.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A compound having a structure of Formula (I) or Formula (II): 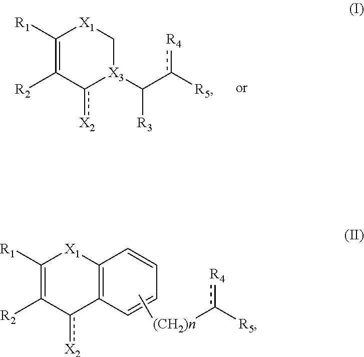
wherein:
n is 0, 1, 2, 3, 4, 5, or 6;
each [symbol] is independently a double or single bond;
X₁ is O or methylene;
X₂ is H, OH, SH, NH₂, O, S, or NH, subject to the proviso that when the [symbol] to which X₂ is attached is a single bond, X₂ is selected from H, OH, SH, and NH₂ and when the [symbol] to which X₂ is attached is a double bond, X₂ is O, S or NH;
X₃ is CH or N;
R₁, R₂, and R₃ are independently selected from the group comprising H, OH, alkyl, substituted alkyl, alkoxy, aralkyl, substituted aralkyl, aryl, and substituted aryl;
R₄ is OH or O, subject to the proviso that R₄ is OH when the [symbol] to which R₄ is attached is a single bond and O when the [symbol] to which R₄ is attached is a double bond; and
R₅ is selected from H, OH, alkyl, substituted alkyl, aralkyl, substituted aralkyl, aryl, substituted aryl, and N(R₆)₂, wherein each R₆ is independently selected from H, acyl, alkyl, substituted alkyl, aralkyl, substituted aralkyl, aryl, and substituted aryl, or wherein the two R₆ together form a substituted or unsubstituted alkylene or aralkylene group; or
a pharmaceutically acceptable salt thereof.

2. The compound of claim 1, wherein the compound of Formula (I) has a structure of Formula (Ia): 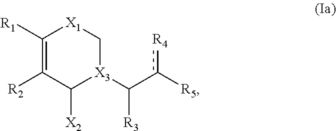
wherein: [symbol]
is a double or single bond;
X₁ is O or methylene, optionally wherein X₁ is O;
X₂ is H or OH;
X₃ is CH or N;
R₁, R₂, and R₃ are independently selected from the group comprising H, alkyl, substituted alkyl, aralkyl, substituted aralkyl, aryl, and substituted aryl;
R₄ is OH or O, subject to the proviso that R₄ is OH when the [symbol] to which R₄ is attached is a single bond and O when the [symbol] to which R₄ is attached is a double bond; and
R₅ is selected from H, OH, alkyl, substituted alkyl, aralkyl, substituted aralkyl, aryl, substituted aryl, and N(R₆)₂, wherein each R₆ is independently selected from H, acyl, alkyl, substituted alkyl, aralkyl, substituted aralkyl, aryl, and substituted aryl, or wherein the two R₆ together form a substituted or unsubstituted alkylene or aralkylene group; or
a pharmaceutically acceptable salt thereof.

3. The compound of claim 1, wherein the compound of Formula (I) has a structure of Formula (Ib): 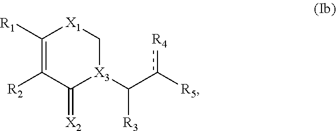
wherein [symbol] is a double or single bond; X₁, X₃, R₁, R₂, R₃, R₄, and R₅ are as defined for Formula (I) and where X₂ is selected from O, S, and NH, optionally wherein X₂ is O, further optionally wherein X₁ is O and/or X₃ is N.

4. The compound of claim 3, wherein X₁ and X₂ are each O and X₃ is N and the compound of Formula (Ib) has a structure of Formula (III): 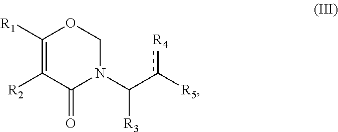
wherein [symbol] is a double or single bond and R₁, R₂, R₃, R₄, and R₅ are as defined in claim 1.

5. The compound of claim 4, wherein the compound of Formula (III) has a structure of Formula (IIIa): 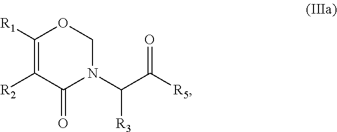
wherein R₁, R₂, R₃, and R₅ are as defined in claim 1.

6. The compound of claim 5, wherein R₅ is selected from OH, lower alkyl, and N(R₆)₂, wherein the two R₆ together form a substituted or unsubstituted 4-, 5-, 6-, 7-, or 8-membered ring.

7. The compound of claim 6, wherein R₅ is 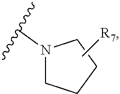
wherein R₇ is selected from H and C(═O)R₈, wherein R₈ is selected from alkyl and substituted alkyl, optionally wherein R₈ is methyl.

8. The compound of claim 4, wherein the compound of Formula (III) has a structure of Formula (IIIb): 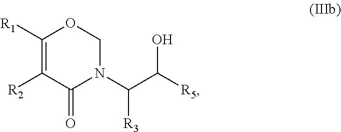
wherein R₁, R₂, R₃, and R₅ are as defined in claim 1.

9. The compound of claim 1, wherein R₃ is H, benzyl, or substituted benzyl.

10. The compound of claim 9, wherein R₅ is amino-substituted alkyl, optionally —CH₂—N(R₉)(R₁₀), wherein R₉ and R₁₀ are independently selected from H, alkyl, substituted alkyl, aralkyl, substituted aralkyl, aryl, substituted aryl, and —C(═O)R₁₁, wherein R₁₁ is selected from alkyl, substituted alkyl, aralkyl, substituted aralkyl, aryl, and substituted aryl.

11. The compound of claim 10, wherein R₅ is —CH₂N(R₉)(R₁₀), wherein R₉ is substituted or unsubstituted aralkyl, optionally —CH₂—R₁₂, wherein R₁₂ is substituted or unsubstituted phenyl, naphthyl, or pyridine; and R₁₀ is —C(═O)R₁₁, wherein R₁₁ is alkyl or substituted alkyl.

12. The compound of claim 1, wherein the compound of Formula (II) has a structure of Formula (IIa): 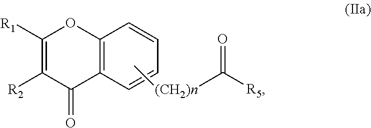
wherein n, R₁, R₂, and R₅ are as defined in claim 1.

13. The compound of claim 12, wherein n is 1 and/or wherein R₅ is 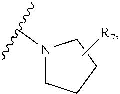
wherein R₇ is selected from H and —C(═O)R₈, wherein R₈ is selected from alkyl and substituted alkyl, optionally wherein R₈ is methyl.

14. The compound of claim 1, wherein one of R₁ and R₂ is H and one of R₁ and R₂ is aralkyl, substituted aralkyl, aryl, or substituted aryl, optionally wherein one of R₁ and R₂ is phenyl or naphthyl.

15. The compound of claim 1, wherein the compound is selected from the group consisting of SR466, SR71, SR70, SR72, SR401, SR459, SR466, SR462, SR103, SR103B, SR104, SR105, SR107, SR111, SR720, SR720-1, SRB3, SR70, SR71, SR72, SR4721, SR4722, SR7254, SR7280, SR7255, SR5955, SR5981, SR-J1, SR438, SR498, SR490, SR495, SR496, SR206, SR4386, SR4385, SR4384, SR4511, SR7301, SR7302, SR7232, and SR710, optionally SR459, SR466, SR462, and SR438.

16. The compound of claim 15, wherein the compound is SR438, SR498, SR459 or SR466, optionally wherein the compound is SR438-S, SR438-R, SR498-S, SR498-R, SR459-S, SR459-R, SR466-S, or SR466-R.

17. The compound of claim 15, wherein the compound is SR4722, isomer 1 and/or SR4722, isomer 2.

18. A pharmaceutical composition comprising a compound of claim 1 and a pharmaceutically acceptable carrier.

19. A method of positively modulating cathepsin β in a sample comprising cathepsin β, the method comprising contacting the sample with a compound of claim 1 or a pharmaceutical composition thereof.

20. The method of claim 19, wherein the contacting provides increased degradation of Aβ42, increased clearance of tau, and/or increased clearance of α-synuclein.

21. A method of treating or preventing a neurodegenerative disease, disorder or condition in a subject in need of treatment or at risk thereof, wherein the method comprises administering to the subject an effective amount of a compound of claim 1 or a pharmaceutical composition thereof; optionally wherein the neurodegenerative disease, disorder, or condition is selected from Alzheimer's disease, Parkinson's disease, Huntington's disease, amyotrophic lateral sclerosis, mild cognitive impairment (MCI) or traumatic brain injury (TBI), optionally wherein the compound is SR459 or SR466.
